(19) United States
(12) Patent Application Publication
(10) Pub. No.: US 2011/0052678 A1
(43) Pub. Date: Mar. 3, 2011

(54) METHOD FOR TREATING AGE RELATED MACULAR DEGENERATION

(76) Inventors: Totada R. Shantha, McDonough, GA (US); Jessica Shantha, McDonough, GA (US); Erica Maya Shantha, McDonough, GA (US)

(21) Appl. No.: 12/940,247

(22) Filed: Nov. 5, 2010

(51) Int. Cl. A61K 38/28; A61K 33/00; A61K 38/30; A61K 39/395; A61K 33/30; A61K 33/04; A61K 9/127; A61K 38/44; A61K 33/06

(52) U.S. Cl. 424/450; 514/5.9; 514/6.5; 424/600; 514/8.6; 424/133.1; 424/145.1; 424/141.1; 424/642; 424/702; 424/94.4; 424/682

(57) ABSTRACT

A method for treating age related macular degeneration uses insulin preparation applied topically in a therapeutically effective amount to an affected conjunctival sac of the eye. The topically effective dose is delivered to the fovea centralis and macula lutea. In other embodiments, additional therapeutic, pharmaceutical, biochemical, nutriceutical, biological (monoclonal antibodies and others) agent or compound, and organic and inorganic agents are also applied to the afflicted site through the conjunctival sac and choroidal vascular system of the eye. IGF-1 may be applied as well with or without insulin to treat ARMD.

### FIELD OF THE INVENTION

This invention relates to treatment of age related macular degenerative (ARMD) diseases of the eye affecting the retinal function in humans and animals.

### BACKGROUND OF THE INVENTION

Age related macular degeneration (ARMD) is a retinal eye disease that involves the macula involved in central vision. This is the most common cause of blindness. The macula is a small spot in the central area of the retina located at the back of the eye. The macula is responsible for sight in the centre of the field of vision. Symptoms of ARMD depend upon the phase of the ARMD. The most common symptom comprises straight lines in the field of vision yet appears wavy. The type in books, magazines and newspapers appears blurry. The dark or empty spaces block the centre of vision.

People with macular degeneration may find difficulty in doing simple everyday activities requiring sharp vision. In the United States, macular degeneration affects over 13 million people. ARMD is the leading cause of visual impairment for persons age 75 and older (30% affected). Above the age of 65, individuals lose at least 10% of their central vision which results in the visual impairment which results with the development of macular degeneration. Macular degeneration affects one in 10 people over the age of 65, as the average age of the U.S. population continues to increase so does the number of people suffering from ARMD. More than 200,000 new cases develop annually. ARMD is more common in non-Hispanic whites than in blacks or Mexican-Americans.

According to the forecast, Age-Related Macular Degeneration cases will increase from 13 million in 2010 to 17.8 million by 2050. In non-vitamin-receiving individuals, cases of choroidal neovascularization (CNV) with geographic atrophy will be increased from 1.7 million in 2010 to 3.8 million in 2050. It is estimated that the cases of visual impairment and blindness will increase from 620,000 in 2010 to 1.6 million in 2050 when given no treatment (David B. Rein, et al; for the Vision Health Cost-Effectiveness Study Group The Potential Impact of New Treatments Arch Ophthalmol. 2009; 127(4):533-540).

What causes ARMD isn't known. A tendency to develop macular degeneration may be seen in some families due to genetic factors. There are factors which can increase the risk of developing ARMD such as: genetics—a family history of macular degeneration, being, female, possess a light skin tone, widespread exposure to UV light, high blood pressure, Aging—an estimated 10% of ARMD are under the age of 50, Diabetes, elevated total serum cholesterol, higher body mass index (BMI), and Smoking. The smoking has consistently been associated with higher ARMD risk compared to other risk factors.

Wanda Hamilton, the Executive Director of ARMD Alliance International, clarifies that smoking and genetics play the greatest roles in determining if you may be at risk of developing ARMD. “If you have a particular gene make-up and you smoke, you could be up to 144 times more likely to get ARMD. If you have other genes and you smoke, you could be up to seven times more likely than non-smokers to get the disease.”

Cataract removal may create a higher risk for ARMD with the removal of the lens allows previously filtered light to pass unobstructed to the retina. At times Transition lenses, also, called photochromic lenses are prescribed for ARMD. These lenses change from nearly clear indoors to darker outdoors. This type of lens cuts the glare which provides clarity of vision and comfort for someone with macular degeneration. Ophthalmologists perform dilated eye exams, ophthalmoscopic exam, fluorescein angiograms, and use Amsler grids as well as other tests to diagnose ARMD.

There are measures that one can take to reduce the risk of ARMD. The following health measures may prevent, delay or curtail the onset and the effects of ARMD. They are: Don't smoke, Always wear sunglasses (use both blue and UV light blocking glasses) even on cloudy days and in the winter, wear hats and decrease your exposure to the Sun. The individual needs to keep the blood pressure and cholesterol at the proper level, to keep weight at a healthy level by Exercise for 30 minutes at least four times weekly to help maintain ideal body weight and optimal blood pressure. The reduction dietary fat to 20-25% of total dietary calories, decrease red meats, whole milk, cheese, and butter while increasing consumption of omega-3 fatty acids (e.g., cold-water fish, canola oil, etc.) reduce the incidence or delay the development of ARMD, The individual needs to consume abundance of fruits and vegetables, especially green, leafy ones. Reduction consuming of junk food (processed foods) and eat two or more servings of fish which are high in omega 3 every week like salmon and mackerel. Living a healthy lifestyle and lifelong UV protection are essential to reducing ones risk of developing ARMD.

Simple natural dietary habits can reduce the risk of developing ARMD. Vitamins A, C and E all offer benefits for overall eye health. Take vitamin C (500 mg), vitamin E (400 IU), beta-carotene (15 mg) or vitamin A, and zinc (80 mg as zinc oxide), daily. Vitamin A can help to reduce the risks of cataracts and night blindness. The deficiency of Vitamin A has been implicated in blindness and corneal ulcers. Vitamin C reduces pressure in glaucoma, slows age-macular related degeneration (ARMD) and prevents cataracts. Vitamin C is a strong antioxidant that is highly concentrated in the lens of the eye. Vitamin C is used by the muscles of the eye. Vitamin E helps to reduce the risk of macular degeneration and cataracts. These supplements have not been shown to prevent ARMD; however, these supplements slow the progression of the established disease. Two important antioxidants for eye health that must be acquired in the diet are lutein and zeaxanthin. They are found in leafy, green vegetables such as spinach, kale and fresh parsley, yellow fruits and vegetables. Minerals are needed to help the body metabolize vitamins, balance nutrition, and hormones. Critical minerals for eye health include zinc and selenium.

Other important supplements for eye health are lutein, bioflavonoids and carotenoid. Natural supplements for eye health should include bilberry and blueberry, which contains antioxidant compounds that help maintain the strength and the structure of eye capillaries and retina. The grape seed extract is a natural powerful antioxidant. Proanthocyanidins are recommended for their powerful vascular strengthening abilities and antioxidant activity. Blood sugar should be kept normal. The patient should avoid MSG, hydrogenated oils, artificial food flavoring and coloring agents. Smokers should avoid taking beta-carotene (Age Related Eye Disease Study Research Group. A randomized, placebo-controlled, clinical trial of high-dose supplementation with vitamins C and E, beta carotene and zinc for age related macular degeneration and vision loss Arch Ophthalmol 2001; 119:1417-36). The patient needs to eat more green leafy vegetables and supplement with use of lutein-zeaxanthin supplements. These pigments help to reduce the effects of blue light as it penetrates the macula and RPE.

ARMD affects the macula lutea (FIG. 1). The center of the macula is called the fovea centralis which is the area of location for the cones photoreceptors. There are no rods located in the fovea centralis. The fovea is the place of sharpest and most sensitive visual acuity. Macula is a highly specialized retina located at the back of the eye directly facing the center of the cornea and lens. It is responsible for sight in the centre of the field of vision. Macula is approximately an eighth of an inch in diameter. The macula has densely packed photoreceptors cone photoreceptors that collect light which are responsible for central vision. The peripheral retina is composed mainly of rods which are the light-sensitive cells responsible for side and night vision. The macula is one hundred times more sensitive to detail than the peripheral retina. The human macula has 7 million special cones in each eye and a dense concentration of ganglion cells which permit high resolution of visual acuity compared to 110-120 million rods in the rest of the retina in each eye.

In a healthy macula, the clear layer of the retina on the inside of the eye is nourished and maintained by the retinal pigment epithelium (RPE). Behind the pigment epithelium is the non cellular Bruch's membranous layer and highly vascular choroid which contains the rich net work of blood vessels and choroidal lamellar cells (between the choirdal BV and Sclera). These are the extension of the pia-arachnoid membrane of the optic nerve (Shantha T R and Bourne G H: Histological and Histochemical studies of the choroid of the eye and its relations to the pia-arachnoid mater of the central nervous system and Perineural epithelium of the peripheral nervous system. Acta Anat 61:379-398 (1965). Shantha T R and Bourne G H: Arachnoid villi in the optic nerve of man and monkey. Expt Eye Res 3:31-35 (1964)) that transport nourishment to and carry out metabolic waste away from the retina (FIG. 1).

Three forms of macular degeneration have been identified: 1. atrophic, non-exudative-dry form occurs in 85 to 90% of patients with macular degeneration. 2. Exudative commonly known as wet form occurs in 10% of patients usually treated with laser surgery; and 3. Pigment epithelial detachment associated (PED) ARMD which occurs in less than 5% of the patients resulting in retinal detachment. In the dry form, there is a breakdown or thinning of the retinal pigment epithelial cells (RPE) in the macula, hence the term “atrophy”. These RPE cells are important for the proper functioning of the retina. They metabolically support the overlying photoreceptor. In the wet form of macular-degeneration, abnormal blood vessels grow uncontrolled called subretinal neo-vascularization (SRNV) under the retina. They lift the retina up with loss of ability to see (FIG. 2).

In the normal choroid, the large blood vessels (BV) have intact thick vessel walls. The choriocapillaries coming out of the main choroidal BV have fenestrations or openings in their walls allowing easily the contents of the circulating blood to leak out to the extracellular Bruch's membranous space on the surface of RPE which in turn supplies nutrient to the underlying retinal photoreceptors cells (FIG. 1). In patients with ARMD, new blood vessels proliferate from these choriocapillaries through Bruch's membrane adjacent to the retinal pigment epithelium (RPE), and form a mass of vascular plexus (FIG. 2). The resulting choroidal neovascularizations (new vessels in the choroid) occur with around 10% of the patients with ARMD. Such neovascularizations is seen in patients with pathologic myopia, ocular histoplasmosis syndrome, and other idiopathic conditions. The fluid from these BV (blood, cellular elements, electrolytes, plasma fluid, drugs in plasma if the person on medications orally or as ophthalmic drops) leaks to the surrounding tissue. This fluid can increase, build up pressure, and press on the RPE and retina, resulting in their detachment leading to defective vision and blindness (FIG. 2).

Ultimately, the fluid may be absorbed and drying which leads to scarring. In the dry type of ARMD, the RPE cells die resulting atrophic ARMD. As ARMD advances, the person loses the sharp, central vision needed to see straight ahead and to engage in such activities as reading, needlework and driving. With no appropriate treatment, many of them become legally blind in both types of ARMD. This condition is the leading cause of loss vision in US above the age sixty years or older.

In “dry” macular degeneration, there is a slow breakdown of photoreceptors cone reducing central vision. About 90 percent of people with macular degeneration have this dry form. Treatment with additional supplemental vitamins and minerals may slow the progress of the disease. As “dry” macular degeneration worsens, new, fragile blood vessels (BV) grow beneath the macula from the choroid above the pigment layer. The dead neurons allow the BV to grow. The cones may be anti angiogenic and their destruction results in continued unabated angiogenesis leading to the pathology. These new blood vessels often leak blood and fluid, which causes further damage to the macula which leads to loss of central vision. This form of the disease is known as “wet” macular degeneration. Although, the “wet” macular degeneration is found in 10% of the ARMD. This accounts for 90 percent of all blindness in ARMD.

Wet ARMD treatment consists of laser surgery or Photodynamic therapy to destroy new blood vessels. Only about 15 percent of patients with the “wet” form of macular degeneration are suitable for laser surgery because the new blood vessels grow too close to the macula where the visual image is focused. Laser treatment can only be applied after sight-threatening changes have occurred. Despite laser treatment, the disease and loss of vision may progress unabated. Once vision is lost, it cannot be restored. No medical treatment is currently available for macular degeneration hence we bring this new method of treatment. We call the ARMD “The diabetes of the eye” Retinal pigment epithelial cells, (RPE) are nearly black due to melanin pigment. They form a layer that recharges the photoreceptor cells of the eye after they are exposed to light. The photoreceptors contain molecules called photopigments in their outer segments in close proximity to the photoreceptors. When light (photons) strikes these molecules, they absorb the light and change shape (uncoiling), sending a signal to the brain indicating they've “seen” light. Once a photopigment molecule absorbs light, it needs to get recharged.

The photopigment molecule is shuttled out of the photoreceptor and down to the RPE cells. The RPE cells recharge the photopigment molecules which send them back to the photoreceptors to start the process again. This process takes 20 minutes. In addition, the RPE layer keeps the photoreceptors healthy by collecting, storing and disposing toxic waste products that are produced during the process of regenerating the photopigment. In macular degeneration for reasons that are not yet completely clear, the RPE cells are unable to provide this support for the photoreceptors and both of these cells eventually die. Microscopic studies of the atrophic cells in senile macular degeneration patients (post mortem) show retinal pigment epithelium cellular elements are destroyed with the pigment being clumped and adhered to Bruch's membrane. These studies suggest an inflammatory process induced by a degradation product or irritant in the area of the destroyed retinal cells. That is why the Macular degeneration of the retina is a progressive degeneration of the pigmented cells and subsequent destruction of the cone photoreceptors of the retina of unknown etiology.

Interestingly, the retina has a similar topographical layer arrangement of cytoarchitecture to the brain. The six layers of the retina carry the function of transmitting light stimuli into the brain through the optic nerve. Then through the brainstem structure of the lateral geniculate, the optic radiates to the occipital lobe sensory neurons. The layers of the retina consists of a neuro-ectodermal layer of rods and cones, an intermediate layer of bipolar cells, horizontal cells and Muller's cells, and the inner layers containing ganglion cells, glia, nerve fibers, and internal limiting membrane separated from the choroid by retinal pigment epithelium (RPE).

The rods and cones are the photoreceptors. They consist of photoreceptive pigment and inner segments with dense packing of mitochondria. Besides retina, the pigmented cells occur in the red nucleus, substantia nigra, and locus coeruleus in the brain. These pigmented cells of the retina are hexagonal cells lying just externally to the rods and cones layer of the retina. These cells provide insulation of melanin pigment, nutrition and provide the Vitamin A substrate for the photosensitive pigments in the rod and cone cells.

Patients with an early stage of ARMD are diagnosed by the occurrence of anomalous clumps of irregular pigments in the eye examination namely Drusen (FIG. 2). The first visible defect in ARMD is buildup of drusen, a lipoproteinaceous deposit between RPE and Bruch's membrane, the extra cellular matrix between the RPE and the underlying choroid. Drusen are a significant risk factor for the progression to choroidal neovascularization (CNV), the most important cause of vision loss in ARMD (FIG. 2). The presence of large, soft drusen (FIG. 2) in the eye indicates a pre-stage of exudative ARMD, and places patients at higher-than-average risk for developing neovascularizations (FIG. 2).

As noted, the loss of central vision in macular degeneration is due to the atrophy of the retinal pigment epithelium associated with loss of cone retinal photoreceptors. There have been reports of histiocytes and giant cells in the areas of breaks in Bruch's membrane (which acts as blood retinal barrier) and subretinal neovascular membranes. The RPE transports metabolic waste from the photoreceptors across Bruch's membrane to the choroid. Bruch's membrane gets thicker (up to 3 times the normal) with advancing age. This impedes the transportation of waste material which can cause a buildup of deposits and contribute to ARMD phathophysiology.

The development of drusen may be the result of this clogging of the transport system. These built up deposits are called: 1. Basal Linear Deposits or BLinD and 2. Basal Lamellar Deposits or BLARMD. These deposits are formed on and in Bruch's Membrane. The deposits cause breakdown of this membrane and allows the choroid vessels to burst through and to expand into the membrane and RPE where it is beyond the retina itself. In choroidal neovascularization (CNV), capillaries coming from the choroid must cross Bruch's membrane to reach the subretinal pigment epithelial space. Studies show that the “Human Bruch's membrane ages like arterial intima” and the plasma lipoproteins are the known source of extracellular cholesterol. Hence the “Age-related maculopathy and atherosclerotic cardiovascular disease may share joint pathogenic mechanisms”

The retina is supplied by two vascular layers. Retinal vessels from the central artery of the retina (a branch of the ophthalmic artery) supply the inner two-thirds. The outer retina is completely avascular which receives oxygen and nutrients from the choroidal BV. To enhance transport of oxygen and nutrients and to remove the metabolites from the photoreceptors, there is a major pool of fenestrated choroidal capillaries beneath the retina. This pool is referred to as the choriocapillaris.

Plasma and other constituents leak out of the choriocapillaris to pools beneath the retinal pigmented epithelium (RPE), which has tight junctions with several transport systems. This constitutes the outer blood-retinal barrier through the Bruch's membrane. Inner Retinal vascular endothelial cells have tight junctions which creates the inner blood-retinal barrier. The inner limiting membrane (ILM) lines the inner surface of the retina and the peripheral borders of the vitreous, which is also avascular. The inner retina is a vascularized tissue sandwiched between two avascular tissues which the outer retina is an avascular tissue pack in between two vascularized tissues.

The unique architecture of the retina makes the possibility to clearly identify two types of neovascularization: First, retinal neovascularization, which sprouts from retinal vessels, penetrates the Inner Liming Membrane (ILM) and grows into the vitreous (although, under some circumstances, the vessels grow the other way through the avascular outer retina to the subretinal space). Second, Choroidal Neochoriocapillares, which sprouts from choroidal vessels, penetrates Bruch's membrane and grows in the sub RPE and subretinal spaces (FIG. 2) (Campochiaro P. A., Retinal and Choroidal Neovascularization, journal of cellular Physiology 184:301-310, 2000).

Blood vessels develop by vasculogenesis, angiogenesis, or intussusception. During vasculogenesis, the endothelial cells of the BV differentiate from precursor cells and angioblasts which are already present throughout the tissue, where there is linkage in concert to form vessels. During angiogenesis, BV germinates from preexisting BV and invades into surrounding tissue that we see in ARMD (FIG. 2). Most organs are vascularized by vasculogenesis, except, the brain and parts of the kidney. Retinal vascular development occurs by a combination of vasculogenesis (new BV) and angiogenesis (McLeod D S, Lutty G A, Wajer S D, Flower R W. 1987. Visualization of a developing vasculature. Microvasc Res 33:257-269. McLeod D S, Crone S N, Lutty G A. 1996. Vasoproliferation in the neonatal dog model of oxygen-induced retinopathy. Invest Ophthalmol V is Sci 37:1322-1333.). Superficial retinal vessels formed by vasculogenesis.

Angiogenesis plays an important role in pathogenesis of wet ARMD and many eye diseases and other systemic diseases including cancers. Hence, it is important to understand the pathophysiology of this process, to understand the effect of various pharmacological and therapeutic anti angiogenesis agents for the treatment of ARMD. U.S. Pat. No. 6,525,019 B2 discloses melanin based therapeutic agents for inhibition of angiogenesis of ARMD.

Angiogenesis is the most common cause of blindness and is involved in approximately twenty eye diseases. Such angiogenic damage is associated with diabetic retinopathy, retinopathy of prematurity, corneal graft rejection, neovascular glaucoma, and retrolental fibroplasias, ARMD etc. The only known angiogenesis inhibitors which specifically inhibit endothelial cell proliferation are angiostatin protein and Endostatin™ protein (O'Reilly M. S., Holmgren L., Shing Y., Chen C., Rosenthal R. A., Cao Y., Moses M., Lane W. S., Sage E. H., Folkman J. Angiostatin: a circulating endothelial cell inhibitor that suppresses angiogenesis and tumor growth. Cold Spring Harbor Symp. Quant. Biol., 59: 471-482, 1994. O'Reilly M. S., Boehm T., Shing Y., Fukai N., Vasios G., Lane W. S., Flynn E., Birkhead J. R., Olsen B. R., Folkman J. Endostatin: an endogenous inhibitor of angiogenesis and tumor growth. Cell, 88: 277-285, 1997. Yoon S. S., Eto H., Lin C. M., Nakamura H., Pawlik T. M., Song S. U., Tanabe K. K. Mouse endostatin inhibits the formation of lung and liver metastases. Cancer Res., 59: 6251-6256, 1999. Dhanabal M., Ramchandran R., Waterman M. J., Lu H., Knebelmann B., Segal M., Sukhatme V. P. Endostatin induces endothelial cell apoptosis. J. Biol. Chem., 274: 11721-11726, 1999.). Thus, the new methods and compositions are needed that are capable of inhibiting angiogenesis and treating angiogenesis-dependent diseases like wet ARMD and the other angiogenesis related diseases of the eye and other parts of the body. Such antiangiogenesis effect is augmented—amplified by the use of our invention in conjunction.

Individuals with lighter iris color have been found to have a higher incidence of age related macular degeneration (ARMD) than those with darker iris color. Lighter eye color is coupled with an increased risk of ARMD progression (Frank R N, Puklin J E, Stock C, Canter L A (2000). “Race, iris color, and age related macular degeneration”. Trans Am Ophthalmol Soc 98: 109-15; discussion 115-7). Evidence indicates that individuals with increased iris pigmentation have a decreased risk of developing ARMD. The increased levels of eumelanin appear to be more protective than pheomelanin and the light-absorbing characteristics of melanin which are thought to be responsible for this protective effect (Hammond B R, Jr, Fuld K, Snodderly D M. Iris color and macular pigment optical density. Exp Eye Res. 1996; 62:293-297).

An alternative hypothesis is that increased levels of melanin may protect against age related increases in lipofuscin (implicated in photo-oxidative mechanisms). However, these prior studies do not teach, discuss, or suggest the antiangiogenic ability of melanin to inhibit blood vessel growth and macular degeneration, as disclosed in the invention U.S. Pat. No. 6,525,019 B2.

According to the present invention, melanin, or a melanin-promoting compound, may be used in combination with other compositions and procedures for the treatment of diseases such as ARMD. The melanin, or melanin-promoting compound, formulations includes those suitable for oral, ophthalmic (including intravitreal or intracorneal or conjunctival sac), nasal, topical (including buccal and sublingual), and other parenteral routes.

U.S. Pat. No. 6,936,043 B2 and U.S. Pat. No. 6,942,655 B2 disclose using PDT to treat ARMD and may need many treatments which can further damage the retina. PDT prevents or alters the function of the neovascular tissue by using low energy light to generate reactive species within the vessels, or within and around the vessels, to thereby damage these vessels and prevent further growth.

U.S. PATENT APPLICATION PUB. NO.: 2003/0065020 AI discloses a method of treating or preventing macular ARMD by administering an HMG-CoA reductase inhibitor. It was based on the finding that men and women who use statins are associated with an 11-fold reduction in risk of macular degeneration. Statins are inhibitors of 3-hydroxy-3-methylglutaryl coenzyme A, i.e. HMG-CoA reductase inhibitors. Accordingly, we provide that age related macular degeneration (ARMD) is effectively treated by administration of HMG-CoA reductase inhibitors like statins comprising: fluvastatin (Lescol), cerivastatin (Baycol), atorvastatin (Lipitor), imvastatin (Zocor), pravastatin (Pravachol), lovastatin (Mevacor) and rosuvastatin (ZD 4522). They provide a method of treating ARMD by: (a) lowering the level of LDL cholesterol in the patient; (b) increasing the level of HDL cholesterol in the patient; and (c) lowering the level of triglycerides in the patient's blood.

Other HMG-CoA reductase inhibitors are disclosed in U.S. Pat. No. 6,218,403, U.S. Pat. No. RE 36,481 and U.S. Pat. No. RE 36,520 U.S. Pat. Nos. 5,877,208, 5,792,461 and 5,763,414 disclose the use of naringin and naringenin, citrus peel extract and hesperidin and hesperetin respectively as HMG-CoA reductase inhibitors. These can be incorporated with our invention of insulin to treat ARMD.

U.S. Pat. No. 6,218,403, U.S. Pat. No. RE 36,481 and U.S. Pat. No. RE 36,520 U.S. Pat. Nos. 5,877,208, 5,792,461 and 5,763,414 discloses a method of treating age related macular degeneration with a therapeutic amount of a prostaglandin F₂ₐ from derivative like latanoprost. This method is based on the property of prostaglandin F₂ₐ derivatives which these derivatives cause the iris and other tissues to darken when applied topically to the eye. This may increase the melanin and reduce the ARMD when used in conjunction with our invention topically.

A novel process for making latanoprost is taught in U.S. Pat. No. 5,466,833 and the use of latanoprost in treating glaucoma are disclosed in U.S. Pat. No. 5,510,383. It is known that prostaglandin F derivatives have the ability to stimulate melanogenesis in tissues which they are applied as described in U.S. Pat. No. 5,905,091. The application of latanoprost to the eye during the treatment of glaucoma results in increased pigmentation of the eye when light-colored eyes with blue irises can change to brown irises. This effect of prostaglandin F₂ₐ derivatives is discussed in the drug insert for the latanoprost ophthalmic solution from Pharmacia & Upjohn. This melanogenistic Property has been seen as a negative side effect of the use of prostaglandin F₂ₐ derivatives. It is suggested treatment be discontinued if increased pigmentation ensues during treatment. Solutions to overcome this problem are disclosed in U.S. Pat. No. 5,886,035. In ARMD, the melanogenesis factor is taken as positive to restore the function of the RPE and treat ARMD.

U.S. Pat. No. 6,525,019 B2 discloses the therapeutic agent melanin for inhibition of angiogenesis of ARMD. Melanin located within specific cells called melanocytes. Melanins are present in the skin, hair and eyes where they impart the color and play a role in light absorption which acts as free-radical scavenger (antioxidant).

U.S. Pat. No. 2,145,869 by Dr. Donato Perez Garcia disclose a method for the treatment of syphilis in general and neurosyphilis in particular using subcutaneous insulin injections followed by intravenous infusion of arsenic, mercury, and bismuth, therapeutic agents with glucose and calcium chloride.

U.S. Pat. No. 4,196,196 discloses a composition of insulin, glucose and magnesium dipotassium ethylene diamine tetra acetic acid (EDTA) to enhance tissue perfusion and to facilitate a divalent/monovalent cation gradient uptake in and out of the cells. Insulin in the intravenous infusion with glucose enhances the uptake and activity of potassium and magnesium at the extra and intra cellular level which is well established.

I have used this method for decades in many surgical and post surgical patients that have other diseases to alter the potassium level in the extracellular fluid (blood) and intracellular levels of the cells, whenever, there was low or high levels of potassium in the serum.

U.S. Pat. No. 4,971,951 and U.S. Pat. No. 5,155,096 discloses Insulin Potentiation Therapy (IPT) for the treatment of virally related diseases such as hepatitis and AIDS, Gonorrhea, duodenal ulcer, gall stones, epilepsy, schizophrenia, asthma, arthritis, osteomyelitis, cancers, and many other disease conditions using insulin. These inventions do not describes the use of insulin and/or IGF-1 locally to treat age related macular degeneration or any other oculopathies or other local disease condition of the other organs as described in this invention. None of these inventors and patents discloses or describes the local (topical) or regional tissue or organ specific use of insulin and/or IGF-I in a restricted area of the tissue or organ to treat the disease states described here in for treating age related macular degeneration.

### SUMMARY OF THE INVENTION

A method for treating age related macular degeneration uses insulin applied topically in a therapeutically effective amount to an affected conjunctival sac. The therapeutically effective dose is delivered to the fovea centralis and macula lutea. In other embodiments, additional therapeutic, pharmaceutical, biochemical, nutriceutical, biological agent or compound, and organic and inorganic agents are also applied to the afflicted site. IGF-1 may be applied as well.

The present invention is for a method of instilling insulin ophthalmic drops in the conjunctival sac for treating Age related macular degeneration due to any etiological factors. The age related macular degeneration is treated with Insulin and/or IGF-I with or without known anti-age related macular degeneration therapeutic, pharmaceutical, biochemical, and biological agents or compounds, nurticeuticals, and drugs.

The present invention furthermore uses this method as a prophylactic on patients where the patients are predisposed to develop Age related macular degeneration. The present invention additionally relates to treatment of other oculopathies associated with and/or contributing to age related macular degeneration.

Accordingly, the present invention provides for the use of therapeutic agents for unexpected pathological state of the retina and to maintain its health and integrity without vision loss.

The present invention uses insulin to stimulate the retinal pigment epithelium to maintain proper functioning of the retina and Bruch's membrane.

The current invention uses insulin to stimulate the Bruch's membrane to function properly, maintain its' integrity to prevent the growth of choroidal capillaries into RPE, and to act as effective retinal-choroid barrier.

The present invention uses insulin in its various forms to induce mitogenesis of stem cells in the retinal complex and maintain the health of the retina.

The present invention uses IGF-I to stimulate the retinal pigment epithelium to maintain proper functioning of the retina with monoclonal antibodies.

The present invention uses IGF-I to stimulate the Bruch's membrane to function properly, and to maintain its' integrity, which prevents the growth of choroidal capillary in the RPE, and to act as effective retina-choroid barrier.

The current invention uses IGF-I in its various forms to induce mitogenesis of stem cells in the RPE and retinal complex, and to maintain the health of the retina and its receptors cells.

The present invention uses insulin and IGF-1 to stimulate the retinal pigment epithelium to maintain proper functioning of the retina.

The present submitted invention uses insulin and/or IGF-1 to enhance and cause angiogenesis, and to increase the blood supply to the retina which will stop the development of abnormal blood vessels.

The present described invention uses platelet derived growth factor (PDGF) along with insulin and/or IGF-1 to enhance the cell growth in retinal pigment epithelium and retinal receptors.

The present invention uses deferoxamine to increase the normal angiogenesis by chelating the iron content in the choroid and prevents the development of abnormal blood vessels as seen in ARMD.

The present invention uses melanin or melanin promoting compounds to prevent the angiogenesis along with insulin and IGF-1 to enhance their therapeutic activity

The present invention uses therapeutic agents such as verteporfin, protoporphyrin, SnET2, Npe6, ATX 06, ICG, etc along with insulin and IGF-1 factors to prevent the formation and to enhance the destruction of abnormal chodriocapillaries.

The present invention uses collagenase inhibition properties, antioxidant activity, inhibition of protein synthesis in rapidly dividing cells, and perturbation of leukocyte functions. The interference with lymphocyte proliferation and anti-inflammatory effects” of tetracycline and its derivatives, rifamycin and its derivatives, macrolides, and metronidazole, with insulin and IGF-1 factors prevents the formation and the destruction of formed capillaries.

The present invention uses various prostaglandin derivatives (such as Latisse already approved for eye lash growth and glaucoma) with insulin and/or IGF-I to enhance production of melanin in the RPE. This maintains its integrity and prevents the permeation of newly formed choriocapillaries.

The present invention uses antioxidants such as curcumin, vitamin E, D₃, A—precursors and derivatives—, omega 3 along with insulin and/or IGF-I to enhance health of the RPE, Retina, Bruch's membrane and chodriocapillaries.

The present invention also uses additional growth factors known to promote RPE, Bruch's membrane, retina and choroid (besides insulin, insulin-like growth factor—IGF-1), such as interleukin-4 (IL-4), transforming growth factor (TGF—e.g., TGFα or TGFPI), basic fibroblast growth factor (bFGF), epidermal growth factor (EGF), platelet-derived growth factor (PDGF), Vascular endothelial growth factor (VEGF), metformin, vitamin K or biotin to enhance the health of the RPE, Retina and chodriocapillaries.

The present invention uses HMG-CoA reductase inhibitor with insulin and/or IGF-I to enhance health of the RPE, retinal photoreceptors, and choriocapillaries and to prevent the accumulation of athermanous material in any these delicate eye structures.

The present invention desensitizes the body's response to its own innate hormones using progesterone's with insulin and/or IGF-I to enhance health of the RPE, Retina and chodriocapillaries.

The present invention discloses a method of administering an effective amount of a combination of polyvinyl pyrollidone (PVP), procaine and thiamine to a mammalian host with insulin and/or IGF-I to enhance health of the RPE, Retina and chodriocapillaries.

The present invention administers an effective amount angiotensin converting enzyme inhibitors with insulin and/or IGF-I to enhance health of the RPE, Retina and chodriocapillaries.

The present invention discloses a method and apparatus for effectively administering a natural enzyme lipase (lipoprotein lipase) into the posterior sclera in close proximity to the macula that will dissolve lipid deposits in the body of the membrane and assist in their removal through the choroidal circulation, along with insulin and/or IGF-I to enhance health of the RPE, Retina and chodriocapillaries.

The present invention is used with all forms of wet, age-related macular degeneration by administration of an anti-vascular endothelial growth factor (anti-VEGF) compound along with insulin and/or IGF-I to enhance health of the RPE, Retina and chodriocapillaries.

The present invention uses medication comprising lutein (wherein the carotenoid is lutein and/or zeaxanthin) and/or zeaxanthin and/or certain antioxidants (or a mixture thereof) that are tailored to an individual by providing an effective amount of a carotenoid and/or vitamin C, vitamin E; beta carotene, zinc and or copper, and/or a mixture thereof (the AREDS Cocktail) to said subject, with insulin and/or IGF-I to enhance health of the RPE, retina and chodriocapillaries.

The present invention is used to treat all forms of wet, age related macular degeneration by administering topiramate with a pharmaceutically effective dosage to suppress degeneration or induce growth of new optic nerve fibers over a sustained period along with insulin and/or IGF-I to enhance health of the RPE, Retina and chodriocapillaries.

The present invention is for use with all forms of wet, age related macular degeneration by the administration of a topical application of non-steroidal anti inflammatory agents (NSAID) along with insulin and/or IGF-I to enhance health of the RPE, Retina and chodriocapillaries.

The present invention is for use with all forms of wet, age related macular degeneration by administration of an topical application of carbonic anhydrase inhibitors to the eye such as dorzolamide, acetazolamide, methazolamide and other compounds along with insulin and/or IGF-I to enhance health of the RPE, Retina and chodriocapillaries.

The present invention is for use with all forms of wet, age related macular degeneration by administration of a topical application of with a therapeutic amount of a prostaglandin F₂ₐ, derivative such as latanoprost along with insulin and/or IGF-I to enhance health of the RPE, Retina and chodriocapillaries by increasing the melanin content which is antiangiogenic.

The present invention is for use with all forms of wet, age related macular degeneration by administration of an topical application of a method of inhibiting angiogenesis in an individual comprising administering to an individual an angiogenesis inhibiting amount of melanin, inhibiting amount of a melanin-promoting compound such as latanoprost or salts of aminoimizazole carboxamide and CAI triazole which have antiangiogenesis effects along with insulin and/or IGF-I to enhance health of the RPE, retina and chodriocapillaries.

One embodiment of the present invention uses insulin and/or IGF-I mixed with a mucosally compatible vehicle or carrier with proper pH which may be employed for preparing compositions of this invention. For example, aqueous solutions are e.g., physiological saline, ringers lactate, dextrose, oil, solutions or ointments, and dimethyl sulfoxide. The vehicle may contain mucosally compatible preservatives such as e.g., benzalkonium chloride, surfactants like e.g., polysorbate 80, liposomes or polymers. For example, methyl cellulose, polyvinyl alcohol, polyvinyl pyrrolidone, and hyaluronic acid may be used for increasing the viscosity. Furthermore, it is possible to prepare compounds mixed with transmucosally compatible absorption enhancers, antibacterial agents, blood vessel dilators, and anti allergic compounds.

Other features and advantages of the instant invention will become apparent from the following description of the invention which refers to the accompanying drawings.

### BRIEF DESCRIPTION OF THE DRAWINGS

FIG. 1 is a schematic view of the longitudinal section of the eye and the location of the macula lutea and its histological structures 106-112 affected in the ARMD of the macula.

FIG. 2 is a schematic view of the longitudinal section of the part of the eye and the location of the macula lutea and its histological changes in ARMD compared to healthy retina.

FIG. 3 is a diagrammatic presentation showing the conjunctival fornix and the route of drainage of therapeutic agents to the nose.

### DETAILED DESCRIPTION OF THE INVENTION

In the following detailed description of the invention, reference is made to the drawings in which reference numerals refer to like elements, and which are intended to show by way of illustration specific embodiments in which the invention may be practiced. It is understood that other embodiments may be utilized and that structural changes may be made without departing from the scope and spirit of the invention.

Our invention involves the treatment of etiology, physiology, pathology, signs and symptoms of a variety of eye diseases that grouped under the umbrella of ARMD as discussed above and below.

Age Related Macular Degeneration (ARMD): The terms “macular degeneration”, “age-related macular degeneration” and “age related maculopathy”, as well (as the abbreviations “ARMD”, “AMD”, “ARM”) are synonymous with each other.

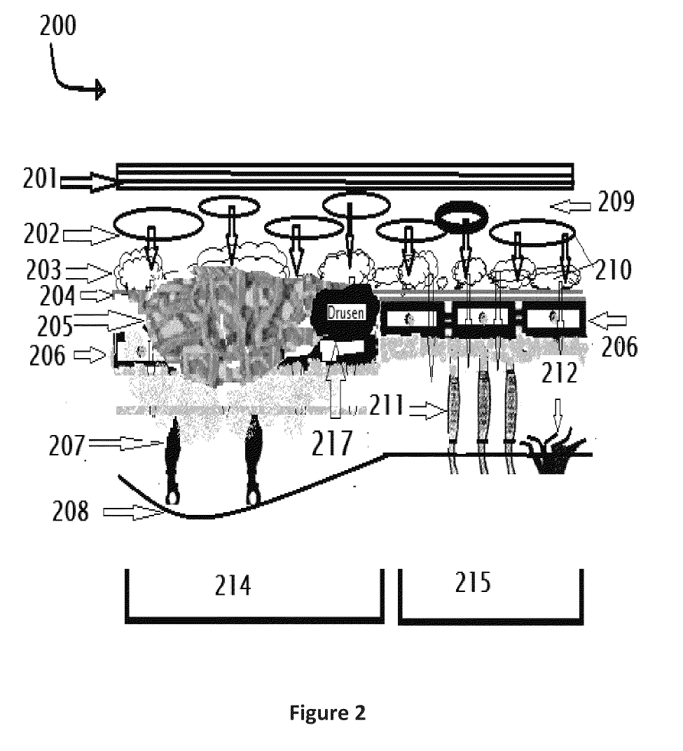

ARMD is an acquired retinal disorder distinguishes by any of the following optic fundus changes on ophthalmic examination: pigment layer atrophy and degeneration, various types of drusen and lipofuscin deposits, and exudative elevation of the outer retinal complex (FIG. 2) in the macular area due to neovascularization, exudation, or bleeding. It occurs in patients over age 55, resulting in progressive, sometimes irreversible loss of central visual function from either fibrous scarring or diffuse, geographic atrophy (pigment epithelium) of the macula. ARMD includes extrafoveal lesions that would have an impact on vision if superimposed on the foveal region (Bressler S B, Bressler N M, Fine S L, et al. Natural course of choroidal neovascular membranes within the foveal avascular zone in senile macular degeneration. Am J Ophthalmol 1982; 93:157-163, Bressler et al, 1988, Surv Ophthalmol 32:375-413).

Nonexudative (dry or atrophic) macular degeneration accounts for 90 percent of ARMD degeneration in the US. This is due to a gradual breakdown of the retinal pigment epithelium (RPE), the accumulation of drusen deposits, and loss of function of the overlying photoreceptors resulting in gradual, progressive loss of central visual function to cause vision levels of 20/200 or worst. The choroidal and subretinal or sub-retinal pigment epithelium exudations are apparently absent in this category of macular degeneration.

Exudative (Wet) Macular Degeneration account for 10 percent of ARMD and contribute to 90 percent of the ARMD patients with considerable vision loss. Exudative macular degeneration is characterized by the development of neovascularization in the choroid, leading to serous or hemorrhagic seepage and subsequent elevation of the retinal pigment epithelium and/or neurosensory retina (FIG. 2). These patients notice great and rapid decrease in central visual function. The leakage from the new choroidal vessels can cause dysmorphopsia, scotoma, and blurred vision.

In the majority of patients, nonexudative macular degeneration will not progress to severe vision loss. When it progresses to the exudative form, the patients are at greatest risk for severe visual destruction. The patients who have exudative maculopathy with drusen are at major risk of developing choroidal neovascularization and vice versa.

Drusen, an indicator of development of future ARMD, are yellowish-white nodular deposits found in the deeper layers of the retina. They comprise hyaline deposits or colloid bodies of Bruch's lamina of the choroid, and may not always affect the vision. Drusen are seen as a consequence of aging which can be found in the younger age group also. Drusen are time and again associated with ARMD with increased risk of visual loss. Drusen may vary in number, size, shape, degree of elevation, and extent of associated changes in the RPE. More often than not occurring in clusters, drusen can be found anywhere in the posterior pole of the retina. In some patients, drusen may be restricted to the region of the fovea, where others deposits encircles the fovea, which spare the fovea, itself. Drusen can appear external to the vascular arcades and are found on the nasal side of the optic disc. Several kind of drusen such as hard and soft mixture has been described: Drusen may gradually enlarge and coalesce pushing the photoreceptors (FIG. 2). Basal Laminar Drusen seen in younger people, are many, small, unvarying, round, subretinal nodules compared to large clumps seen in the aged. Calcified Drusen have a glistening appearance secondary to calcification.

Geographic Atrophy is a clinical manifestation of progressive atrophy of the retinal pigment epithelium in combination with drusen formation. There are several well-demarcated areas of retinal pigment epithelium atrophy go together with by overlying photoreceptor damage. Single or multiple areas of atrophy spread throughout the foveal and the parafoveal area which produces a gradual decrease in vision. Choroidal neovascularization (CNV) can develop as a separate entity in the presence of soft and confluent drusen. Geographic atrophy can follow the collapse of a retinal pigment epithelial detachment. Geographic atrophy can occur after an RPE tear and can be associated with ill-defined or occult choroidal neovascular membranes.

Retinal Pigment Epithelium Abnormalities are considered the earliest retinal manifestations of macular degeneration and consist of increased retinal pigmentary degeneration where atrophy is in the plane of the retinal pigment epithelium. A grayish-yellow or pinkish-yellow area in the macula is surrounded by a halo of gray or black pigment clumps in or beneath the retina. Increased lipofuscin in the retinal pigment epithelium and the accumulation of debris on and within Bruch's membrane results in the loss of photoreceptor function.

Detachment of the retinal pigment epithelium can be an extra symptom exhibited by patients with ARMD seen sharply circumscribed, varying size, and dome-shaped elevation at the posterior pole of the eye (FIG. 2). Fluorescein angiography shows free fluorescein pools in the sub-RPE space giving rise to an area of hyper fluorescence marking the area of retinal pigment epithelial detachment. Patients show signs of RPE detachment which may result in spontaneous resolution, geographic atrophy, detachment of the sensory retina, and development of occult choroidal neovascularization, and the tear of the RPE.

Choroidal neovascularization is the proliferation of fragile; recently formed blood vessels begin in the choroidal space and penetrating through Bruch's membrane and RPE to the outer retinal complex into the subretinal and retinal tissue. Serous or hemorrhagic leakage from these vessels results in a neurosensory or retinal pigment epithelial detachment

Diffuse thickening of Bruch's membrane, in mixture with soft, confluent drusen and pigment abnormalities, predisposes the patient to the development of a choroidal neovascular membrane. The new vessels of the choroidal neovascularization (angiogenesis) form an organized fragile vascular system. As the system matures, the delicate neovascular branches leak fluid (protein, lipids and inflammatory cells) into the subretinal, intraretinal, or sub-retinal pigment epithelium space. Depending on various factors, the hemorrhage at the site of the membrane or in the subretinal space may extend into the vitreous.

Vitreous hemorrhage is able to occur with exudative macular degeneration with sudden vision loss. It is sometimes the result of a breakthrough hemorrhage. The vitreous hemorrhage clear in 75% percent of patients.

A yellowish-white to brown or black lesion is observed in the macula as fibro vascular disciform scar signify the concluding stage of untreated choroidal neovascularization. Subretinal fluid or fresh hemorrhage appears at the edges of the scar with or without hypertrophic retinal pigment epithelium, chorioretinal folds and anastomosis of the retinal and choroidal circulations.

Numerous systems have been proposed for classifying the various stages of macular degeneration based on ophthalmoscopic appearance macular lesions, the extent of involvement of the macula, and the patient's visual acuity and/or “early” or “late” forms. We do not want to go into the details of the classifications. Our invention treats all forms and early to late stages of the ARMD that is described above as well as used as prophylactic against development of ARMD.

One of the most important aspects of our invention is the use of insulin or Insulin like growth factor (IGF-1) when used alone or in combination with or without known ARMD therapeutic agents. The use is for prophylactic measures or treatment of the disease in humans and animals. We discuss our invention insulin and the effectiveness for treating a variety ARMD as facilitators, carriers, adjuvant agents, absorption enhancers to assist to get entry into the cell, to potentiate the therapeutic agent action, the cell metabolic activity enhancers, the cell multiplication enhancers, and to replace the apoptotic cells with healthy cells. Our invention insulin and/or IGF-I are used to enhance the absorption or to potentiate (augmentation-amplification effects) the effect of therapeutic agents administered to the patients for treatment of ARMD and other oculopathies.

The ophthalmic drops or preparations to be used to treat age related macular degeneration should be stable, dissolved or solubilized which the preparation is safe and effective with ophthalmological standards in place. The term ‘stable’, means physical, rather than chemical stability with no crystallization and/or precipitation in the compositions, when the preparation is stored at a refrigerated or room temperature. The preparation comes in contact with lacrimal secretions when the preparation is applied to the conjunctival sac and the cornea. The label ‘dissolved’, ‘dissolving’, ‘solubilized’ or ‘solubilizing’, means that an ingredient is substantially solubilized in the aqueous composition without the particulate, crystalline, or droplet form in the composition.

The phrase ‘ophthalmological acceptable’ refers to those therapeutic, pharmaceutical, biochemical and biological agents or compounds, materials, compositions, and/or dosage forms suitable for use in a mammalian eye without undue toxicity, irritation, allergic response, or other problem or complication, commensurate with a reasonable benefit/risk ratio. The expression ‘safe and effective’ means a concentration and composition that the concentration and composition is sufficient to treat without serious local or systemic side effects. Our invention fulfills all these parameters to be used with ophthalmic drops to treat ARMD. The term “ocupopathies” means any and all diseases affecting the eye lids, eye ball with retina, optic nerve, choroid, eye ball, and their function.

Any treatment of age related macular degeneration with or without other oculopathies with ophthalmic topical preparations (eye drops) designed in our invention using Insulin and/or IGF-1 and other therapeutic agents as prophylactic, and/or for treatment encompass the following principles:

1. Eye drops, semi liquids, gels or ointments should act like a film covering like natural tears over the ocular surface of the eye including cornea with less stinging or burning sensation,

2. The above are capable of providing mechanical lubrication for the ocular surface which the eye lid glides easily during the blinking movement.

3. The reduction of the evaporating natural lacrimal fluid,

4. The emulsion or the watery ophthalmic drops shouldn't react with eye cellular structures, the lacrimal coating, and the eye lid lacrimal glandular system,

5. Eye drops should be stable for a reasonable period of time at room temperature.

6. The therapeutic preparations should be easily absorbed with or without other absorption enhancers and transported to the site of the pathology.

7. Besides acting against age related macular degeneration pathology, the therapeutic preparations should contain therapeutic, pharmaceutical, biochemical and biological agents or compounds capable of alleviating the underlying cause responsible for ARMD; at the same time augments and amplifies the effects of therapeutic agents with trophic effects when used with our invention.

8. The ophthalmic therapeutic agents should have therapeutic healing effects on other oculopathies, which are specific for eye disease, that it is used.

In our invention, insulin based ophthalmic preparations meet all the above recited physiological, pharmacological, and therapeutic parameters.

Conjunctival sac administration of known therapeutic agents, as well as other pharmaceutical, biochemical, nurticeuticals and biological agents or compounds of biologics when compared to systemic administration, carries the following advantages:

1) superior efficacy due to the achievement of higher local concentration at the site of ARMD;

2) greater efficacy due to the ability and ease of therapeutic molecule to reach the target tissue without degradation caused by gastrointestinal, hepatic or systemic circulation;

3) more rapid onset of action due to closeness of the therapeutic agents deposition;

4) longer duration of action due to therapeutic agents stasis at the local site;

5) Possible fewer side effects, due to lower dosage deposited in the conjunctival sac;

6) ease of administration and greatly improved efficacy due to improved delivery with increased compliance of the therapeutic molecule close to the site of pathology—i.e. Retina;

7) Clinical experience utilizing conjunctival sac route of administration of Therapeutic agents with insulin for treating AMRD and other oculopathies has demonstrated the dramatic efficacy, and the remarkable rapid onset of action produced by this route of administration.

Before, the explanation and the description of the disclosed embodiments of the present invention in detail, which it is to be understood that the invention is not limited in its application to the details of the particular examples and arrangements shown. Since the invention is capable of other examples and embodiments in treating other oculopathies. The terminology used, herein, is for the purpose of description and without a limitation. Earlier enumerated above and narrated below: this application has been filed in order to disclose: Insulin and Insulin-like Growth factor (IGF-1) have been found to have high therapeutic activity against metabolism of the cells. All its functions including retina and photoreceptors involved in age related macular degeneration. Insulin and/or IGF-I restores the proper physiological functioning of the retina by acting against the etiological factors such as ROS, genetic defects, correcting any mitochondrial metabolic defect, and restoring the membrane stability. It enhances the effectiveness (augmentation-amplification effects) of other therapeutic, pharmaceutical, biochemical, and biological agents or compounds used in the treatment of age related macular degeneration and other oculopathies. Insulin, our present invention, helps to maintain functional and structural integrity of the photoreceptors when they have genetic defects. Furthermore, our invention insulin helps to delay the expression of genetic defects that these genetic defects exist in the photoreceptors which these genetic defects predisposes or causes the age related macular degeneration.

At present, the insulin is exclusively used to treat type I and certain cases of type II diabetes. Our discoveries and inventions describes the use topically (locally) in other disease conditions besides diabetes that includes cancers, dry eye syndrome, glaucoma, prostate diseases, middle and inner ear afflictions, CNS diseases including autism, Parkinson's disease, depression Alzheimer's, to treat hair loss, enhancing eye lashes, activating vaccines, cytokines, Lymphokine, monoclonal antibodies, activating local immune system at lymph nodes, enhancing the local effects of chemotherapeutic agents, in treatment of autoimmune diseases, age related changes of the facial skin, healing of wounds, gum diseases, local infections and multiple local and systemic therapeutic applications.

INSULIN AND ITS BIOLOGICAL EFFECTS ON HEALTHY AND DISEASE AFFLICTED CELLS, PHOTORECEPTORS CELLS IN AGE RELATED MACULAR DEGENERATION. THE ROLE INSULIN PLAYS WITH THE UPTAKE, DISTRIBUTION; AUGMENTATION-AMPLIFICATION EFFECTS OF THERAPEUTIC, PHARMACEUTICAL, BIOCHEMICAL AND BIOLOGICAL AGENTS OR COMPOUNDS ON THESE PHOTORECEPTOR CELLS ARE DESCRIBED HEREIN.

A variety of carriers, adjuvant agents, absorption enhancers, and facilitators, assists to get entry into the cell. The potentiators of therapeutic action (augmentation/amplification effects), cell metabolic activity enhancers, cell multiplication enhancers, and other methods have been used to enhance the absorption and/or to potentiate the effect of therapeutic, pharmaceutical, biochemical, and biological agents or compounds administered to the patients for improving the physiological function, and the treatment of diseases. Discovery of insulin described in this invention is such a biological agent which we give details and elaborate below.

In 1921, Drs. Frederick Banting and Charles Best at University of Toronto physiology department isolated insulin from dog pancreas and tested this on diabetic dogs, successfully lowering the dogs' blood sugar level. On Jan. 11, 1922, Leonard Thompson, a 14-year-old boy who was dying from diabetes, was given the first human experimental dose of insulin. He lived 13 more years and died from pneumonia.

Aspirin, antibiotics, and insulin are the most commonly used therapeutic agents which are known to the public and professional alike. Insulin is a hormone secreted by beta cells of the islets of Langerhans in the pancreas. It has been self administered in the home by the patient or in the office by the physician to treat diabetes. Insulin can be easily obtained by prescription which the insulin can be used for treating age related macular degeneration as described in this invention. There are no reports of using the insulin as a therapeutic agent locally to treat localized diseases such as ARMD or parentarily to treat systemic diseases such as cancers, autoimmune diseases, scleroderma and many other diseases other than diabetes. The present inventor is the first person to experiment with the use of insulin locally for almost a decade to treat many kinds of diseases of various tissues and organs in the body including cancers, and diseases of the ear, eyes, prostate, teeth, gums, CNS, eyes, hair growth, and other such conditions with many known therapeutic, pharmaceutical, biochemical, and biological agents or compounds.

In 1965 Sodi-Pollares et al. for the first time used glucose-insulin-potassium (GIK) solutions to treat patients with acute myocardial infarction. He found that GIK limited infarct size, reduced ventricular ectopy, and improved survival (Sodi-Pollares D, Testelli M D, Fisleder B L. Effects of an intravenous infusion of a potassium-glucose-insulin solution on the electrocardiographic signs of myocardial infarction. Am J Cardiol. 1965; 5:166-81). Insulin benefits the post ischemic myocardium by stimulating pyruvate dehydrogenase activity, which this activity in turn stimulates aerobic metabolism on cardiac and other tissue reperfusion. Exogenous insulin helps to reverse insulin resistance during cardiopulmonary bypass, which the exogenous insulin contributes to increased serum concentrations of free fatty acids and decreased myocardial uptake of glucose which increased myocardial function. Intravenous direct infusions of insulin after coronary artery bypass graft surgery (CABG) have been shown to decrease the levels of free fatty acids and increase myocardial uptake of glucose.

Insulin added to antegrade and retrograde tepid (29° C.) blood cardioplegia during coronary artery surgery has been shown to stimulate aerobic metabolism during reperfusion, preventing lactate release and improving left ventricular stroke work index with the restarting of the heart beating without many arrhythmias. This is the report of using insulin locally on a dynamic large organ, the heart. You can imagine the effect of insulin at cellular level of small structures such as eye, when insulin has profound effect on a massive dynamic organ like the heart! Insulin is especially beneficial for patients with diabetes and acute coronary ischemia (Svensson S, Svedjeholm R, Ekroth R. Trauma metabolism of the heart: uptake of substrates and effects of insulin early after cardiac operations. J Thorac Cardiovasc Surg. 1990; 99:1063-73. Rao V, Mississauga C N, Merrante F. Insulin cardioplegia for coronary bypass surgery [abstract]. Circulation. 1998; 98 (Suppl):I-612). Insulin increases the glutathione synthesis by activating gamma-glutamyl-cysteine synthetase.

The insulin metabolic effects which the insulin reduces both polymorphonuclear neutrophils adhesion due to ROS (reactive oxygen species) can be effective in post perfusion adhesion of white blood cells to ROS with resultant cellular damage and stimulated tyrosine phosphorylation.

Reactive oxygen species (ROS) are speedy uncontrolled molecules that contain the oxygen atom to include oxygen ions and peroxides. They can be inorganic or organic those are highly reactive due to the presence of unpaired valence shell electrons, where the electrons produce hydrogen peroxides, which cause cell damage due to the cell membranes inside and outside the cells by peroxidation. Photoreceptors and other cells are able to defend themselves against ROS damage through the use of superoxide dismutase's, catalases, lactoperoxidases, glutathione peroxidases, and peroxiredoxins.

Small molecule antioxidants such as ascorbic acid (vitamin C), tocopherol (vitamin E), uric acid, polyphenol antioxidants, and glutathione. These play important roles as cellular antioxidants to protect against ROS. The most important plasma antioxidant in humans is uric acid. H₂O₂ induced and stimulated lipid peroxidation was significantly inhibited by insulin pretreatment. Insulin increased redox status by increasing intracellular glutathione (GSH) content in oxidized cells. This reduced the ROS from the cells. The results show that GSH can reverse the effect of oxidation (oxidative free radical damage) on tyrosine kinase activation and phosphorylation. Thus, GSH plays an important role in cell signaling, which confirms the antioxidant activity of insulin to prevent the photoreceptors damage by ROS.

This is a signal that insulin plays an overwhelming role in maintaining homeostasis. Insulin improves cellular physiological function in addition that the insulin augments/amplifies the effects of therapeutic agents when the insulin is used locally as described below in this invention at localized tissue levels, in the cornea, retina, and in the eye ball. Hence, our invention, with local use of insulin alone or with other therapeutic agents, is very effective in treating ARMD and related afflictions of the retina.

Insulin affects the DNA, RNA, and other protein synthesis which results in increased growth by mitosis (Osborne C K, et al. Hormone responsive human breast cancer in long-term tissue culture: effect of insulin. Proc Natl Acad Sci USA. 1976; 73: 4536-4540); enhances the permeability of cell membranes to many therapeutic agents besides glucose, and electrolytes. Insulin helps and facilitates to move the therapeutic, pharmaceutical, biochemical, nurticeuticals and biological agents or compounds, drugs and therapeutic agents molecules from extra cellular fluid (ECF) to intracellular fluid (ICE) meaning from outside the cells to inside the cells which this facilitation can be seen in the use in coronary artery bypass graft (CABG) surgery.

In our studies of the local effects of insulin, the fact is that the growth hormone is ineffective in the absence of insulin. The local use of insulin does not affect the systemic production of growth hormone, glucagon, adrenalin; and other stress related biological hormonal and nor hormonal agents that is seen in systemic hypoglycemia induced by systemic IPT. The insulin with or without growth hormone is one of the most important biological and therapeutic agents to maintain the health and the functions of all the cells including photoreceptors which the photoreceptors are affected in age related macular degeneration.

Insulin and IGFs have properties of tissue growth factors which they have additional well recognized functions as hormones where the hormones regulate growth and energy metabolism at the whole organism level farther away from the site of production (insulin from the islets of pancreas, IGF-1 from the liver). These are well known as key regulators of energy metabolism and growth. In fact, their physiologies as systemic hormones were recognized long before the details of their signaling mechanisms at the cellular level were described. This is why the Insulin and IGF-1s differ from many other regulatory peptides that the peptides are relevant to regulate physiology at both the whole organism level and the cellular level.

For example, the epidermal growth factor (EGF) and platelet-derived growth factor (PDGF) are examples of peptides that these have important local regulatory roles at the cellular and the tissue levels but not farther from the site. There is little evidence to suggest that circulating levels of these growth factors are physiologically significant.

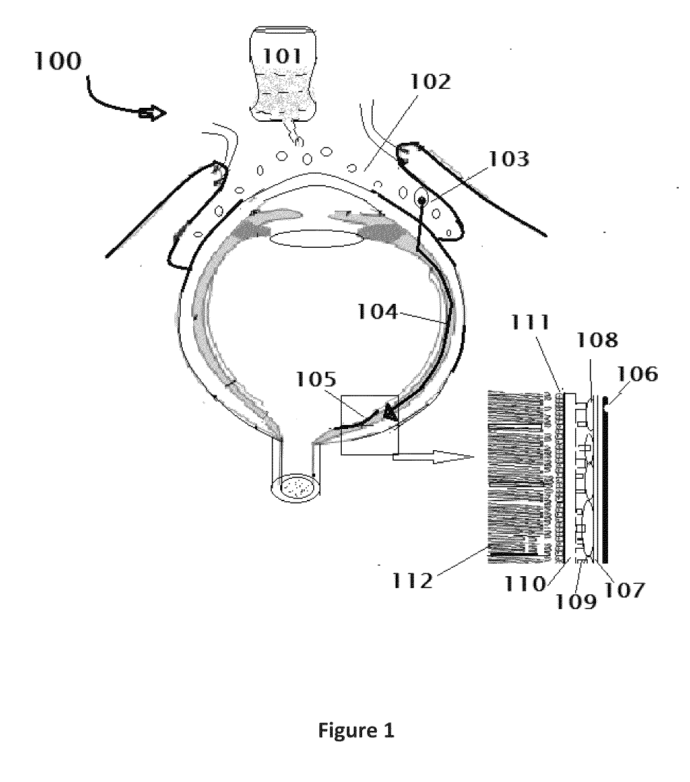

This is the reason our invention with the use of Insulin and IGF-1 topically not only has the local effect. They are absorbed and circulated farther away from the site of the application. They have therapeutic effects on the rods, cones, and in the retina in the age related macular degeneration (Michael Pollak. Insulin and Insulin-Like Growth Factor Signalling in Neoplasia. Nat Rev Cancer. 2008; 8(12):915-928). The IGFs may be an important autocrine, paracrine, or endocrine growth factors. These factors will help to maintain the integrity of photoreceptors when the insulin is transported to the rods and cones of retina from the conjunctival sac (FIG. 1).

Insulin is an anabolic trophic hormone needed for the growth, reproduction, and multiplication of all cells in the body. This includes the healthy vascular endothelium, photoreceptors neurons in the retina (rods and cones), macula, as well as secretory glands of the eye lids including the lacrimal glands (afflicted with Sjogren's syndrome) and the entire eye ball and its contents. The corneal and conjunctival cells which are the cells may be metaplasic in dry eyes syndromes are helped by insulin. Increased cellular metabolic activity induced by insulin enhances the uptake and enhances the action of all therapeutic, pharmaceutical, biochemical, and biological agents or compounds by the cells and inside the cell including the cells responsible or involved in age related macular degeneration.

Insulin enhances the concentration and effectiveness of therapeutic agents which insulin has disease curtailing-curing qualities. Once inside the cells, the insulin augments and amplifies the effects of any and all therapeutic agents including the agent proven and/or approved to treat age related macular degeneration and restoring their physiological function of the rods.

In our decade of studies, medical practice, and experimentation, we found there is not a single disease which cannot be treated except hypoglycemia induced by insulin or otherwise, which a disease cannot be treated using Insulin to enhance the effectiveness of the therapeutic, pharmaceutical, biochemical, and biological agents or compounds including the treatment of age related macular degeneration. In ingenious vitro studies, this has been meticulously and conclusively demonstrated that the insulin activates and modifies metabolic pathways in MCF-7 human breast cancer cells. The insulin increases the cytotoxic effect of methotrexate up to 10,000 (ten thousand) fold (Oliver Alabaster' et al. Metabolic Modification by Insulin Enhances Methotrexate Cytotoxicity in MCF-7 Human Breast Cancer Cells, Eur J Cancer Clinic; 1981, Vol 17, pp 1223-1228. Richard L. Schilsky and Frederick. S. Ordway. Insulin effects on methotrexate polyglutamate synthesis and enzyme binding in cultured human breast cancer cells. Cancer Chemother Pharmacol (1985) 15: 272-277). The data suggests that insulin augmentation of MTX polyglutamate synthesis may account for insulin's previously observed ability to enhance MTX Cytotoxicity (research studies in human breast cancer). My own research studies on every kind of cancer and infection in any part of the body have shown that the group treated with insulin, plus, with low dose methotrexate and other anticancer agents (and/or antibiotics for infection, autoimmune diseases treatments, monoclonal antibody treatment etc.) responded better than the patient treated with insulin or chemotherapy alone (Eduardo Lasalvia-Prisco et al. Insulin-induced enhancement of antitumoral response to methotrexate in breast cancer patients. Cancer Chemother Pharmacol (2004) 53: 220-224. Ayre S G, Perez Garcia y Bellon D, Perez Garcia D Jr (1990) Neoadjuvant low-dose chemotherapy with Insulin in breast carcinomas. Eur J Cancer 26:1262-1263; T. R. Shantha presented at Cancun IPT meeting 2nd meeting 2004 and unpublished studies).

These observations supports the findings of Alabastor (IBID) that the disease or the healthy cell sensitivity to the therapeutic, pharmaceutical, biochemical, nurticeuticals, biological agents or compounds, and drugs that are used to treat age related macular degeneration. This can be increased (augmentation/amplification effects) many times by using the method described in this invention using insulin and/or IGF-I. The effect of insulin in reducing the ROS and other etiological factors in age related macular degeneration is profound.

Our study of injecting Insulin followed by anticancer chemotherapeutic agents directly into cancer masses on hundreds of advanced and localized cancers supports these findings. Using this method, the palpable tumors include enlarged lymph nodes with tumors or tumor deposits that literally disappeared. We treated multiple brain cancer patients by directly injecting insulin with mannitol followed with specific anti tumor chemotherapeutic agents with dextrose where heparin was directly infused into the internal carotid artery with positive results. Patients lived longer with a good quality of life with fewer side effects to the chemotherapy agents.

We have used insulin locally as a therapeutic agent in chronic non-healing wounds, burns, after draining the hydrocele of the tunica vaginalis sac in the scrotum, periodontal diseases, post surgical wound healing, delayed healing of broken bones; prostate and bladder afflictions, teeth and gum afflictions, ear diseases and many other diseases which will be reported at a later date.

The present inventors have used insulin mixed injectate to augment the local anesthetic, or narcotic or steroid effects alone or in combination of the selected therapeutic agents where the agents were introduced into the epidural or subarachnoid space for the treatment of back pain and/or to relieve other kinds of pain due to different etiologies including post operative and cancer pain with excellent rapid, prolonged pain relief (under study).

The present inventors used insulin locally in intravenous regional anesthesia (Bier Block) for surgical procedures of the limbs, pain, to treat reflex sympathetic dystrophy (RDS) and complex regional pain syndrome (CARMDS) mixed with ketamine, insulin and known selected therapeutic agents. Previously, the other methods to treat RSD have been documented with partial success with injectates containing lidocaine, solumedrol and other corticoseroids, bretylium, guanethidine, reserpine, ketorolac, and non-steroidal anti-inflammatory drugs in saline (Neil Roy Connellya, Scott Reubena and Sorin J. Brullb Y. Intravenous Regional Anesthesia with Ketorolac-Lidocaine for the Management of sympathetically-Mediated Pain. Yale Journal of Biology and Medicine 68 (1995), pp. 95-99). We had better success using insulin containing injectates with ketamine with above therapeutic agent's solutions in addition to the injectates which this will be reported at a later date. We had better success using insulin with ketamine delivered directly to the CNS in curtailing and curing complex regional pain syndromes (CARMDS) with reflex sympathetic dystrophy (RSD) & causalgia and many pain related complex neurological disorders.

The word, Prolotherapy, means “PROLO” is short for proliferation because the treatment causes the proliferation (growth and formation) of a new ligament tissue (fibroblasts and collagen formation in the weak, stretched or torn ligaments) in areas where the tissue has become weak which the weakness resulted in pain with movement (Ross A. Hauser, Marion A. Hauser. 2007. Prolo Your Pain Away! Curing Chronic Pain with Prolotherapy. Chicago—Amazon books). Many solutions are used in inducing ligamenotous growth like the use of dextrose (10%-25%) with lidocaine (a local anesthetic 0.1-0.2%), phenol, glycerin, cod liver oil extract, solution containing 1.25% phenol, 12.5% dextrose, 12.5% glycerin, Glucose 20% and Lidocaine 0.1% solution. The mixture of 1 cc of 5% sodium morrhuate and 1 cc of 1% lidocaine, Hackett-Hemwall prolotherapy method of using 15% dextrose, 10% Sarapin (a pitcher plant derivative) and 0.2% procaine solution or Dr. DeHaan's “Prolo Cocktail” containing 25% of each of the following substances: 50% dextrose, 2% lidocaine or procaine (without epinephrine), vitamin B12 (1000 mcg/ml), and Biosode (“a homeopathic with growth and Krebs cycle energy factors”) has been used.

The inventors have used glucose along with insulin, deferoxamine, and lidocaine in prolotherapy injectate for various musculoskeletal pain, including arthritis, back pain, neck pain, fibromyalgia, sports injuries, unresolved whiplash injuries, carpal tunnel syndrome, chronic tendonitis, partially torn tendons, ligaments, and cartilage, degenerated or herniated discs, TMJ pain and sciatica. It is important to note that the principle of prolotherapy is to induce fibroblasts to multiply and to lay more ligaments (collagen).

This method of treatment makes the ligaments and tendons stronger which induces sterile inflammation at the site. Insulin enhances the multiplication of fibroblasts and deferoxamine enhances the angiogenesis to support the multiplication of fibroblasts. Glucose causes sterile inflammatory response and the lidocaine alleviates the pain at the injection site. This combination contributes to the therapeutic effect of prolotherapy to make the ligaments stronger and pain free. The insulin used in the above preparation with the prolotherapy was more effective compared to when the prolotherapy therapeutic agent was used without insulin.

Insulin increased the fibroblast mitosis which increased production of collagen and maintained the integrity of cartilages within the joints to strengthen the ligaments of the painful joint. This gave long lasting rapid pain relief with stronger functional joints when insulin is therapeutically effective in taking away the pain by various prolotherapy therapeutic agents. One can see the effectiveness of the agents in treating the age related macular degeneration and associated diseases of the eye. Besides the insulin, purified platelet growth factor added can promote angiogenesis, increased the blood supply, increased the multiplication of fibroblasts on the ligaments, and torn meniscus, and enhanced the healing process.

The purified genetically engineered platelet growth factors are available to enhance the healing in non-healing bone fractures which the factors can be used to treat the torn meniscus, cartilages and ligaments. Deferoxamine (DFO) is an iron-chelating agent on the formulary that DFO has been shown to increase angiogenesis. We have used Deferoxamine (iron chelator and angiogenesis growth factor, similar to platelet growth factor) and insulin (stimulates metabolic activity and multiplication of cells) sprayed on non healing chronic ulcers with good successes. We have used insulin and Deferoxamine with prolotherapy agents in selected cases which involved ligament tears with joint pain with successfulness. We suspected that some of these cases had meniscus tears. This gave good post therapy results after injecting these therapeutic agents inside the joints on the collateral ligaments with the avoidance of the surgical intervention.

Trigger points or trigger sites are described as hyperirritable spots in skeletal muscle that are associated with palpable nodules in taut bands of muscle fibers where the compression of the fibers or the application of pressure or the contraction of the muscle where the contraction may elicit local tenderness, referred pain, or local twitch response. There are many therapies to take away the tenderness and the sore spots.

Various injections can be used including saline, local anesthetics such as procaine hydrochloride (Novocain); a mixture of lidocaine, and marcaine without steroids (Steroids can cause muscle damage; hence contraindicated) when this is used to relive the pain. Trigger pain point injection for myofacial pain, fibromyalgia, tennis elbow, intercostal pain, wrist and back pains, and injection of joints with therapeutic agents such as local anesthetic with insulin resulted in rapid and effective relief of pain compared to injectate with absence of the insulin.

The palpable nodule of trigger point were reduced or disappeared. The same methods can be used to treat the age related macular degeneration, and any condition contributing to the age related macular degeneration of the eye in combination with other known therapeutic, pharmaceutical, biochemical, and biological agents or compounds as described above.

The examples described above show the effectiveness of the insulin in treating locally disease-afflicted tissue. The same time exert augmentation/amplification effects of therapeutic agents to prevent, delay, curtail and cure the diseases, which the insulin will have the same type of effect in treating age related macular degeneration.

In an important experiment, Zheng et al showed the role of insulin like growth factor-I (IGF-I) that insulin like effects induced the inner ear epithelial cell culture growth (Zheng, J. L., Helbig, C. & Gao, W-Q. Induction of cell proliferation by fibroblast and insulin-like growth factors in pure rat inner ear epithelial cell cultures. J. Neurosci. 17:216-226 (1997). There is a clear indication that insulin and IGF-I not only played a role in potentiation of (augmentation/amplification effects) the therapeutic, pharmaceutical, biochemical and biological agents or compounds. They can independently stimulate cells growth in eye structures (as it happens in the inner ear epithelial cells) including retinal cells particularly photoreceptor cells (Shantha T. R., Unknown Health Risks of Inhaled Insulin. Life Extension, September 2007 pages 74-79, Post publication comments in September 2008 issue of Life Extension, Pages 24. Shantha T. R and Jessica G. Inhalation Insulin, Oral and Nasal Insulin Sprays for Diabetics: Panacea or Evolving Future Health Disaster. Part I: Townsend Letter Journal: Issue #305, December 2008 pages: 94-98; Part II: Townsend Letter, January, 2009, Issue #306, pages-106-110).

The normal cell undergoes the following changes as pathological state takes its root:

1. Dysplasia, where cell maturation and differentiation are delayed, often indicative of an early neoplastic process. The term dysplasia is typically used when the cellular abnormality is restricted to the originating tissue, in the case of an early, in-situ neoplasm. This means that the original cells are not healthy enough to withstand the new environment. The cells changes into another type more suited to the new environment.

2. Metaplasia is the reversible replacement of one differentiated cell type with another mature differentiated cell type. The medical significance of metaplasia is in some sites. The cells may progress from metaplasia, to develop dysplasia, and then malignant neoplasia (cancer).

3. Heteroplasia is the abnormal growth of cytological and histological elements without a stimulus. Insulin has profound effect on these cells undergoing metaplasia and dysplasia. Heteroplasia is indicated in our above articles published in Life Extension and Townsend letters research publications. The changes contributing to the pathology of the eye diseases includes age related macular degeneration whose progression halted and reversed which was restored to normal functioning by insulin alone or combined with insulin and other known therapeutic agents to treat age related macular degeneration.

Insulin exerts the trophic augmentation-amplification effects on the cell physiology without discriminating whether it is normal, metaplasic, dysplasic, heteroplasic, or carcinogenic (Philpott M P, Sanders D A, Kealey T. Effects of insulin and insulin-like growth factors on cultured human hair follicles: IGF-I at physiologic. J Invest Dermatol 1994; 102: 857-61, Shantha IBID). This is a known physiological phenomenon that the insulin does bind to the receptor sites of the IGF-I and insulin. The insulin exerts multiple profound physiological and pharmacological therapeutic effects. The insulin induces cell growth, (besides glucose transport) enhances the metabolism, and increases the glutathione needed for the cells' health.

This enhances mitosis and increases the production of nuclear proteins in the nucleus and ribonucleoprotein production by the endoplasmic reticulum, activates the Golgi complex, and enhances the lysosomes activity. Thus, the insulin helps to break up endocytosed materials and cellular debris to eliminate the cellular toxins which the insulin enhances (augmentation/amplification effects). The therapeutic effects of other pharmacological agents are reported (Shantha T. R., Life Extension September 2007: pp 74-79,) where insulin binds on the cell. This has been reported in the above publications. Thus, any dysfunction of the retina seen in age related macular degeneration will be restored back to normal using the described inventive methods. The present eye drops for the age related macular degeneration don't contain therapeutic agents to repair and to restore the damaged or disease afflicted rods. The tissues involved where the body uses its own physiological hormone locally as described in our invention.

Insulin, potassium, and glucose are routinely administered to treat low potassium levels in the cells even to this day. The inventor has used this method to lower the potassium levels in the blood for more than 3 decades. Insulin and glucose facilitates the entry of potassium inside the cell—a life saving measure. Similarly, the Insulin deposited in the conjunctival sac will enhance the uptake of therapeutic, pharmaceutical, biochemical, and biological agents or compounds by the dysfunctional cells of the retina, reduces the ROS to prevent further damage to the rods (cones) and to restore the function of the retina described in this inventive method.

The inventors have used insulin as potentiator of uptake and enhancer of therapeutic action of diverse therapeutic agents to cure and/or curtail curable acute, chronic, and incurable diseases such as cancer, Lyme disease, scleroderma, lupus, psoriasis, antibiotic resistant staphylococcus infection (MRSA infection), chronic wounds, neurological diseases, inner and middle ear affliction, autoimmune diseases, leprosy, prostate pathologies, skin diseases, herpes zoster of the eye with antiviral agents and tuberculosis.

Many other diseases have had good results with the method. The inventors have used insulin with other specific treatment modalities against depression, Alzheimer's, senile memory decline, Autism, Parkinson's, and many other neurological diseases successfully. The insulin needs to be delivered to the brain through proper routes where the routes of delivery to the CNS are going to be reported in later publications which we described in our utility patent application and on the rabies cure presentations (Shantha, T. R. Site Of Entry Of Rabies Virus Form The Nose And Oral Cavity; And New Method Of Treatment Using Olfactory Mucosa And By Breaking BBB, presented at The 2^{nd} International Rabies In Asia Conference Held In Hanoi, 2009, Pp 70-73, and The Rabies in the North Americus (XX RITA), held in Quebec City, 2009, Pp 20-21, Rabies cure, patent pending 2009).

The present inventors have used insulin for more than a decade to enhance the effectiveness of locally injected therapeutic agents, especially, cancers with chemotherapeutic agents with remarkable results. Our data supports that the insulin sprayed on indolent ulcers anywhere in the body, including the oral (gums), and the nasal cavity augmented the healing. Insulin stimulated the fibroblast, endothelial cell, angiogenesis, and skin cell growth resulting in accelerated wound healing.

Application of insulin soaked cotton swabs (1-3 units in normal saline) after teeth extraction induces rapid healing with reduced pain. Studies show that the application of insulin and antibiotics locally on the gums eliminated gum diseases (periodontitis), made the loose teeth firm, cleared the root infection rapidly with dental practices which the dental practices are under study (Dr. Hughes, J. DDS: Personal communication).

Insulin is a metabolic activity enhancer of all cells and therapeutic agents. Insulin can play an important role in treatment of many diseases including age related macular degeneration by increasing the metabolic activity, protecting against ROS damage, and preventing further degeneration of rod and cone segments (Shantha T. R.; 1. discovery of insulin and IPT: amazing history, 2. high dose methotrexate therapy using Insulin; 3. local injections of tumors with insulin and cytotoxic drugs; 4. two and three cycle insulin Potentiation therapy: Presented at 2^{nd} international conference on Insulin Potentiation Therapy held at Cancun, Mexico, Jun. 28-Jul. 1, 2004).

A synergy between certain membranes and metabolic effects of insulin on cell molecular biology increases therapeutic efficacy of all anti age related macular degeneration therapeutic, pharmaceutical, biochemical, and biological agents or compounds which the insulin reduces doses of the drugs, enhancing their uptake with augmentation/amplification effects greater than before the therapeutic efficacy. The insulin enters the cells where the insulin increases the effectiveness of therapeutic agents many properties. Thus, the present inventive method not only enhances the uptake of therapeutic agents. The insulin enhances their therapeutic effect inside the cells of the disease afflicted cells as reported by Alabaster (IBID).

It is known that the pharmaceutically acceptable oxidizing agent facilitates the delivery of the bioactive agent through the skin and mucous membranes which the membranes includes the oral cavity, nasal passages, and conjunctiva. In general, the oxidizing agent can react with molecules present in the conjunctiva where a reaction of adversity with the bioactive agent. For example, the reduction of the glutathione which glutathione is present in the mucus membranes and the skin can inactivate bioactive agents such as insulin by breaking chemical molecular bonds.

Not wishing to be bound by theory, when delivering insulin through the skin and mucous membranes, reduced glutathione that it can inactivate insulin. Specifically, insulin has numerous disulfide bonds which are crucial for the protein conformation, biological activity, and subsequent therapeutic effects. Reduced glutathione will inactivate insulin by reducing or breaking insulin's disulfide bonds. Once these disulfide bonds are broken; the insulin becomes inactive due to lost protein conformation and biological activity. Thus, the administration of the oxidant by eye drops (as described by Shantha et al in U.S. Patent Application Pub. No. 2009/0347776 A1) herein, prevents the inactivation of the bioactive agent like insulin when applied to the skin, mucus membrane, and conjunctival sac of the eye.

Specifically, application of an oxidant or a pharmaceutically oxidizing agent to conjunctival sac will lower or prevent the effects of reduced proteins. The reduction of the biological molecules has on the bioactive agents which the inactivation of bioactive agents via reduction or cleavage of crucial molecular bonds will be avoided. The selection and the amount of the pharmaceutically acceptable oxidizing agent can vary depending upon the bioactive agent that agent is to be administered. In one aspect, the oxidizing agent includes, which is not limited to iodine, povidone-iodine, and any source of iodine or combinations of oxidants, silver protein, active oxygen, potassium permanganate, hydrogen peroxide, sulfonamides, dimethyl sulfoxide or any combination thereof. These oxidizing agents may act as absorption agents which the oxidizing agents help facilitate delivery of a therapeutic agent onto and into the skin. In one aspect, the oxidant is at least greater than 1% weight per volume, weight per weight, or mole percent.

Our preliminary studies have shown that the conjunctiva unlike normal skin and other mucus membranes don't act as a barrier like stratum corneum of the skin for entry of insulin due to the paucity of the presence of reduced glutathione. Our studies show that the conjunctiva doesn't contain any insulin blocking agent. The conjunctiva doesn't have the multilayered stratum coneium as seen on the skin which can block the therapeutic agents' entry from the skin.

The insulin deposited in the conjunctival sac is rapidly absorbed by the conjunctiva, cornea, and bulbar conjunctiva, retina, choroid, ciliary body and processes, iris, anterior and posterior chambers of the eye, retro bulbar space and helps the entire retina including the photoreceptors to recover from age related macular degeneration affliction and any pathological states affecting the vision. The insulin prevents the progression of age related macular degeneration.

In one aspect, transconjunctival penetration of insulin and therapeutic, pharmaceutical, biochemical and biological agents or compounds can be facilitated by enhancers. The enhancers can be used to further expedite the entry of these agents to penetrate and to permeate inside the eye ball where the agents are delivered to choroid and retina.

Penetration enhancers not only penetrate a membrane efficiently; these enhancers also enable other bioactive agents to cross a particular membrane or barrier more efficiently. Penetration enhancers produce their effect by various modalities such as disrupting the cellular layers of the conjunctival sac surface interacting with inter and intracellular proteins and lipids, or improving partitioning of bioactive agents as they come into contact with the mucosal membranes. The entry into BV and Lymphatics of the eye which the BV dissipates them to the contents of the eye ball within the retina.

These enhancers, macromolecules up to 10 kDA are able to pass through the conjunctival sac layers of the eyes where they reach the site of age related macular degeneration which the blood vessels, ARMD and retina are undergoing pathological changes. These enhancers should be non-toxic, pharmacologically inert, and non-allergic substances. In general, these enhancers may include anionic surfactants, urea's, fatty acids, fatty alcohols, teARMDenes, cationic surfactants, nonionic surfactants, zwitterionic surfactants, polyols, amides, lactam, acetone, alcohols, and sugars.

In one aspect, the 10 penetration enhancer includes dialkyl sulfoxides like dimethyl sulfoxide (DMSO), decyl methyl sulfoxide, dodecyl dimethyl phosphine oxide, octyl methyl sulfoxide, nonyl methyl sulfoxide, undecyl methyl sulfoxide, sodium dodecyl sulfate and phenyl piperazine, or any combination thereof.

In another aspect, the penetration enhancer may include lauryl alcohol, diisopropyl sebacate, oleyl alcohol, diethyl sebacate, dioctyl sebacate, dioctyl azelate, hexyl laurate, ethyl caprate, butyl stearate, dibutyl sebacate, dioctyl adipate, propylene glycol dipelargonate, ethyl laurate, butyl laurate, ethyl myristate, butyl myristate, isopropyl palmitate, isopropyl isostearate, 2-ethylhexyl pelargonate, butyl benzoate, benzyl benzoate, benzyl salicylate, dibutyl phthalate, or any combination thereof which are opthalmologically acceptable to be used for local instillation.

In other aspects, these additional components with insulin may include antiseptics, antibiotics, anti-virals, anti-fungals, anti-inflammatories, anti-dolorosa, antihistamines, steroids, vasodilators and/or vasoconstrictors to reduce inflammation, irritation, or reduce rapid absorption through conjunctival sac. Such vasoconstrictors may include phenylephrine, ephedrine sulfate, epinephrine, naphazoline, neosynephrine, vasoxyl, oxyrnetazoline, or any combinations thereof.

Such anti-inflammatories may include non-steroidal anti-inflammatory drugs (NSAIDs). NSAIDs alleviate pain and inflammation by counteracting Cyclooxygenase and preventing the synthesis of prostaglandins. In one aspect, NSAIDs include celecoxib, meloxicam, nabumetone, piroxicam, napmxen, oxaprozin, rofecoxib, sulindac, ketoprofen, valdewxid, anti-tumor necrosis factors, 10 anti-cytokines, anti-inflammatory pain causing bradykinins or any combination, thereof.

Such antiseptics, anti-virals, anti-fungals, and antibiotics, may include ethanol, propanol, isopropanol, or any combination thereof. Quaternary ammonium compounds includes which is not limited to benzalkonium chloride, cetyltrimethylammonium bromide, cetylpyridinium chloride, benzethonium chloride, or any combination thereof: boric acid, chlorhexidine gluconate, hydrogen peroxide, iodine, mercurochrome, ocetnidine dihydrochloride, sodium chloride, sodium hypochlorite, silver nitrate, colloidal silver, mupirocin, erthromycin, clindamycin, gentamicin, polymyxin, bacitracin, silver, sulfadiazine, or any combination thereof.

The present invention uses insulin with the above described anti-inflammatory and antibacterial agents. These can eliminate the pathogenic factors contributing to the age related macular degeneration and to restore normal sight.

In accordance with one aspect of the invention, the compounds applied locally to the eye's site are mixed conjunctivally which the conjunctiva is a suited vehicle or carrier. The compositions of this invention may comprise aqueous solutions such as e.g., physiological saline, oil, gels, patches, solutions or ointments. The vehicles which carry these biologically active therapeutic agents may contain conjunctivally compatible preservatives such as e.g., benzalkonium chloride, surfactants like e.g., polysorbate 80, liposome's or polymers: examples like methyl cellulose, polyvinyl alcohol, polyvinyl pyrrolidone, and hyaluronic acid and others. Sterile water or normal saline are used in some of the preparations of the eye drops for our invention.

There are various forms of insulin used to treat diabetes which different forms of insulin can be formulated to be used in this invention. They are grouped under rapid, short, intermediate, and long acting insulin. The insulin is dispensed as premixed form containing rapid to long acting insulin. Insulin products are categorized according to their putative action (see Table IV) profiles as:

Rapid-acting: insulin lispro, insulin aspart, and insulin glulisine

Short-acting: regular (soluble) insulin

Intermediate-acting: NPH (isophane) insulin

Long-acting: insulin glargine and insulin detemir

The table I summarizes: the time of onset; peak action and duration of action of the different types and the different brands of insulin that the insulin can be used in our invention.

TABLE 1

| | | | | |
|---|---|---|---|---|
| Insulin Preparation | Onset of | Peak | Effective | Maximum |
| and their generic | Action in | action in | duration of | duration in |
| and trade names | hours | hours (h) | action (h) | hours |

RAPID - ACTING INSULIN ANALOGUES AND PREPARATIONS

| | | | | |
|---|---|---|---|---|
| Insulin lispro | ¼-½ | ½-1¼ | 3-4 | 4-6 |
| (Humalog), Insulin | | | | |
| aspart (NovoLog), | | | | |
| Insulin glulisine | | | | |
| (Apidra) | | | | |

SHORT - ACTING INSULIN

| | | | | |
|---|---|---|---|---|
| Regular (soluble) | ½-1 | 2-3 | 3-6 | 6-8 |

INTERMEDIATE-ACTING

| | | | | |
|---|---|---|---|---|
| NPH (isophane) | 2-4 | 6-10 | 10-16 | 14-18 |

LONG - ACTING INSULIN ANALOGUES

| | | | | |
|---|---|---|---|---|
| Insulin glargine | 3-4 | 8-16 | 18-20 | 20-24 |
| (Lantus) | | | | |
| Insulin detemir | 3-4 | 6-8 | 14 | ~20 |
| (Levemir) | | | | |

Glucose concentrations are expressed as milligrams per deciliter (mg/dL or mg/100 mL) in the United States, Japan, Spain, France, Belgium, Egypt, and Colombia. The millimoles per liter (mmol/L or mM) are the units used in the rest of the world. Glucose concentrations expressed as mg/dL can be converted to mmol/L by dividing by 18.0 g/dmol (the molar mass of glucose). For example, a glucose concentration of 90 mg/dL is 5.0 mmol/L or 5.0 mM.

During a 24 hour period blood plasma glucose levels are typically between 4-8 mmol/L (72 and 144 mg/dL). Although, 3.3 or 3.9 mmol/L (60 or 70 mg/dL) is referred to as the lower limit of normal glucose. The symptoms of hypoglycemia typically do not occur until 2.8 to 3.0 mmol/L (50 to 54 mg/dl) glucose levels are reached. The precise level of glucose considered low enough to define hypoglycemia is dependent on (1) the measurement method, (2) the age of the person, (3) presence or absence of effects (symptoms), and (4) the purpose of the definition. The debate continues to what degree of hypoglycemia warrants medical evaluation or treatment, or can cause harm.

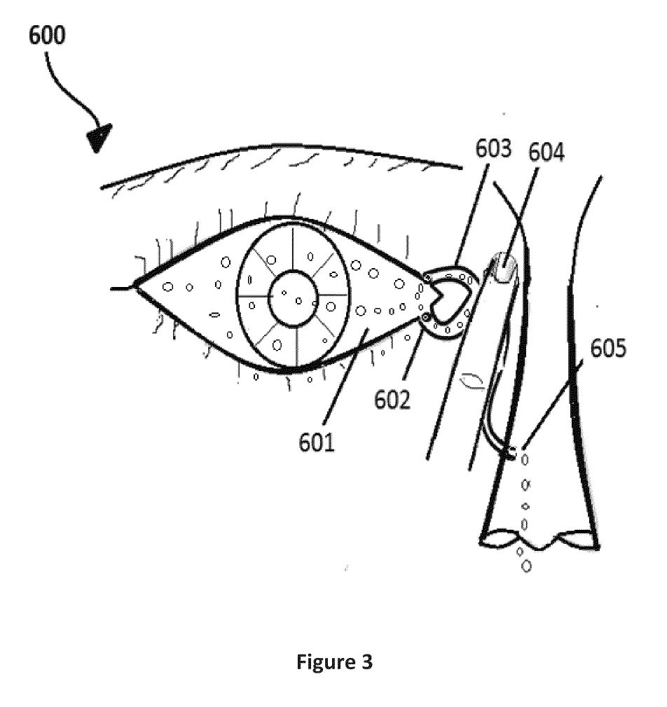

One has to realize the possibility of developing hypoglycemia when the insulin is being used as ophthalmic drops due to nasal mucosal absorption draining through the nasolacrimal ducts (FIG. 3). Patients will be warned about the possibility of hypoglycemia which they will be prepared for a hypoglycemic reaction. FIG. 3 shows the prevention of the drainage to the nose.

Generally, the hypoglycemia is defined as a serum glucose level (the amount of sugar or glucose in a person's blood) below 70 mg/dL. Symptoms of hypoglycemia, in general, appear at levels below 60 mg/dL. Some people may experience symptoms above this level. Levels below 50 mg/dL affect the brain function. Signs and symptoms of hypoglycemia include erratic or rapid heartbeat, sweating, dizziness, confusion, unexplained fatigue, shakiness, hunger, feeling hot, difficulty in thinking, and headache. Some may be even develop seizures and potential loss of consciousness with severe hypoglycemia.

Once symptoms of hypoglycemia develop, the patient should be treated with oral ingestion of a fast-acting carbohydrate such as glucose tablets, fruit juice, fruit bowl, chocolate bar, or regular Coca-Cola, sugary drinks or eat plain sugar followed with a drink of water or IV administration of 25% glucose if there is severe hypoglycemic which the patient has an IV established. It is important to test the blood sugar 15 minutes after administration if symptoms of hypoglycemia develop with a finger stick sugar tester strips. It has been projected that the newborn brains are able to use alternate fuels when glucose levels are low more readily than adults.

Preparation of the Age Related Macular Degeneration Patients for Therapy Using Our Inventive Method of Using Insulin

Before using described inventive methods and examples; a thorough examination of the affected patient's eye is in order. The examination of the eye may include: 1. Acuity testing, 2. Biomicroscopy, 3. intraocular pressure (10P), 4. Ophthalmoscopy, 5. Color vision test, 6. Tear osmolality, 7. Schimer's test, 8. Tear film breakup time (tBUT), 9. Test for Superficial punctate keratitis (SPK), 10. Fluorescein and Rose Bengal staining (RBS) of BV of the retina, as well as cornea, conjunctiva, and eyelids, 11, slit-lamp examination of the conjunctiva, cornea, anterior chamber, iris, and lens, 12. The Ocular Surface Disease Index (OSDI), 13. Microscopic examination of the tear filament, 14. Maturation index (a Papanicolaous stained sample of conjunctival epithelium). 15. Electroretinogram (ERG) to measure the function of the photoreceptors if age related macular degeneration is suspected.

In addition, a complete physical examination with blood test for thyroid, parathyroid, growth hormone, insulin, IGF-1, FSH, LH, cortisol, estradiol, and testosterone levels, electrolytes, blood cell count, cholesterol levels, ESR, and a urine sample for pregnancy test when this is deemed necessary when the patient is of childbearing age.

To apply our inventive ophthalmic insulin drops as therapeutic agents, the patient or the care giver has to wash their hands with a mild antiseptic soap. The person or patient applying the drops must be careful not to touch the dropper tip to the eye lids (and the foreign objects) to avoid contamination if there is an eye lid infection. Tilt the head back, or lay down with head extended on a neck pillow, gaze upward and backwards, and pull down the lower eyelid to expose the conjunctival fornix. Place the dropper directly over the eye away from the cornea and instill the prescribed number of drops. Look downward and gently close your eyes for 1 to 2 minutes. The patient should not rub the eye. Do not rinse the dropper unless the patient or person knows the sterilization technique with hot water. If other therapeutic, pharmaceutical, biochemical and biological agents or compounds are to be selected to treat the condition with our invention. The patient should wait at least 3-5 minutes before using other selected anti-age related macular degeneration therapeutic agents or the other variety of ophthalmic medicaments. It is important to instill medications regularly as prescribed to control age related macular degeneration. Consult your doctor and/or pharmacist if the systemic medications that you are taking are safe to use with the eye drops described and prescribed. When there is no contraindication for the insulin eye drops, you can treat patients, except, the patients with hypoglycemia syndromes and in some cases external ocular tumors.

To minimize the absorption into the bloodstream and to maximize, the amount of drug absorbed by the eye, close your eye for one to five minutes after administering the insulin drops. Then, press your index finger gently against the inferior nasal corner of your eyelid to close the tear duct which drains into the nose (FIG. 3). This will prevent any adverse systemic effects due to nasal vascular uptake into the systemic circulation from the nasolacrimal duct drainage of the therapeutic agents from the conjunctival sac.

Eye drops may cause a mild uncomfortable burning or light stinging sensation which this reaction should last for only a few seconds. The anti-age related macular degeneration drops take effect after 5-10 minutes after application depending upon the therapeutic agents used with the eye drops. We recommend that it is best to use insulin eye drops before bed time and rising in the morning. This process can be repeated every 6, 12 or 24 hours for 3-7 days a week till the desirable results are obtained. Age related macular degeneration patients can use insulin eye drops all their lives or intermittently, depending on the results and the need. The therapeutic agents are instilled using a sterile dropper (or bottle with medication equipped with a dropper nipple) into the conjunctival sac.

Experiments by the present inventors has shown that the local application of rapid acting or other types of insulin formulations on the balding scalp, eye lid hair line, on the gums, oral and nasal mucosa, and conjunctival sac, surgical wounds, open area of extracted wisdom teeth, local injections of tumors, injection into tunica vaginalis testes, other regional and local sites did not change the blood sugar levels (without hypoglycemic effects) indicates, that there is safety to use up to 1, 2 or 3 IU (international units) insulin to the conjunctival sac of both eyes without hypoglycemia effects. The present invention formulations contain only 0.10 to 1.00 IU per drop which the dosage can be increased or the dosage can be decreased depending upon the disease states.

Preparation of Insulin Eye Drops for Use in Age Related Macular Degeneration

Take 100 international units (IU) of rapid or intermediate or long acting insulin (or IGF-1) and dilute in 5 ml of sterile saline or distilled water or other carriers and facilitators as described above. The pH can be adjusted to prevent the sting when the insulin is dropped into the conjunctival sac. The preparation can contain nanograms (micrograms) of local anesthetics to prevent the stinging when the eye drops are applied to the eye. In this preparation, each ml contains 20 units of insulin.

In pharmacies, a drop was another name for a minim, which a drop would be 0.0616 milliliters. The drop is standardized in the metric system to equal exactly 0.05 milliliters. The 20 drops equal one ml (1 cc) which each drop contains 0.10 IU of insulin. The concentration of the insulin content can be increased to 0.20, 0.30, 0.40, and 0.50 IU or even up to 1 or 2 or 3 unit of insulin. The insulin content can be increased per drop in the dilutant preparation. The insulin content can be decreased by reducing the insulin units used for the preparation of the ophthalmic drops. Instill one to two drops to each eye lower lid fornix and/or everted upper eyelid (conjunctival sac) as a single agent. The applicant must apply pressure on the nasolacrimal duct as shown in the FIG. 3 to prevent drainage into the nasal cavity.

If other combinations of the anti-age related macular degeneration therapeutic agents are to be used: first use insulin drops, wait for 3-5-10 minutes and apply the other therapeutic, pharmaceutical, biochemical, and biological agents or compounds. After this procedure, instill one more insulin drop to further enhance the uptake of the other selected therapeutic agents to augment-amplify their effects at the cellular level.

This step is optional and may not be needed in most cases. The dose used in our invention is appropriately selected depending upon symptom, age, and severity of the disease, dosage form, and existing health conditions. The pH can be within a range which the pH is acceptable to ophthalmic preparations which the pH preferably is within a range from 4-6-7 to 8 most preferably 7.4.

The data supports the other therapeutic agents which the agents are used after insulin where the agents are prepared in 5-10% solutions of glucose. The glucose acts as a carrier of the therapeutic agents after pretreatment with insulin. I have named this Porcesses as “local Insulin Potentiation Therapy (LIPT)”.

Insulin can be compounded as a liquid ophthalmic isotonic solution containing cyclosporin, or other antiautoimmune therapy agents, or vitamins, and one or more one buffering agents, said buffering agents producing a pH in said composition similar to mammalian eye fluids.

The insulin pharmaceutical eye drop preparation of this invention may contain 0.25%-0.5%-1%-2% or more glucose. There are several mechanisms which glucose and insulin protect the damaged cells that the insulin restores normal function. Glucose is the preferred substrate during periods of cell damage and ischemia. Adenosine triphosphate derived from glycolysis is vital for stabilization of membrane ion transport which electroporation, iontophoresis, sonophoresis, vibroacoustic and vibration methods transport can enhance.

The biological activity is enhanced by insulin. This is crucial to the above biological activity needed for cellular integrity, endothelium, vascular smooth muscle cells, and nerve cells like the retina, photoreceptors and their synapses. Preservation of these functions in these structures of the eye, especially, the retina decreases any further damage and participates in the repair. Glucose esterifies intracellular free fatty acids, which these decreases their toxic end-products and oxygen free radicals.

Glucose is a direct precursor of pyruvate, which pyruvate is carboxylated to the citric acid cycle substrates malate and oxaloacetate which this can replenish depleted substrates, thus, stimulating oxidative aerobic metabolism, reduce the ROS production and their adverse effect on photoreceptors. Glucose with the help of insulin esterifies intracellular free fatty acids which the fatty acids decreases their toxic end-products and oxygen free radicals.

Experimental studies have shown that glucose converted to pyruvate with the help of insulin can restore the function through the replenishment of depleted citric acid substrates. This helps in the repair and the restoration of the photoreceptors cellular function. This helps in curtailing or in curing the age related macular degeneration. Experimental studies have shown that the glucose is converted to pyruvate in the presence of insulin which the insulin can restore contractile function of the blood vessel, various histological components of the retina, choroid and ciliary muscles through the replenishment of depleted citric acid. Thus, our invention with the use of insulin with glucose can help in relieving and reversing the age related macular degeneration pathology, signs, symptoms, and restore the physiological state to the pigment epithelial cells.

Insulin stimulates pyruvate dehydrogenase activity, which the activity in turn stimulates aerobic metabolism. Exogenous insulin helps to reverse insulin resistance which this reversal can be of benefit in age related macular degeneration associated with diabetes. The importance is the glucose which the insulin facilitates the entry of therapeutic, pharmaceutical, biochemical, nurticeuticals, biological agents or compounds, and drugs into the normal and disease afflicted cells in the eyes and other parts of the body.

The above pharmaceutical eye drop preparation of our invention may contain antibacterial components which these components are non-injurious to the eye when used. Examples are: thimerosal, benzalkonium chloride, methyl and propyl paraben, benzyldodecinium bromide, benzyl alcohol, or phenyl ethanol. There is an autism controversy which we will avoid using thimerosal.

The therapeutic pharmaceutical preparation may contain buffering ingredients such as sodium chloride, sodium acetate, gluconate buffers, phosphates, bicarbonate, citrate, borate, ACES, BES, BICINE, BIS-Tris, BIS-Tris Propane, HEPES, HEPPS, imidazole, MES, MOPS, PIPES, TAPS, TES, and Tricine.

The therapeutic, pharmaceutical, biochemical, and biological agents or compounds used in our invention may also contain a non-noxious pharmaceutical carrier, or with a non-toxic pharmaceutical inorganic substance. Typical of pharmaceutically acceptable carriers are, for example: water, mixtures of water and water-miscible solvents such as lower alkanols or aralkanols, vegetable oils, peanut oil, polyalkylene glycols, petroleum based jelly, ethyl cellulose, ethyl oleate, carboxymethyl-cellulose, olyvinylpyrrolidone, isopropyl myristate and other traditionally acceptable carriers.

The therapeutic preparation may contain non-toxic emulsifying, preserving, wetting agents, and bodying agents. For example: polyethylene glycols 200, 300, 400 and 600, carbowaxes 1,000, 1,500, 4,000, 6,000 and 10,000, antibacterial components as quaternary ammonium compounds, methyl and propyl paraben, benzyl alcohol, phenyl ethanol, buffering ingredients such as sodium borate, sodium acetates, gluconate buffers, and other conventional ingredients such as sorbitan monolaurate, triethanolamine, oleate, polyoxyethylene sorbitan monopalmitylate, dioctyl sodium sulfosuccinate, monothioglycerol, thiosorbitol, ethylenediamine tetracetic. Furthermore, appropriate ophthalmic vehicles can be used as carrier media for the current purpose. This includes conventional phosphate buffer vehicle systems which are isotonic boric acid vehicles, isotonic sodium chloride vehicles, isotonic sodium borate vehicles and the like.

The objects are accomplished by treating the eye with an aqueous composition containing an effective amount of a nonionic surfactant and insulin. The applicant has found that an effective amount of surfactant may comprise anywhere from 0.5 percent by weight and by volume to about 10 percent by weight and volume (hereinafter %), preferably about 1-5%, of active surfactant (not combined with oil) in the composition combined with insulin. However, the use of any oil in the composition will reduce the effectiveness of the surfactant.

The reason is that a substantial percentage of the surfactant tends to serve as a vehicle for dissolving or forming an emulsion of the oil with the aqueous layer to “wash” or hydrate the corneal surface. Thus, any oil is used in the composition, then, additional surfactant will be required to provide the effective amount of 0.5-10% preferably 1-5% of available active nonionic surfactant.

The anti-age related macular degeneration therapeutic agents' preparation may contain surfactants such as polysorbate surfactants, polyoxyethylene surfactants (BASF Cremaphor), phosphonates, saponins, and polyethoxylated castor oils. The preference is the polyethoxylated castor oils which are commercially available.

The pharmaceutical preparation may contain wetting agents which the agents are already in use in ophthalmic solutions such as carboxy methyvl cellulose, hydroxypropyl methylcellulose, glycerin, mannitol, polyvinyl alcohol or hydroxyethylcellulose. The diluting agent may be water, distilled water, sterile water, or artificial tears. The wetting agent is present in an amount of about 0.001% to about 10%.

The ophthalmic formulation of this invention may include acids and bases to adjust the pH, tonicity imparting agents such as sorbitol, glycerin and dextrose, other viscosity imparting agents such as sodium carboxymethylcellulose, polyvinylpyrrdidone, polyvinyl alcohol, and other gums. The suitable absorption enhancers are surfactants, bile acids. The stabilizing agents are antioxidants, like bisulfites and ascorbate. The metal chelating agents like sodium EDTA and drug solubility enhancers which are the polyethylene glycols. These additional ingredients help give commercial solutions stability which they don't need to be compounded.

Ophthalmic medications compositions will be formulated to be compatible with the eye and/or contact lenses. The eye drop preparation should be isotonic with blood. The ophthalmic compositions, which are intended for direct application to the eye, will be formulated to have a pH and tonicity which these are compatible with the eye. This will normally require a buffer to maintain the pH of the composition at or near physiologic pH (i.e., pH 7.4) which the buffer may require a tonicity agent to bring the osmolality of the composition to a level or near 210-320 millimoles per kilogram.

In the following detailed, description of the invention, reference is made to the drawings, microphotographs and tables which reference numerals refers to the like elements which the elements are intended to show by way of illustration specific embodiments where the invention we describe using insulin, and IGF-1 with or without other known anti age related macular degeneration therapeutic, pharmaceutical, biochemical, and biological agents or compounds enumerated, They may be prescribed and practiced. This is understood where other embodiments may be utilized that the structural changes may be made without departing from the scope and the spirit of the invention described herein.

The eye drop composition of the invention includes buffering agents to adjust the acidity or the alkalinity of the final preparation to prevent eye irritation. The composition is an isotonic solution in that it has the similar pH to fluids indicating that the pH of the composition is 6.1, 6.3, or 7.4. The buffering agents may include all of zinc sulfate, boric acid, and potassium necessary to be effective in achieving the pH of the composition of from 6.10 to 6.30, and to 8.00 typically. The total amount of buffering agents present in the composition ranges from 1% to 10% by weight of the composition.

The eye drop composition includes a lubricant such as cellulose derivatives (carboxymethyl cellulose). The composition may contain known preservatives conventionally used in eye drops such as benzalkonium chloride and other quaternary ammonium preservative agents, phenyl mercuric salts, sorbic acid, chlorobutanol, disodium edentate (EDTA), thimerosal, methyl and propyl paraben, benzyl alcohol, and phenyl ethanol. Purified benzyl alcohol may be in the concentration preferably from 0.1% to 5% by weight.

The eye treatment composition of the invention is a solution having a vehicle of water or mixtures of water and water-miscible solvents. For example, lower alkanols or arylaikanols, the phosphate buffers vehicle systems and isotonic vehicles where the vehicles are boric acid, sodium chloride, sodium citrate, sodium acetate and the like, vegetable oils, polyalkylene glycols, and petroleum based jelly, as well as aqueous solutions containing ethyl cellulose, carboxymethyl cellulose, and derivatives thereof. The hydroxypropylmethyl cellulose, hydroxyethyl cellulose, carbopol, polyvinyl alcohol, polyvinyl pyrrolidone, isopropyl myristate, and other conventionally-employed non-toxic, pharmaceutically acceptable organic and inorganic carriers.

The composition is applied to the eye should be sterile in the form of an isotonic solution. The constitution may contain non-toxic supplementary substances such as emulsifying agents, wetting agents, bodying agents, and the like. For example, polyethylene glycols, carbowaxes, and polysorbate 80 and other conventional ingredients can be employed such as sorbitan monolaurate, triethanolamine, oleate, polyoxyethylene sorbitan 35 monopalmitylate, dioctyl sodium sulfosuccinate, monothioglycerol, thiosorbitol, ethylenediamine tetraacetic acid, and the like.

Maintenance of Photoreceptors, Muller Cells, Retinal Pigment Epithelium and Choroidal Blood Vessel Function by Insulin Ophthalmic Therapeutic Agent of Our Invention

A wide array of blinding and visually impairing disorders including age related macular degeneration are caused by degeneration of the photoreceptors of the retina. The retina is a intricate stratum structure comprising 10 layers of neuronal cell types, their synapses, and their axons, as well as the complex Muller glial cells, and their arrangement on retinal pigment epithelium (RPE). The health and the continued existence of the photoreceptors are greatly dependent on the integrity of other surrounding cell types of the retina, especially RPE cells, and the Muller cells. RPE-secrete proteins including pigment epithelium-derived factor (PEDF) to promote photoreceptor differentiation and survival of the photoreceptor.

Our invention with the use of insulin will augment the production of PEDF from the RPE cells to maintain the photoreceptors cells integrity and their physiological state. PEDF may in fact act as antiangiogenic, which prevents the formation of neovascularization of the choriocapillaries and their invasion towards foveal photoreceptors cells as seen in ARMD.

The Muller cells of the retina are recognized to play important roles in photoreceptor development and survival. Muller cells are coupled embryologically, physically, and metabolically to photoreceptors. The Muller cells give and bestow trophic support to promote photoreceptor survival which the survival may regulate synaptogenesis and neuronal processing through bidirectional communication. Delivery of insulin in ophthalmic drops will help to maintain the integrity of Muller cells. This helps to maintain the structure and the function of the photoreceptors which the Muller cells play a role in the treatment of age related macular degeneration.

Even now, the mechanisms of how the numerous genetic mutations or other changes in the photoreceptors of ARMD patients could give rise to damaging free-radical reactions (ROS) capable of triggering apoptosis through their adverse effects on RPE, mitochondria's and photoreceptors outer segment function isn't known at this time. One of the significant parts of our invention is to focus on free radical adverse effect of ROS reactions in ARMD where the invention will provide a rationale simple therapy by use of wide-ranging array of antioxidants and nutritional supplements with insulin for stemming progression of ARM D.

In particular, our invention focuses on saving photoreceptors not affected by the genetic problems of the cones and rods, which the cells can become lethally damaged by a spill-over of free radicals and related harmful chemical reactions occurring in the rods, RPE and neochoriocapillares. Photoreceptors, amacrine and horizontal cells of the retina undergo neurite sprouting in human retinas with age related macular degeneration. These changes in the retinal neurons may contribute to the electroretinographic abnormalities and the progressive decline in vision noted by patients with age related macular degeneration.

Photoreceptors are structurally polarized neurons with one pole of the neurons that are the chemical synapses. The other end is the outer segment which is the most highly specialized region of the photoreceptor cells where the vision originates. Our invention of using insulin will help to maintain the integrity of the choroid, Bruch's membrane, retinal pigment epithelium, Muller cells, and the most sensitive parts of photoreceptors (the outer segment with the mitochondria) by providing needed metabolic, nutritional trophic factor support, and by facilitating the removal of the ROS from the site and supporting physiological functioning of these structural units.

This and other metabolic and therapeutic qualities of the insulin will prevent the development, stop the progression, and curtail or cure the age related macular degeneration. I have used insulin ophthalmic drops for various oculopathies, including, age related macular degeneration for years with great success.

Free Radical Damage in Age Related Macular Degeneration: Our Inventions to Prevent, Curtail or Cure Free Radical Damage which are Involved in Age Related Macular Degeneration Development

The pathophysiology of the age related macular degeneration isn't known. ARMD is the result of a defect in the physiological mechanisms of the protection against the photo-oxidative processes involving free radicals (ROS) due to pathology involved in angiogenesis and destruction of RPE. The pathological processes in ARMD involve the choriocapillaries, Bruch's membrane, RPE, and ultimately the victim's photoreceptors of the Macula. The retinal degeneration is the result of a deficiency in the protective physiological mechanisms from the RPE and relentless attack by the sprouting choriocapillaries. They literally destroy everything on their way as they continue to sprout and grow towards the photoreceptors.

The objective of the discovery of the drug to treat ARMD should encompass: 1. Protection against the photo-oxidative processes involving free radicals (ROS), 2. Attenuate and ease the biological effects of sun radiations on the retina during vision perception by the retinal cones and rods, 3. Maintain the proper physiological milieu for the photoreceptors and their organelle to function; at the same time arrest any evolving pathological conditions, 4. Prevent the destruction of the RPE, 5. Maintain the integrity of the Bruch's membrane, 6. Arrest or slowdown the choroidal neovascularization. Our invention does fulfill these objectives and more.

The body is made up of many diverse types of cells composed of different types of molecules. Molecules are made up of one or more atoms bound to each other forming a molecule—i.e. one or more elements joined by chemical bonds. The atoms consist of a nucleus, a mix of positively charged protons, electrically neutral neutrons, and the central nucleus surrounded by a cloud of negatively charged electrons bound to nucleus by electromagnetic force. The number of protons (positively charged particles) in the atom's nucleus determines the number of electrons (negatively charged particles) surrounding the atom. Electrons are involved in chemical reactions which the electrons are the substance that bonds atoms together to form molecules.

Electrons surround, or “orbit” an atom in one or more shells. The innermost shell is full when it has two electrons. When the first shell is full, electrons fill the second shell. When the second shell has eight electrons, the shell is full, and the process continues. Free radicals are oxygen atoms. The oxygen atoms are missing one electron from the pair which the atoms are endowed naturally. When an atom is missing an electron from a pair, the atom becomes unstable and reactive which the atom wants to find another electron (ROS) to fill in the missing electron in the gap. Hence, the atom grabs an electron from the next atom. When the atom is near, a free radical seizes an electron from another atom, the second atom becomes a free radical which this process starts a cascade of new free radicals in our body like the atomic chain reaction. Once the process is started, the process can continue which the process results in the disruption of a living cell function leading to disease states of many kinds from age related macular degeneration to cancers.

There are numerous types of free radicals formed within the body. We focus on the oxygen-centered free radicals or ROS because the retina and the photoreceptors are very sensitive to oxygen which affects the free radicals. The majority of common ROS incorporate: 1. the superoxide anion (O2-), 2 the hydroxyl radical (OH.), 3 singlet oxygen (1O2), and 3 hydrogen peroxide (H2O2) Superoxide anions are formed when oxygen (O2) acquires an additional electron, which the molecule is the only one unpaired electron. For example, the hydrogen peroxide is produced where the H₂O₂ can be converted to the highly damaging hydroxyl radical or be catalyzed or excreted harmlessly as water.

Glutathione peroxidase is essential for the conversion of glutathione to oxidized glutathione which H₂O₂ is converted to water. If H₂O₂ is not converted into water, one O₂, singlet oxygen is formed which is not a free radical. The singlet oxygen can act as a catalyst for the free radical formation. The molecule can interact with other molecules leading to the formation of a new free radical. Zinc is one of the most important metals, which zinc exists in one valence (Zn2+) which the Zinc does not catalyze free radical formation.

Age related macular degeneration results due to damage by ROS, besides other etiological factors. This is substantiated by delay in progression of the disease by the use of Vitamin A, E, and C which the vitamins are important known antioxidants. Zinc, unlike, other metals acts to stop free radical formation by displacing those metals which the metals do have more than one valence including iron. Every time the light comes in contact with the photoreceptors and RPE, the mitochondria O2-is endlessly being formed. Our invention of using insulin and other therapeutic agents reduces these ROS, prevent the photoreceptors damage, and augment the protection of the photoreceptors, which this process prevents further damage where the progression of age related macular degeneration is delayed or halted.

What do the free radicals' do once they are formed? The free radicals stagger, stumble, splash around, and seize electrons from adjacent cells—which the free radicals do an assortment of damage to them at the same time. The ultraviolet light in sunshine (skin cancer and cataracts); Toxins of all sort includes the following: tobacco smoke, the chemicals found in our food with lack of antioxidants, the poisonous wastes of our bodies own metabolism, and man-made toxins like air pollution, drugs, and pesticides are some of the culprits.

More or less, every cell in our body comes under attack from a free radical once every ten seconds which the cell attack is blamed for cancers, heart diseases, age related macular degeneration, neurodegenerative diseases, and a host of other diseases. Sometimes the body's immune systems' cells purposefully create free radicals to neutralize viruses and bacteria as seen in WBE and immune system.

The photosensitive cells of the retina and RBE in essence avascular are easily subject to free radicle damage due to light hitting the receptors continuously for almost 16 hours a day. The photoreceptors, are genetically defective, the production of ROS, and the effect of ROS is amplified when the results are in their dysfunction and damage, ultimately.

Apoptosis contributes to the age related macular degeneration of the eyes with segmental or total loss of vision. The light from the sun or other sources will generate free radicals which the radicals can cause more damage. The free radicals accelerate the age related macular degeneration development that ARMD leads to blindness if there is no innate (inherent) defense against ROS.

In age related macular degeneration the defense against ROS is inhibited, lacking, or missing. Our invention of use of insulin with antioxidants such as Vitamin A, E, C, GLA, Omega 3, and Glutathione and other natural supplements can be of immense therapeutic value in treating this condition. Normally, the body can handle free radicals if antioxidants are unavailable. If the free-radical production becomes excessive in age related macular degeneration due to constant bombardment of light on the photoreceptors; the results will be damage to the retina, in particular sensitive photoreceptors.

Free radicals are present in all living cells. Free radicals are a part of the cell metabolic life processes. Free radicals have an incredibly short half-life; hence, the free radicals are not easy to measure in the laboratory which the short half life of the free radical increases the expense to study and to test. However, excessive free radicals in our cells can attack the cell membranes (the outer coat of the cell and delicate folded lamellae of rods and cones outer segments) where the free radicals cause the cell and cause the tissue damage. Free radicals, besides attack on cell membranes (bilamillar lipid protein complex), intracellular organelle, they can break strands of DNA (the genetic material in the cell nucleus).

The broken strands of DNA are where the chemicals proved to cause cancer by forming free radicals. From the above description, it is obvious where the ROS generated due to the light perception. The ROS associated metabolic processes play an important role in age related macular degeneration. Our invention of insulin used with other therapeutic agents will help to curtail ROS production and damage. This is similar to the insulin protective effects on the myocardium of the heart in the cardioplegic solutions after open heart surgery, heart attack, and driving the potassium in or out of the cells using GIK infusion.

Experimental studies show that the cone and rod photoreceptors remaining in many age related macular degeneration patients functions normally for their numbers with the amounts remaining visual pigment which the belief support an idea that these photoreceptors can be rescued (Eliot L. Berson. Age related macular degeneration. The Friedenwald Lecture Investigative Ophthalmology and Visual Science, April 1993, Vol. 34, No. 5, 1659-1676).

Our invention using insulin ophthalmic preparations with nurticeuticals and other therapeutic agents can rescue these remaining photoreceptors, prevent their progression to apoptosis, maintain the remaining vision perceived by these photoreceptors, and prevent the progression of age related macular degeneration. ARMD can lead to total blindness. Risk or hazard factor investigation analysis of well-defined populations studied over time may reveal ameliorating or aggravating factors associated with the course of the disease. The possible implications for prophylactic therapies used in our invention described herein.

Before using described inventive methods and examples; a thorough examination of the affected patient's eye is in order. The examination of the eye may include: 1. Acuity testing, 2. Biomicroscopy, 3. intraocular pressure (10P), 4. Ophthalmoscopy, 5. Color vision test, 6. Tear osmolality, 7. Schimer's test, 8. Tear film breakup time (tBUT), 9. Test for Superficial punctate keratitis (SPK), 10. Fluorescein and Rose Bengal staining (RBS) of BV of the retina, as well as cornea, conjunctiva, and eyelids, 11. Slit-lamp examination of the conjunctiva, cornea, anterior chamber, iris, and lens, 12. The Ocular Surface Disease Index (OSDI), 13. Microscopic examination of the tear filament, 14. Maturation index (a Papanicolaous stained sample of conjunctival epithelium). 15. The most important test for retinitis pigmentosa is electroretinogram (ERG) to measure the function of the photoreceptors. 15. A normal view of an Amsler grid and (b) the distortion of the straight lines (metamorphopsia) and black spot (scotoma), as might be seen by a patient with neovascular age related macular degeneration should be tested.

In addition, a complete physical examination with blood test for thyroid, parathyroid, growth hormone, insulin, IGF-1, FSH, LH, cortisol, estradiol, and testosterone levels, electrolytes, blood cell count, cholesterol levels, ESR, and a urine sample for pregnancy when this is deemed necessary when the patient is of childbearing age.

The following diagrams describe the structure of the eye, and explain the route of absorption, movement, diffusion, and transportation of insulin and other therapeutic agents instilled in the conjunctival sac topically for the treatment of age related macular degeneration (ARMD).

FIG. 1, is a schematic diagram of the longitudinal section of the eye 100 and the location of the macula lutea 105 (boxed in) and its histological structures 106-112 affected by the AMRD. This diagram is showing the route of delivery of Insulin and other therapeutic agents to the macula, the site of ARMD from the conjunctival sac. It shows the eye dropper 101 for applying the therapeutic agents to the conjunctival sac 102. From the conjunctival sac 102 the therapeutic agents 103 are absorbed by choroidal vascular system 104 through the subconjunctival blood vessels, intrascleral BV and transported to the choirdal BV 104 and suprachoroidal space 107.

They reach the macula lutea 105 and fovea centralis (boxed space). The insulin from the conjunctival sac reaches the choroidal BV 108 below the suprachoroidal space 107 and sclera 106. From these large BV of the choroid 108, the insulin and other therapeutic agents enter the fenestrated choriocapillaries 109. The insulin leaks through the choriocapillaries 109 to Bruch's membrane 110 and transported to pigment epithelium 111 to the photoreceptors 112 of the fovea centralis and the structures surrounding the fovea and macula lutea.

The therapeutic agents 103 deposited in the conjunctival sac 102 enters the anterior chamber aqueous humor through the episcleral and intrascleral arteriovenous plexus which passes through the uveoscleral meshwork, Corneoscleral meshwork, Juxtacanalicular or cribriform trabecular meshwork, Schlemm's canal, Corneal endothelium joining the trabecular meshwork, Longitudinal and circular fibers of the ciliary muscles; muscle fibers of the iris, Scleral sinus vein, Scleral Veins, Suprachoroidal space 107 between choroidal BV 108 and sclera 106. The conjunctival sac 102 (fornix) where the therapeutic, pharmaceutical, biochemical and biological agents or compounds are deposited to be transported to the Macula Lutea 105 (boxed in) and its histological contents (arrow) 106-112 of the retina passing through the anterior chamber, irido-scleral angle, ciliary body, choroid plexus projecting from the ciliary body, choroid 104, which all play an important role in transporting the insulin and therapeutic agents to the Macula, the site of ARMD.

This diagram illustrates how easy it is for the insulin and other selected therapeutic agents to reach the afflicted ARMD site 105 from the conjunctival sac 102. The arrow marker 103 indicates the site of entry of therapeutic agents passing through various above described structures of the anterior segment of the eye to be effective in the treatment of ARMD acting to prevent further progression, and curing the condition. This method therapeutic agent's delivery prevents the therapeutic agents circulating all over the body through the systemic circulation to reach the site of AMRD with their associated adverse effects if taken orally or parentarily.

FIG. 2 is a schematic view of the longitudinal section of the part of the eye 200 and the location of the macula lutea 214 and its histological structures in ARMD compared to healthy retina 215. This diagram shows the location of pathology of the ARMD in the retina, pigment epithelium and choroidal blood vessels (BV). The diagram shows the pathology of the AMRD of the fovea centralis 214 compared to the rest of the healthy retina 215.

The diagram shows the sclera 201, large BV of the choroid 202 and the choriocapillaries 203 and 210. Note the invasion of the neochoriocapillares 205 through the Bruch's membrane 204 and retinal pigment epithelium 206 (RPE), with disruption of cones outer segment 207. Notice the Drusen 217 and the complex network of neochoriocapillares 205 with its edematous inflammatory fluids pushing the photoreceptors 207 from the RPE 206 with retinal detachment with bulging of the outer limiting membrane 208.

Take notice of the retina, adjacent to ARMD site is normal with normal suprachoroidal space 209, choroid 210. RPE 206, rods 211, and the Muller cells 212 that contribute to the formation of outer limiting membrane 208. The therapeutic agents of our invention insulin administered through the conjunctival sac reaches the site of neochoriocapillares 205 and Drusen 217 through the choroidal vascular system 202, 203, 205 and suprachoroidal space 209.

FIG. 3 is a diagrammatic presentation 600 showing the route of drainage of the lacrimal fluid and therapeutic agents shown as bubbles from the conjunctival fornix (sac) 601 to the nasal mucosa 605 and illustrates a method to prevent the agents from entering the nasal mucosa. A simple method applying the finger pressure 604 at the medial eye angle and nasal junction. The location of the lacrimal punctum, canaliculi 602, 603 and lacrimal sac with a finger 604 will prevent the therapeutic agents drainage to the nasal cavity and the nasal mucosal absorption 605, and their associated systemic adverse effects.

THE FOLLOWING ARE THE EXAMPLES OF USING OUR INVENTION OF INSULIN AND/OR IGF-1 BIOLOGICAL FACTORS ALONE OR IN COMBINATION WITH KNOWN THERAPEUTIC, PHARMACEUTICAL, BIOCHEMICAL, NUTEICUETICAL, AND BIOLOGICAL AGENTS OR COMPOUNDS TO TREAT AGE RELATED MACULAR DEGENERATION AND OTHER ASSOCIATED OCULOPATHIES.

### Example 1

Select the patient; establish the type of Age related macular degeneration and its etiology, if possible, which the person is suffering from. The complete and thorough examination of the eye as described above is imperative. Record the preliminary examination results on the patient chart. The patient should be examined for any corneal, conjunctival, and retinal BV afflictions by using marker dyes and other ophthalmological examinations. Position the patient in a supine posture or sitting with the head hyper extended with a support. Using a dropper or dropper bottle containing the insulin formulations. Instill two or three drops of insulin preparation in each eye lower lid fornix and/or everted upper eyelid (FIG. 1). Apply slight pressure at the nasal angle of eye on the nasolacrimal canaliculi-sac-duct system to prevent leaking of the therapeutic agents to the nose to avoid systemic absorption (FIG. 3).

The adverse effects can be prevented or minimized using the method shown in the FIG. 3. The patient must remain stationary for 2 to 5 minutes in supine position with head extended. The patient can resume the desired posture after the patient has been stationary for 2 to 5 minutes. These instructions should be given to all the patients. The patient or the caregiver should be trained to apply the ophthalmic drops using sterile methods for the treatment of age related macular degeneration with our inventive eye drops which the eye drops contain insulin. The insulin ophthalmic therapeutic drops are used before going to bed and after getting up from bed in the morning, after taking a shower as well as before taking a nap in the afternoon if possible.

### Example 2

Follow the instruction as described in the above EXAMPLE 1.

If the age related macular degeneration is associated with keratoconus sicca, use a topical FDA approved emulsion of cyclosporin for treating the associated condition (Restasis™, Allergan, Inc., and Irvine, Calif.). The emulsion is a mixture of cyclosporin combined with a higher fatty acid glyceride, like castor oil, and a surface active agent, such as polysorbate 80, and an emulsion stabilizer, such as a cross-linked polyacrylate. This acts by decreasing the inflammation on the eye surface (probably eye lid tear glandular system).

The emulsion helps to increase the production of healthy tears. However, treatment with an emulsion containing oily droplets can result in eye irritation or a clouding of the visual field. The emulsion may delay the absorption of insulin. The oily consistency of this preparation makes the active ingredient less bioavailable. Restasis is not appropriate for immediate relief for an uncomfortable irritated eye as the results may take up to 6 months for maximum improvement (source: The Eye Digest). The addition of insulin will make the preparation more effective which the Insulin enhances the uptake of cyclosporin, and augment/amplify the effects of the cyclosporins in the preparation.

This biological effect requires less cyclosporine which insulin can be added in the final cyclosporin preparation at the same time. There will be a decrease of time needed inside the afflicted cells to achieve the desired effects. The use of insulin before or with the preparation will enhance the activity of Restasis. The insulin will cause the Restatasis to become more effective within days instead of months due to augmentation/amplification effects of insulin. We prefer to use water soluble solution of cyclosporin as described. Then apply one drop of aqueous cyclosporin in water soluble eye preparation as formulated in the invention U.S. Patent Application Publication Number: US 2010/0016219 AI. Insulin can enhance the uptake of water soluble cyclosporin more efficiently than oil soluble preparations which it can augment and amplify the effects of the cyclosporins on the structures involved in development of age related macular degeneration associated with dry eye syndrome and other oculopathies.

### Example 3

Follow the instruction as described in the above EXAMPLE 1. If the men and woman suffer from age related macular degeneration with dry eyes syndrome due to estrogen and testosterone deficiency they can be treated with estrogen and testosterone ophthalmic drops with insulin. Androgens are believed to be trophic factors for various glandular and neuronal tissues including the retina. The androgens exert potent anti-inflammatory activity through the production of transforming growth factor beta (TGF-beta), suppressing lymphocytic infiltration, inflammatory response in the pigment epithelium, and the retina and the associated blood vessels.

The eye drops containing testosterone can be prepared and the drops can be used after pretreatment with insulin. The ophthalmic drops can be prepared using testosterone (androgen), DHEA—a mild androgen, cyclosporin. Insulin can be used to treat age related macular degeneration with the dry eyes syndrome, Sjogren's syndrome, and KCS at the same time. Our preliminary studies indicate, that the preparation for these syndromes, are easy to prepare. These ophthalmic eye preparations with insulin are used to treat Age Related Macular Degeneration associated with these oculopathies.

### Example 4

Follow the instruction as described in the above EXAMPLE 1. Previous, investigations demonstrated that bendazac prevents protein denaturation produced by U.V. rays. The bendazac is capable of attenuating the biological effects of sun radiations and the tissue associated with ROS on the retina, RPE, and choriocapillaries. This possibility was confirmed by the recent observation that bendazac has a protective effect on photo-oxidative processes Linked to free radicals involved in the age related macular degeneration.

The photosensitizing effect seemingly linked to the formation of free radicals (ROS) as described above where free radicals damages the photoreceptors. The ophthalmic solution of 1% lysine salt of bendazac can be used with insulin. Our invention enhances therapeutic agents to reach the site of pathology in the retina. Lysines salt of bendazac at the oral dose of 500 mgs/three times daily for a period of 6 months are administered when using insulin and bendazac ophthalmic preparations to augment the therapeutic agent's effect.

### Example 5

Follow the instruction as described in the above EXAMPLE 1. Then use the pharmaceutical kit for treatment of age related macular degeneration containing the enzymes glutathione peroxidase (Enzyme A), prolidase (Enzyme B), glucose-6-phosphate dehydrogenase (Enzyme C); optionally, aldose reductase (Enzyme D) in aliquot parts and interactive quantities appropriate, for administering ophthalmic drops for approximately three consecutive days, at monthly intervals, for about three months for each eye as disclosed U.S. Patent Application Publication Number: 2006/0134088 AI. These therapeutic agents are used in combination with insulin before, during, or after application of the ophthalmic drops.

### Example 6

Follow the instruction as described in the above EXAMPLE 1. U.S. Pat. No. 7,037,943 B2 discloses a method for treating or preventing retinal pathology or injury by placing a retinal stimulating substance in the eye between the internal limiting membrane and the retina, which the internal limiting membrane is the target site for the substance. The substance may be an implant that provides electrical stimulation to adjacent ganglion and neurofiber cells.

Alternatively, the substance may be a pharmaceutical substance to stimulate the retina. In addition to providing direct contact where the substance has its target he method obviates the need for artificial structures which the structures are tacks or adhesives which the artificial structures may cause retinal bleeding or traction. Our invention of using insulin ophthalmic drops with semi surgical therapeutic procedure will partake in the augmentation-amplification effects of surgically introduced therapeutic agents to contain the disease of age related macular degeneration and other oculopathies much more effectively and heal the surgical intervention site much faster in addition.

### Example 7

Follow the instruction as described in the above EXAMPLE 1. U.S. Pat. No. 5,948,801 discloses the use of Brinzolamide as eye drops, systemically between 250 to 1000 mg orally, or intravitreal up to 10 mg per eye or periocular up to 50 mg per eye to treat retinal edema. We want to incorporate Brinzolamide ophthalmic drops incorporated to treat oculopathies of various kinds including age related macular degeneration combined with insulin ophthalmic drops to maintain the integrity of RPE cell layer by decreasing the edema where the relief of the edema can play a role in alleviating the condition of age related macular degeneration.

### Example 8

Follow the instruction as described in the above EXAMPLE 1. U.S. Pat. No. 6,716,835 BI discloses a method of retarding degeneration of retinal photoreceptors in patient afflicted with age-related macular degeneration. A therapeutically effective amount of a compound selected from the group consisting of calcium channel blocker compounds and/or cyclic GMP-dependent channels, namely diltiazem, for treating retinal pathologies, and more particularly retinal diseases caused by degeneration of visual receptors. The diltiazem can be formulated as ophthalmic preparation with insulin to be used and to treat age related macular degeneration in our invention.

### Example 9

Follow the instruction as described in the above EXAMPLE 1. U.S. Patent Application Publication Number: 2001/0049369 AI demonstrates that brimonidine tartrate, a potent alpha-2 adrenergic receptor agonist, applied topically to the eyes can prevent photoreceptor cell degeneration. The Muller cell associated with degenerative signs in an in vitro model of retinal degeneration and retinal detachment. Brimonidine allowed for the formation of highly structured photoreceptor outer segments, prevented the expression of stress markers in Muller cells, and preserved the expression patterns of Muller cell markers of proper cell to cell contact and differentiation.

Ultra structural studies indicated that Brimonidine favored the formation of cell to cell junctions between photoreceptor cells. The Muller cells with the cell to cell junctions indicate that this phenomenon is associated with the exertion of the neuroprotective effect. The results suggests that brimonidine compounds may be utilized as an effective therapeutic agent for early and late onset retinal degenerations caused by defects in photoreceptor cells, Muller cells, or both, as an adjuvant to therapeutic success in retinal detachment surgery or macular translocation surgery for age-related macular degeneration and age related macular degeneration.

This therapeutic agent has been used for treatment of chronic open angle glaucoma also. Our inventive method uses brimonidine with insulin ophthalmic drops to enhance its uptake for augmentation/amplification effects on the photoreceptors cells, and other components of retina to prevent oculopathies including age related macular degeneration.

### Example 10

Follow the instruction as described in the above EXAMPLE 1. U.S. Patent Application Publication Number: 2009/0060980 A1 discloses a novel method of treatment for retinal diseases and conditions including age-related macular degeneration, genetic-based retinal degenerations, and retinal detachment. A novel glycan binding protein is thought to be a cell surface receptor that the cell has been discovered in the retina. The retinal glycan binding receptor is shown to play an important role in promoting assembly of outer segment (OS) membranes by the photoreceptor cells of the eye.

This is a process that is essential for vision. Based on the finding, certain sugars can bind with very high affinity to the retinal glycan receptor which the sugars stimulate the retinal glycan function. The invention provides novel therapeutic agents for treatment of retinal diseases that are multivalent N-linked glycans. Preferred pharmaceutical compositions in accordance with the present invention comprise active agents having the general formula: (Gal-GlcNAc), -Man3₃-GlcNAc_{z}, where n is 1-4. Particularly preferred multivalent glycans are galactosylated, biantennary (NA2), also, galactosylated, triantennary (NA3) oligosaccharides. We want to incorporate insulin with our invention so it can be used with these oligosaccharides to treat age related macular degeneration and other oculopathies.

### Example 11

Follow the instruction as described in the above EXAMPLE 1. The presently disclosed U.S. Patent Application Publication Number: 2009/0053816 AI provides methods of diagnosing retinal disorders in subjects by measuring hemoglobin and measuring modified hemoglobin in the subjects. The presently disclosed subject matter provides methods of treating retinal disorders in subjects by decreasing hypoxia in retinal tissue of the subjects through modulation of hemoglobin levels and activities in the retinal tissue.

Our inventive method uses insulin ophthalmic instillation to the method of modulating hemoglobin as described in the above patents will enhance the activity and will reduce the likelihood of hypoxic damage of photoreceptors, where the hypoxic damage leads to age related macular degeneration development or aggravates the existing disease.

### Example 12

Follow the instruction as described in the above EXAMPLE 1. Antibodies are proteins that the antibodies are generated by the immune system's white blood cells. The antibodies circulate in the blood which the antibodies attach to foreign proteins called antigens in order to destroy or to neutralize them which the antibodies help rid the systemic infection or eliminate foreign proteins harmful to the body cells. Monoclonal antibodies are laboratory created or fashioned substances that the antibodies can locate. The antibodies bind to specific molecules such as tumor necrosis factor (TNF) which the TNF is a protein involved in causing the inflammation and the damage of autoimmune diseases.

There are many MAB such as: Remicade™, Etanercept, Embrel™, and Humira™. The TNF and anti TNF agents are on the market to treat autoimmune bodies. Etanercept is a drug that the drug is used to treat autoimmune diseases by interfering with the tumor necrosis factor (TNF, a part of the immune system) by acting as a TNF inhibitor. This is given 25-50 mg. Humira administered by injection is produced from human proteins. The newest monoclonal protein to be approved for the treatment of rheumatoid arthritis is Rituxan. Infliximab (Remicade) is a chimeric mouse/human monoclinal antibody given by intravenous infusion the monoclonal protein works by binding to tumor necrosis factor alpha (TNFα). Several new monoclonal antibodies are in the development stage to treat autoimmune diseases.

Multiple monoclonal antibodies are currently under investigation for the treatment of age related macular degeneration (Meijer J M, Pijpe J, Bootsma H, Vissink A, Kallenberg C G (June 2007). “The future of biologic agents in the treatment of “Sjögren's syndrome”. Clin Rev Allergy Immunol 32 (3): 292-7). All TNF inhibitors are immunosuppressants. We formulate Etanercept (Embrel) using no more than 200 μg per ml of ophthalmic solution which these results in 10 μg per drop instilled. The final solution will have insulin as described above to reduce the nonspecific inflammatory processes in the photoreceptors in age related macular degeneration caused by ROS. The patient should use the insulin and MAB preparations once or twice a day. The dose of MAB used in our invention is minuscule.

We must take into account any contradications with tuberculosis or tumors while using these biological therapeutic agents with our insulin invention. Antiangiogenesis MABs may be used to treat neovascularization from choriocapillares (wet ARMD) with insulin ophthalmic drops.

### Example 13

Follow the instruction as described in the above EXAMPLE 1. The hyaluronic acid (HA) is produced by fermenting the bacterial strain Bacillus subtilis. It is the world's first pure HA that is 100% free of animal-derived raw materials and organic-solvent remnants. Hyaluronic acid is a novel viscosity enhancer for use in topical eye care formulations which hyaluronic acid is filterable. The hyaluronic acid is heat stabile with pH (0.1% solution) 6.0-7.5 which this is desired to treat age related macular degeneration and other oculopathies.

The HA can be a key ingredient for topical ophthalmic formations. The hyaluronic acid is a natural compound which the compound is biocompatible, non-immunogenic, and biodegradable. This compound is one of the most hygroscopic molecules found in nature. The hydrated hyaluronic acid can contain up to 1,000-fold more water than its own weight. These exceptional water retention properties result in enhanced hydration of the corneal surface which retain the active therapeutic agents to be slowly released to be absorbed and transported to the site of Age related macular degeneration.

Moreover, applications of ophthalmic formulations containing HA reduce tear elimination which HA enhances pre corneal tear film stability. The HA has a useful property against age related macular degeneration. The muco-adhesivity of hyaluronic acid provides effective coating and long lasting protection of the cornea and conjunctival sac due to the extended stay, water retention quality, and accommodation times on the ocular surface. When topically instilled on the eye with insulin, HA promotes physiological wound healing by stimulating corneal epithelial migration and proliferation of keratocytes. HA enhances the healing of photoreceptors, RPE, and Bruch's membrane which HA acts as therapeutic agents for treatment of Age related macular degeneration with other oculopathies. HA has the viscosity-enhancing agent of choice, decreases the drainage rate of ophthalmic solutions where the HA allows the insulin to be absorbed into deep eye structures including the choroid and the retina. Our invention of using insulin before and after the application of the HA with or without other anti age related macular degeneration therapeutic agents combining with insulin in the final formulation can effectively prevent, curtail, and cure the age related macular degeneration associated with or without other oculopathies.

### Example 14

Follow the instruction as described in the above EXAMPLE 1. Mitoxantrone (Novantrone) is a chemotherapeutic drug that the drug works by suppressing the immune system. Mitoxantrone is used to slow the worsening of neurologic disability and to reduce the relapse rate in patients with clinically worsening forms of relapsing-remitting and secondary progressive MS. Mitoxantrone is a DNA-reactive agent, that agent intercalates into deoxyribonucleic acid (DNA) through hydrogen bonding, where the Mitoxantrone causes crosslink's and strand breaks. Mitoxantrone interferes with ribonucleic acid (RNA).

Mitoxantrone is a potent inhibitor of topoisomerase II, an enzyme responsible for uncoiling and for repairing damaged DNA especially in photoreceptors cells of age related macular degeneration. Mitoxantrone can be prepared in doses of 100 μg/ml by premixing with insulin. These drops can be effective in autoimmune related age related macular degeneration.

### Example 15

Follow the instruction as described in the above EXAMPLE 1. Corticosteroids are the most commonly used treatment for autoimmune diseases, allergic conditions, insect bites, septic shock, and many other conditions including age related macular degeneration. The corticosteroids are given to reduce the inflammation. Examples included are oral prednisone and intravenous methyl prednisolone. Lotemax, an ophthalmic corticosteroid, targets inflammation with a unique site-active mechanism of action. Structural modifications associated with an ester ophthalmic steroid, which Lotemax make highly lipid soluble, enhancing the penetration into cells, and enabling Lotemax to exert anti-inflammatory activity within the eye. Pre-treating with insulin or combining with insulin ophthalmic drops can enhance the uptake of these corticosteroids and relive age related macular degeneration and other autoimmune afflictions of the eye. The insulin with steroid attenuates the effects of ROS mediated photoreceptor and RPE cells damage, stabilizes the membranes of the photoreceptors and RPE cells, and their organelle which restores function and health.

### Example 16

Follow the instruction as described in the above EXAMPLE 1. Studies on experimental animals retinal pigment epithelium (RPE) showed, that RPE actively secretes sodium and calcium into the retinal space, which the space absorbs chlorine and maybe bicarbonate and potassium. This activity could be important in controlling the ionic milieu in the outer retina. (Miller, et al., “Active Transport of Ions Across Frog Retinal Pigment Epithelium,” Experimental Eye Research, 25:235-248 (1977)). Acetazolamide have been used in glaucoma and has application in preventing or slowing the spread of retinal detachments or hastens re-absorption of subretinal fluid if age related macular degeneration is associated with uveal and macular edema.

U.S. Pat. No. 5,948,801 discloses methods for preventing and treating retinal edema with Brinzolamide similar to Acetazolamide are disclosed. It has been shown to be effective in the treatment of chronic macular edema associated with age related macular degeneration (Gerald A. Fishman, M D; Leonardo D. Gilbert, C O T; Richard G. Fiscella, RPh, M P H; Alan E. Kimura, M D; Lee M. Jampol, M D. Acetazolamide for Treatment of Chronic Macular Edema in age related macular degeneration. Arch Ophthalmol. 1989, 107(10):1445-1452). Acetazolamide is more effective improving the macular edema compared to brindorzolARMSe. Photoreceptors dysfunctioned in a roundabout way. Age related macular degeneration may be related to retinal pigment epithelium edema resulting in disruption of photoreceptors function. In our invention we want to use ophthalmic drops containing Brinzolamide, and/or Acetazolamide with insulin in age related macular degeneration to relieve swelling of the pigment epithelium which the insulin would restore the function to maintain the photoreceptors cells.

### Example 17

Follow the instruction as described in the above EXAMPLE 1. There are two types of fatty acids needed for health and these fatty acids are used by millions every day as health nurticeuticals supplement. One is Omega 3 and the other is Omega 6. Omega 3 fatty acids include: Alpha-linolenic acid (ALA), Eicosapentaenoic acid (EPA), Docosahexaenoic acid (DHA). The Omega 6 fatty acids include: Linoleic acid (LA), Gamma linolenic acid (GLA), Dihomo-gamma-linolenic acid (DGLA), and Arachidonic acid (AA). Gamma-linolenic acid (GLA) is an omega-6 fatty acid found mostly in plant-based oils. GLA is considered an essential fatty acids and antioxidants.

These fatty acids need to be supplemented. They are necessary for human health which the body isn't capable of producing the fatty acids. Hence, the fatty acids have to be obtained through every day food. They are the omega-6 fatty acids with omega-3 fatty acids, also, known as polyunsaturated fatty acids (PUFAs). These play a vital role in brain function, its normal growth and development, which the retina is part. They help to stimulate skin, hair growth, maintain bone health, regulate metabolism, and maintain the reproductive system. To maintain health, the ratio of omega-6 to omega-3 fatty acids consumed should be the ratios of 10:1 to 5:1 previously, it was 15:1.

The latest studies show that the approximately 8% of the brain's weight is comprised of omega-3 fatty acids (DHA and EPA) (O'Brien J S, Sampson E L. Lipid composition of the normal human brain: grey matter, white matter, and myelin. (J Lipid Res. 1965 October; 6(4):537.44). The building block for an estimated 100 billion neurons (Chang C Y, Ke D S, Chen J Y Essential fatty acids and human brain. Acta Neurol Taiwan. 2009 December; 15(4):231-241). Omega 3 fish oil contains two active ingredients: EPA (Eicosapentaenoic Acid) and DHA (Docosahexaenoic Acid). They are interconvertible in the brain. They play a host of vital roles in neuronal structure and function, protecting the neural structure from oxidative damage, inflammation, and the cumulative destruction inflicted by other chronic insults.

The retina is an extension of the brain with millions of photoreceptors and other neurons. The Omega 3 fatty acids can protect the photoreceptors from oxidative damage, inflammation, and the cumulative destruction inflicted by other chronic insults where they do with CNS. Embedded in the omega-3 DHA-rich retinal photoreceptors and neuronal membranes are numerous proteins with complex molecules required for electrochemical transmission, signal reception, and transduction. Scientists have recently shown that the precise balance of fatty acids in brain cells help to determine whether a given nerve cell in the retina will be protected against injury, inflammation, or whether it will succumb to the injury (Julius Goepp. Omega 3 Fatty Acids increase Brain Volume while reversing many aspects of neurologic aging. Life Extension, August 2010, Pages 56-61).

A remarkable animal study has revealed that omega-3 fatty acids halt the age-related loss of brain cell receptors vital to memory production which the fatty acids show potential for increasing neuronal growth (Dyall S C, Michael G I Michael-Titus A T. Omega-3 fatty acids reverse age-related and Omega-3 fatty acids decreases in nuclear receptors and increase neurogenesis in old rats. J Neurosci Res. 2010 Mar. 24). Animal studies suggest that oral supplementation with DHA may enhance the formation of new synapses and their vital dendritic spines. The supplementation can improve cognitive function (Wurtman R I, Cansev M, Ulus R H. Synapse formation is enhanced by oral administration of uridine and DHA, the circulating precursors of brain phosphatides. J Nutr Health Aging 2009 March; 13(3): 189-97). Again, the retina being part of the brain and the brains' extension, DHA, and EPA will have the same effect on the photoreceptors and other neurons of the retina. These fatty acids can improve their synapses function, prevent damage to the vision caused by age related macular degeneration, and other oculopathies.

Omega 3 significantly reduced levels of inflammatory cytokines circulating in the blood. This suggests that the brain and retinal tissue inflammation can be alleviated or toned down in age related macular degeneration and other oculopathies. The molecular basis for this early intervention strategy lies in the photoreceptors cellular pathophysiology at the core of the age related macular degeneration: omega-3 treatment of cultured brain cells suppresses many of the early signs of damage triggered by the inflammatory protein known which this includes the beta amyloidal of Alzheimer's (Ma Q L, Yang F, Rosario E R, et al. Beta-amyloid oligomers induces phosphorylation of tau and inactivation of insulin receptor substrate via c-lun N-terminal kinase signaling: suppression by omega-3 fatty acids and curcumin. J Neurasci. 2009. 15; 29 (28):9078-89).

Most omega-6 fatty acids in our diet come from vegetable oils in the form of linoleic acid (LA). Salmon and related fish are a rich source of omega complexes EPA and DPA (Docosapentaenoic acid). 33% of the long chain Omega-3 fatty acids circulating in human blood is attributable to DPA. The BV wall can convert EPA to DPA as the effective agent. The body converts linoleic acid to GLA and then to arachidonic acid (AA). GLA can be obtained from several plant-based oils including evening primrose oil, borage oil, and black currant seed oil. A healthy diet should contain a balance of omega-3 and omega-6 fatty acids. The omega-3 fatty acids help to reduce inflammation in photoreceptors.

Our invention of using insulin ophthalmic drops with omega-3 fatty acids can be applied to the eyes along with oral intake. They can be prepared with mixing of Vitamin A. The patient takes orally DHA 1,700 mg combined with 600 mg EPA omega-3 fatty acid (DPA-EPA). The patient should wait 30 to 60 minutes for the DHA-EPA to be absorbed and to reach high plasma levels. Then insulin drops should be applied to the eyes one hour later which the insulin will enhance the uptake of omega 3 from the choriocapillares by photoreceptors. The insulin will make the omega-3 more effective in the treatment of age related macular degeneration and other oculopathies. Insulin and Omega 3 ophthalmic drops can be formulated to treat ARMD.

### Example 18

Follow the instruction as described in the above EXAMPLE 1. There is high incidence keratoconjunctivitis sicca in postmenopausal women with symptoms ranging from mild foreign body, pain and even visual loss due to ocular surface abnormalities including age related macular degeneration. The use of conjugated estrogens decades ago to treat KCS was indicated (Bohigian, G. Handbook of External Diseases of the Eye (Alcon, Inc.) 1980, p. 79). U.S. Pat. No. 5,041,434; U.S. Pat. No. Re. 34,578; and 6,096,733 describe the use of estrogens. The latter patent disclosed very small doses of 17-β-estradiol compounded with polysorbate 80 (USP), povidone (USP) (K-30 type), hydroxyethylcellulose (USP), sodium chloride (USP), disodium EDTA (USP), benzalkonium chloride (USP), dilute HCL for pH adjustment, and purified water (USP) qs. As described in our invention pre-treating the affected eyes with insulin or adding to the above preparation of estradiol eye drop can enhance the local therapeutic effect by insulin mediated augmentation-amplification effects. This invention will provide the needed relief much faster without systemic effect if the condition is associated with age related macular degeneration.

### Example 19

Follow the instruction as described in the above EXAMPLE 1. The symptoms of an eye allergy are mild to moderate where allergies can be severe during early spring and the beginning of fall. Self treatment to avoid allergens, are to irrigate the eyes with saline (salt solution), to place the ice packs, and the cold water compresses on eyes which this may not be effective in a severe case. The medical treatment is needed to relieve them of the age related macular degeneration associated with severe allergic conjunctivitis. Conjunctivitis may benefit from specific allergen immunotherapy (desensitization) which the therapy is usually effective. Most commonly used and prescribed medications are: levocabastine (brand name Livostin); antihistamines (antolozine) with a medicine that constricts blood vessels (naphazoline, phenylephrine); sodium cromoglycate (4%); non-steroidal anti-inflammatory (NSAID) eye drops; and steroids (hydrocortisone, Dexamethasone, prednisolone). Eye drops containing anti allergic, vasoconstrictors, and cortisone, can be used long term to treat age related macular degeneration with allergic conditions. The drops with insulin applied before the use of the above described therapeutic agents. Our experimental data using insulin with vasoconstrictors and anti allergic therapeutic agents such as corticosteroids supports that the allergic condition is relieved rapidly. The red eye disappeared with prolonged effect when insulin was added to the ophthalmic therapeutic agents which the insulin can adversely affect the age related macular degeneration.

### Example 20

Follow the instruction as described in the above EXAMPLE 1. Testosterone has trophic effect on the neurological structures. Studies has shown subjectively the patients felt better when DHEA ophthalmic drops were used compared to the artificial tears or testosterone as artificial tears (Connor CG, and Fender J. Comparison of Androgenic Supplemented Artificial Tears. Invest Ophthalmol V is Sci 2002; 43: E-Abstract 66; Schaumberg D A, Sullivan D A, Dana M R. Epidemiology of Age related macular degeneration. Adv Exper Med Biol 2002; 506: 989-998. Schaumberg D A, Sullivan D A, Buring J E, Dana M R. Prevalence of Age related macular degeneration among US women. Am J Ophth 2003; 136:318-326).

These studies supports the previous studies by Notion and Sullivan that say the addition of androgenic hormones to artificial tears benefit various oculopathies. DHEA is known as dehydroepiandrosterone. This is a steroid hormone produced by the adrenal glands where the DHEA is converted to other hormones like estrogen and testosterone. DHEA is a steroid hormone produced naturally by the adrenal glands that has 5% of the androgenic activity of testosterone. Our invention relates the use of testosterone or DHEA eye drops with insulin. Use the insulin drops before the application of the androgenic eye preparation. These hormonal eye drops in combination with insulin can be prepared and used as ophthalmic drops to treat these conditions associated with age related macular degeneration.

### Example 21

Follow the instruction as described in the above EXAMPLE 1. A method of topically instilling insulin drops to a person or animals conjunctival sac to treat age related macular degeneration with administration of insulin. The insulin enhances their uptake. The insulin has therapeutic activity by entering into afflicted structures in the eye. This can be combined with uptake facilitators such electroporation, iontophoresis, sonophoresis, vibroacoustic, vibration, and other physical (heat, magnetic force, radio frequency, microwave, laser lights etc.) methods with other appropriate therapeutic, biological, pharmacological anti-glaucoma, and retinal protectors. These agents combined with insulin therapy as described. These methods can be used as prophylaxis, to diagnose, prevent and to treat the above conditions.

### Example 22

Follow the instruction as described in the above EXAMPLE 1. Deferoxamine is a chelating agent used to remove excess iron from the body. Iron removed which the reduction reduces the damage done to various organs and tissues, like the liver, CNS, and retina. The damage that we saw in the retina can be due to excessive iron from the choroid and retinal blood vessels leaking excessive iron reacting with ROS where the excess damages the sensitive photoreceptors. The role of iron (metallobiology) in neurodegenerative disorders has long been implicated with particular attention given to iron. Iron is one of the most important redox metals which iron has been largely linked to senile toxicity and neurodegenerative disorders which the disorders are as follows: Alzheimer's, MS, and Parkinson's diseases and aging patients (Stankiewicz J M, Brass S D (2009) Role of iron in neurotoxicity: a cause for concern in the elderly? Curr Opin Clin Nutr Metab Care 12:22-9). The redox switching capability of iron from ferrous to ferric state, and vice versa, makes iron one of the most dangerous catalytic elements responsible for the retinal and other neurodegenerative process resulting in diseases and dysfunction. Iron generates free radicals where the free radicals are reactive with the oxygen species in the aged tissue as evidenced by higher heme oxygenase-I, which this contributes to increased susceptibility, to oxidative stress with aging (Hirose W, Ikematsu K, Tsuda R (2003). Age-associated increase in heme oxygenase-1 and ferritin immunoreactivity in the autopsied brain. Leg Med 5(Suppl.1):360-6).

The nerve tissue of the photoreceptors are exposed to the iron will not spare from the iron effects of neurodegenerative process. Biochemical events surrounding iron-mediated catalytic events which the biochemical events give rise to oxidative stress and free radical generation that the events damages photoreceptors in Age related macular degeneration. The damage is described and the damage is known as the Fenton reaction as indicated below:

Fe3+.O2→KO2-Fe2++O2 (Step I);

Fe2++H2O2-Fe3++OH₋+KOH (Step II)

Combining Step I and II: .O2−+H2O2→.HO−+O2

The role of iron in the neurodegenerative process which the retina is part of the nervous system can be best described in three distinct phases: 1. accumulation in choroidal blood vessel walls and Bruch's membrane, 2. invasion through the RPE from the Bruch's membrane, and 3. catalytic activity against the outer segment of the photoreceptors. A recent study shows that iron chelation can speed the healing of nerve damage in age related macular degeneration where iron chelation can reduced or curtailed ARMD. The use of deferoxamine as iron chelator with our invention insulin can have dramatic curing and/or curtailing effect on the MS, Alzheimer's, Parkinson's, ALS, dementia with Lewy bodies (due to deposits of alpha-synuclein inside the brain's nerve cells), metntal depression, stroke, PTSD, Autism, Chorea, and other degenerative and nondegenrative diseases of the CNS including senile brain atrophy. These conditions and any and all other CNS afflications can be treated without the dederoxamine; just by using insulin alone or with other therapeutic agents or measures.

Deferoxamine may modulate expression and release of inflammatory mediators in the age related macular degeneration as indicated in Fenton reaction by specific cell types, thus, reduce or stop the damage by our invention. Deferoxamine used with insulin of our invention along with ophthalmic drops can reduce the ROS oxidant damage, arrest, or delay the processes of Age related macular degeneration with or without neovascularization of the choriocapillaries. We have used this method to treat the CNS disease with good results. We have used the extract of Turmeric, called curcumin, with insulin as antioxidant with good results. Curcumin is safe and isn't toxic to the retina or the CNS.

### Example 23

Follow the instruction as described in the above EXAMPLE 1. Another drug available to treat autoimmune disease related to Sjogren's disease is an organo sulfur compound, anethole dithiolethione (ADT-trade name Sialor, sold over the counter in Canada) which has hardly any side effects. The ADT stimulates the secretion of saliva, in patients with autoimmune exocrinopathy (Sjogren's syndrome). Sialor alleviates the symptoms of xerostomia and xeroophthalmia.

We have used ADT 25 mg orally and ADT in nanograms concentration in liquid ophthalmic eye drops with success in these conditions, especially, those on chemotherapy, menopausal women, and chronic smokers with dry mouth and dry eyes conditions. There is secretory dysfunction associated with RPE and Muller cells which are needed for proper functioning of the photoreceptors by removing ROS. This can be one of the important non toxic oral and eye drops for the treatment of age related macular degeneration (Ben-Mandi M H, Gozin A, Driss F, Andrieu V, Christen M O, Pasquier C. Anethole dithiolethione regulates oxidant-induced tyrosine kinase activation in endothelial cells. Antioxid Redox Signal. 2000 Winter, 2 (4):789-99). Studies by Han et al show that ADT is more bioavailable lipid-based formulations, as sub-micro emulsion (SME) and oil solution prepared using short (SCT), medium (MCT) and long (LCT) chain triglycerides respectively. (Han S F, Yao T T, Zhang X X, Gan L, Zhu C, Yu H Z, Gan Y. Int J Pharm. Lipid-based formulations to enhance oral bioavailability of the poorly water-soluble drug anetholtrithione: effects of lipid composition and formulation. 2009 Sep. 8; 379(1):18-24. Epub 2009 Jun. 7.).

It is known that it pumps out the toxins of smoking from the respiratory lungs cells, making them healthy and reduce the chances of cancer. In the same fashion, it pumps out the toxic substances from the neovascularization of the choriocapillaries, RPE and from the photoreceptors thus, creating homeostatic physiologic media for their proper functioning, at the same eliminating the toxic substances that predispose to the development of ARMD. The emulsion or water soluble compound of ADT ophthalmic drops can be used after insulin drops. Insulin can be combined with the formulation to instill to the eye with one dispenser. The ADT is non toxic. ADT can be very efficacious in treating age related macular degeneration associated with or without dry eye syndrome.

### Example 24

Follow the instruction as described in the above EXAMPLE 1. Alagebrium (known as ALT-711) is the first drug to be clinically tested for the purpose of breaking the cross links caused by advanced glycation end products (AGEs), thereby, reversing one of the main mechanisms of aging. This has been seen in diabetics at an early age which glycation may be in age related macular degeneration resulting in build up Drusen. Drusen are yellowish-white nodular deposits found in the deeper layers of the retina. They comprise hyaline deposits or colloid bodies of Bruch's lamina of the choroid, and may not always affect vision.

Drusen are seen as a consequence of aging which can be found in the younger age group also. Drusen are often associated with ARMD with increased risk of visual loss. The drying seen in the diabetics and the aged can be related to AGEs due to carbohydrates binding to proteins including structural proteins, lipids, and DNA as seen in deposits of Drusen. This process can impair the normal function of organs that depend on flexibility and proper nutrition supply for normal functioning. AGEs cross links leads to loss of function of tissues and induces oxidative stress which AGEs reacts with molecules provokes the underlying component of inflammation. Hence, the Alagebrium eye drops in combination with Insulin can prevent AGEs formation, facilitate their removal, and reverse the disease state affecting the photoreceptors function. There may be relief from further development, advancement of age related macular degeneration, and cataract with diabetic retinopathy.

### Example 25

Follow the instruction as described in the above EXAMPLE 1. There isn't a definitive cure for age related macular degeneration. Another objective of our invention is to cure or curtail the Age related macular degeneration cases. The genes account for no more than 60% of all patients. The remainder has defects in unidentified genes. Findings of controlled trials indicate that nutritional interventions, including vitamin A palmitate and omega-3-rich fish, slow the progression of the retinitis pigmentosa disease in many patients. The findings indicate that our invention with the use of insulin, where these nutritional supplements can arrest and can cure about 40% of the patients, who don't show the genetic based photoreceptors apoptosis leading to age related macular degeneration.

### Example 26

Follow the instruction as described in the above EXAMPLE 1. Oral intake of Vitamin A, B₆, C, D₃, E, GLA, has been known to delay the progression of the retinitis pigmentosa so also age related macular degeneration. Vitamin E seems to play a role which works together with Vitamins A and D. Vitamin D is the only molecule that we create ourselves from sun light and turn into a hormone (OH25D). An amazing feat when you think about the process. Similarly, Vitamin A, obtained through the diet, is the other dietary lipid-based nutrient, that we turn into a hormone (retinoic acid) to be used by the photoreceptors pigment formation for light reception.

These supplements will help the condition of age related macular degeneration associated with retinitis pigmentosa. Insulin drops should be used 30 minutes to one hour after taking these supplements orally to enhance their uptake by the disease afflicted cells. In the eyes, these supplements circulate through the choroidal BV and are transported through the RPE to the outer segment of the photoreceptors. The progression of the disease can be reduced by the daily intake of 15000 IU (equivalent to 4.5 mg) of Vitamin A palmitate. Eleven-CIS Vitamin A can be used for treating this condition (Berson E L, Rosner B, Sandberg M A, et al. (1993). “A randomized trial of Vitamin A and Vitamin E supplementation for age related macular degeneration”. Arch. Ophthalmol. 111 (6): 761-72).

Recent studies have shown that the Vitamin A supplementation can postpone blindness by almost 10 years (Berson E L (2007). “Long-term visual prognosis in patients with Age related macular degeneration: the Ludwig von Sallmann lecture”. Exp. Eye Res. 85 (1): 7-14). Scientists continue to investigate possible treatments with less success. Vitamin A deficiency is more common than we realize resulting in malfunction of the photoreceptors. The Vitamin A rich foods are rarely eaten which the Vitamin A toxicity has been overblown to our profound immunological detriment. Vitamin A is necessary for optimal mucosal immunity and cell lining of all structures including the structures involved in the neovascularization of the choriocapillaries. Besides the health of cells, the Vitamin A is needed for the formation of photoreceptors pigment which the pigment is needed for vision. Vitamin A is a key nutrient in balancing the newly discovered pro-inflammatory cytokine, IL-17. Carotenes aren't an adequate or safe substitute for Vitamin A supplements in retinitis pigmentosa associated with age related macular degeneration. Carotenes and carotene rich foods like sweet potatoes, carrots, kale, spinach, turnip greens, winter squash, collard greens, cilantro, fresh thyme, cantaloupe, romaine lettuce, and broccoli have long been recommended and promoted as a substitute.

New research shows that the carotenes aren't efficiently converted to Vitamin A in 50% of the individuals. The carotenes can create cleavage products, which the products form free radicals, that these radicals interrupt Vitamin A's protective function. Hence, there is importance to take adequate amounts of Vitamin A where the patient doesn't depend upon its precursor of Carotenes. Our invention involves taking prescribed amounts of Vitamin A. The patient needs to wait for the Vitamin A to be absorbed which the absorption will take about one hour to occur. The blood concentration of Vitamin A reaches the peak level at one hour. Then, instill 0.5 to 1.00 units' insulin containing (per drop) in both eyes. The patient should wait 5-10 minutes for the insulin to be absorbed. The absorbed insulin in the retina will enhance the uptake of the circulating Vitamin A by photoreceptors where the effect will be therapeutic in curing or curtailing the retinitis pigmentosa and age related macular degeneration. Other vitamins such as Vitamin E and D₃ can be incorporated into Vitamin A ophthalmic drops.

### Example 27

Follow the instruction as described in the above EXAMPLE 1. Scientists at the Osaka Bioscience Institute have identified a protein, named Pikachurin which they believe could lead to a treatment for retinitis pigmentosa and can be used if associated with age related macular degeneration (Sato S, Omori Y, Katoh K, et al. (August 2008). “Pikachurin, a dystroglycan ligand, is essential for photoreceptor ribbon synapse formation”. Nat. Neurosci. 11 (8): 923-931). Our invention incorporates pikachurin along with insulin to make the treatment more effective in age related macular degeneration with retinitis pigmentosa.

### Example 28

Follow the instruction as described in the above EXAMPLE 1. Attempts have been made at University College London Institutes of Ophthalmology and Child Health and Moorfields Eye center to treat successfully the retinitis pigmentosa with stem cell transplant in mice with resulting in photoreceptor development with the necessary neural connections. Previously, belief was that the mature retina has no regenerative ability. The use of our invention with insulin ophthalmic drops augments rapid incorporation and differentiation of stem cells into the retina in any stem cell therapy. The insulin allows the stem cells to differentiate the photoreceptors, and the stem cells get connected to other retinal and central neurons. It can be combined with ARMD and retinitis pigmentosa treatment.

### Example 29

Follow the instruction as described in the above EXAMPLE 1. Studies involve the use of desmethyldeprenyl, a metabolite of the anti-Parkinson's drug, deprenyl for age related macular degeneration (W. A. Baumgartner. Etiology, pathogenesis, and experimental treatment of Age related macular degeneration. Medical hypothesis. Volume 54, Issue 5, Pages 814-824. May 2000). The rationale is based on an observation that desmethyldeprenyl exerts antiapoptotic activities in a variety of neurodegenerative disorders. The protective mechanism involves the over expression of the anti-apoptotic bcl-2 gene, leading to higher concentrations of bcl-2 proteins, which the proteins binds to mitochondria that the protein inhibits.

The trigger mechanism of apoptosis—is the opening of permeability transition pore (PTP), and the release of cytochrome C. At the same time, desmethyldeprenyl causes the under expression of the pro-apoptotic bax gene which via bax proteins facilitates the opening of the PTP. Both the anti-apoptotic and pro-apoptotic mechanisms appear to be mediated by the binding of desmethyldeprenyl to glyceraldehyde-3-phosphate dehydrogenase. Antiapoptotic effects can be generated by the parent compound, deprenyl when this is used daily in low concentrations of 1-2 mg/100 kg body weight. These conditions appear that the anti-apoptotic metabolite, desmethyldeprenyl, predominates over the pro-apoptotic metabolites of deprenyl, I-methamphetamine and I-amphetamine. Methamphetamine isn't formed if desmethyldeprenyl is administered directly. The administration could give desmethyldeprenyl a pharmacokinetic advantage over deprenyl. However, desmethyldeprenyl is still an FDA-unapproved substance. The possibility is that deprenyl may have unique anti-apoptotic effects.

The structural similarity to desmethyldeprenyl cannot be excluded at the present time. Use of available deprenyl as ophthalmic drops with or without oral intake with insulin ophthalmic drops can prevent the apoptosis of many of the healthy cellular components such as RPE, photoreceptors, and Bruch's membrane seen in age related macular degeneration and other oculopathies related to the retina.

### Example 30

Follow the instruction as described in the above EXAMPLE 1. There are patients with age related macular degeneration associated with cystoid macular edema. Treatment of this condition is an important part where the treatment of age related macular degeneration is to improve the acuity and closer vision. The treatment involves the Intravitreal injection of 4 mg (0.1 ml) triamcinolone acetonide to treat macular edema. The visual and anatomic responses were observed where there were complications related to the injection procedure and the corticosteroid medication. These patients' eye conditions were treated with 250 mg of oral acetazolamide twice daily for a month or so.

Our invention involves using intravitreal injection of triamcinolone acetonide with 1 or 2 units of insulin added to the injectate for its rapid uptake and augmentation-amplification effects of the therapeutic agent corticosteroid. It will make up 0.2 ml injectate which the injectate can be safely injected. The insulin will enhance the uptake of this corticosteroid, and will enhance the corticosteroid activity relieving the macular edema at the same time which this activity helps to reduce the ROS causing the damage to the photoreceptors and stabilize the membrane integrity. The use of insulin ophthalmic drops with corticosteroid two to three times a day as part of the protocol for treating age related macular degeneration and macular edema with acetazolamide.

### Example 31

Follow the instruction as described in the above EXAMPLE 1. Superoxide dismutases, catalases, lactoperoxidases, glutathione peroxidases and peroxiredoxins, small molecule antioxidants like ascorbic acid (vitamin C), tocopherol (vitamin E), uric acid, polyphenol antioxidants, and glutathione play important roles as cellular antioxidants by facilitating the removal or ROS. The most important plasma antioxidant in humans is probably uric acid. We have used uric acid in many ophthalmic conditions and to treat CVD for 30 years. Most of the above antioxidants can be incorporated to ophthalmic drops with insulin. The use of uric acid to prevent and to treat many oculopathies including age related macular degeneration.

### Example 32

Follow the instruction as described in the above EXAMPLE 1. Insulin composition with sodium fluorescein (and other dyes combination) is used for diagnosing the eye ball as well as retinal health and the disease of the eyes' blood supply. The blood supply plays a role in age related macular degeneration and diabetic retinopathy. Insulin will enhance the uptake and the circulation of the eye which the fluorescein will mark the afflicted tissue particularly in the blood vessels and the endothelial cells of the retina. The blood vessels are important for the health of the photoreceptors and the diagnosing underlying patho-physiology related to BV such as in diabetic retinopathy.

This diagnostic method called the “fluorangiography” is performed by means of the intravenous injection of a fluorescent substance with the following photography of the retina at different times. Apply ophthalmic insulin drops to both the eyes 30 minutes before the IV injection of fluorescent substance. Insulin can be injected up to 3 units with a fluorescent substance in addition. The fluorescent substance in blood arrives at the retina. The fluorescein colors the BV. This renders the BV visible due to the local effect of the insulin. The results will reveal the functional and pathophysiological state of the BV walls. Our invention of insulin ophthalmic solutions can be used to enhance the uptake of radioactive material used to diagnose eye diseases and/or used to treat all eye diseases (ARMD and Ophthalmic tumors).

### Example 33

Follow the instruction as described in the above EXAMPLE 1. Use of Chelation therapeutic agents with insulin: It is a known fact that the photoreceptors in retinitis pigmentosa and age related macular degeneration are undergoing changes and apoptosis due to deposits of fat, calcium, protenacious, and dysfunctional cellular complexes. These changes may take place in the choroid, RPE, Bruch's membrane, photoreceptors, and Muller cells.

It is likely that they do have many metallic and organic deposits like the lipoprotenacious material, iron, calcium, aluminum, and other metals in them causing death and due to death of cells and protenacious deposits. Chelation therapy locally or systemically with Ethylenediaminetetraacetic acid (EDTA), Methylsulfonylmethane (MSM), Alagebrium, and Deferoxamine (also known as desferrioxamine B, desferoxamine B, DFO-B, DFOA, DFB or desferal) will clear these clogged cell layers and photoreceptors cells undergoing changes due to metal pathology and lipoprotein complex. They remove any excess iron, calcium, and other metals as well as the fatty protenacious deposits which these may interfere with their physiological role resulting in pathological process leading to retinitis pigmentosa and ARMD.

It is known that the EDTA (Ethylenediaminetetraacetic acid) unclogs blood vessels and controls free radical damage due to lipid peroxidation by serving as a powerful antioxidant. It increases tissue flexibility by uncoupling age-related cross-linkages that are responsible for loss of cellular function and removes lead, cadmium, aluminum, and other metals.

This function restores enzyme systems to their proper functions, enhances the integrity of cellular, and mitochondrial membranes, and reduces the tendency of platelets to cause coagulation too readily which the platelets can clog the transportation system which unclogs the clogged draining vascular system. It increases tissue flexibility by uncoupling age-related cross-linkages (age-related glycation) which this function is responsible for the proper function of the glands. Millions of Americans have undergone Chelation therapy including the present inventor, to eliminate the arteriosclerotic vascular diseases and to reduce the metalloproteinase's with good results.

The inventor has used Chelation therapy with insulin with mild hyperthermia with wonderful results in ASVD. The present inventor has attempted to use EECP—Enhanced External Counter pulsation to treat cancers with IPT, use it with Chelation to clear BV including coronaries, and use therapeutic agents driven to the coronaries which will clear the coronary arteries to save the heart from MI and angina and prevent the CABG surgery and the use of expensive repeated coronary stents. The use of EDTA along with insulin as described in our invention can slow down, arrest, or reverse the changes in the choroidal capillaries, RPE and reduce the cataract development. This brings about the physiological status to the afflicted Age related macular degeneration.

### Example 34

Follow the instruction as described in the above EXAMPLE 1. Methylsulfonylmethane (MSM), is an organosulfur compound with the formula (CH₃)₂SO₂; a metabolite of DMSO. It is also known by several other names: DMSO2, MSM, Methylsulfonylmethane, methyl sulfone, and dimethyl sulfone. MSM is a supplement form of sulfur that is found in our living tissues. MSM supports healthy connective tissues like tendons, ligaments, muscle, and nervous tissue function including retina. MSM makes cell walls permeable, allowing water and nutrients to freely flow into cells, which the permeability allows the wastes and the toxins to properly flow from the retina, where the outflow is needed in the photoreceptors in age related macular degeneration.

MSM is an anti-oxidant in which MSM helps to clean the blood-stream. The MSM flushes toxins trapped in our cells including the photoreceptors, RPE, Bruch's membrane, and neovascularization of the choriocapillaries. The MSM is a foreign protein and free radical scavenger which the foreign protein is needed to maintain the photoreceptors function affected in age related macular degeneration. The body uses MSM along with Vitamin C to create new, healthy cells by preventing ROS damage and cleaning the toxins from ophthalmic structures. The MSM provides the flexible bond between the cells. We have prescribed MSM ophthalmic drops to many aged, Lyme disease, and cancer patients, which the patients reported, that their vision had improved.

MSM is soluble in water where it is a good solvent like DMSO. We have used aqueous solutions of MSM filtered, sterilized, and mixed with insulin. We used as eye drops to treat age related macular degeneration, retinitis pigmentosa, cataract, dry eye syndrome, glaucoma, and other oculopathies with good results. The use of MSM with insulin as eye drops can prevent (act as prophylactic in those who are genetically disposed), delay the onset, curtail, or cure the age related macular degeneration conditions.

We prepare the following eye drops containing: 1. EDTA, 2. Deferoxamine, 3. MSM, with added preservatives, antibacterial, and DMSO combined with insulin in proper concentrations. Any one of the chelating agents or combination of them can be used to formulate the eye drops. These eye drops are used before or after insulin drops as prophylactic and therapeutic agents for age related macular degeneration and other oculopathies.

### Example 35

U.S. Patent Application Publication Number: 2004/0054130 AI invention relates to compounds that have the ability to potentiate the physiological activity of insulin particular to the small peptide compounds or peptidomimetic compounds, where the compounds has the ability to potentiate one or more of the physiological activities of insulin. The peptides comprises of basic amino acids like lysine, arginine, homolysine, homoarginine or ornithine, neutral aliphatic amino acid, in either the L- or the D-form, such as glycine, leucine, alanine, phenylalanine or isoleucine, homo leucine, norleucine, homonorieucine, cyclohexylalanine, or homocyclohexylalanine and an aromatic amino acid, such as phenylalanine or tyrosine. The amino acids or amino acid analogues have a side chain having or delocalized electrons. These therapeutic agents can be added to the ophthalmic preparations of the insulin to enhance the insulin absorption and the activity to treat Age related macular degeneration and other oculopathies.

### Example 36

Follow the instruction as described in the above EXAMPLE 1. If the corneal, conjunctival and retinal BV are suspected of involved in oculopathies; they need to be tested using fluorescein as one of the method testing before treating Age related macular degeneration. The fluorangiography is performed by means of the intravenous injection of a fluorescent substance with following photography of the retina and the retina's BV at different times. The fluorescent substance in blood arrives at the retina which the substance colors the arteries, the capillaries, and the veins. The fluorescent substance renders BV visible, with the functional state of their walls. Use of our invention with insulin before the procedure or with IV injection of the dye demarks the afflicted blood vessels even better. Any thinning of the retinal blood vessels and associated ocular pathology is revealed by this method. Local use of these fluorescent substances to diagnose corneal and conjunctival pathology can be facilitated using a mixture of the dye and insulin or using ophthalmic insulin drops before instilling the marker dyes.

### Example 37

Follow the instruction as described in the above EXAMPLE 1.

Studies in a cross-sectional survey of men and women show that the use of statins (to reduce blood cholesterol levels) is associated with an 11-fold reduction in risk of macular degeneration. Statins are inhibitors of 3-hydroxy-3-methylglutaryl coenzyme A, i.e. HMG-CoA reductase inhibitors.

U.S. Patent Application Publication Number: 2003/0065020 AI describes a method of treating or preventing macular degeneration in patients by administering HMG-CoA reductase inhibitors. This invention discloses the treatment with HMG-CoA reductase inhibitors results in: (i) reduced accumulation of basal linear deposit in Bruch's membrane; (ii) protection of the outer retina from oxidative damage; and (iii) inhibition of endothelial cell apoptosis. Oral intake HMG-CoA reductase inhibitors can be used to treat ARMD to prevent the oxidative damage, clear the linear fatty deposits in the Bruch's membrane so that it can actively participate in the RPE and photoreceptors physiological function, and prevent RPE-photoreceptors apoptosis seen in this condition with the formation of Drusen deposits.

They exert their therapeutic effect against ARMD (a) by lowering the level of LDL cholesterol in the patient; (b) increasing the level of HDL cholesterol in the patient; and (c) lowering the level of triglycerides in the neovascularization choriocapillaries. There are various FDA approved HMG-CoA reductase inhibitors in use. They are selected from the group consisting of: fluvastatin (Lescol), cerivastatin (Baycol), atorvastatin (Lipitor), simvastatin (Zocor), pravastatin (Pravachol), lovastatin (Mevacor) and rosuvastatin (ZD 4522). HMG-CoA reductase inhibitor was prepared with a pharmaceutically acceptable carrier to generate a pharmaceutical composition and administering the pharmaceutical composition to the patient. We have prepared suitable ophthalmic drops from one of these statins to be used with insulin ophthalmic drops. If it is not possible, use the statins orally with insulin drops to inhibit the pathological process ARMD.

We recommended the statins drugs in varying doses to almost all the patients with these conditions including diabetic retinopathy, cataract, retinitis pigmentosa, and other oculopathies and those with cholesterol level above 180 mg %. This method of therapy not only saves the eyes from various oculopathies including ARMD, it also saves the patients from the cardiovascular diseases.

### Example 38

Follow the instruction as described in the above EXAMPLE 1. U.S. Pat. No. 6,525,019 B2 discloses the therapeutic agent melanin for inhibition of angiogenesis of ARMD. Melanin located within specific cells called melanocytes. Melanins are present in the skin, hair and eyes where they impart the color, play a role in light absorption and acts as free-radical scavenger (antioxidant). Individuals with lighter iris color have been found to have a higher incidence of age-related macular degeneration (ARMD) than those with darker iris color; lighter eye color is coupled with an increased risk of ARMD progression (Frank R N, Puklin J E, Stock C, Canter L A (2000). “Race, iris color, and age-related macular degeneration”. Trans Am Ophthalmol Soc 98: 109-15; discussion 115-7).

Facts indicate that individuals with increased iris pigmentation have a decreased risk of developing ARMD. Given that the increased levels of eumelanin appear to be more protective than pheomelanin, and the light-absorbing characteristics of melanin are thought to be responsible for this protective effect (Hammond B R, Jr, Fuld K, Snodderly D M. Iris color and macular pigment optical density. Exp Eye Res. 1996, 62:293-297). An alternative hypothesis is that increased levels of melanin may protect against age-related increases in lipofuscin (implicated in photo-oxidative mechanisms). However, these prior studies do not teach, discuss, or suggest the antiangiogenic ability of melanin to inhibit blood vessel growth and macular degeneration, as disclosed in the invention U.S. Pat. No. 6,525,019 B2.

It will be appreciated that the term “melanin” as used herein means both soluble and insoluble forms of melanin, including eumelanin and pheomelanin, and precursors fragments of these molecules. The term “melanin-promoting compound” as used herein means any compound capable increasing the amount or activity of melanin in vivo. Examples of melanin-promoting compounds are tyrosinase, melanocytes stimulating hormone (MSH), melanocytes concentrating hormone (MCH), minocycline, latanoprost, melanotan-I, prostaglandins and compounds with prostaglandin activity, ACTH, melanocortin receptor antagonists, endothelin, rifabutin, diacycloglycerols, arbutin, amiodarone, pefloxcin, chlorpromazine, desipramine, sulfasalazine, zidovudine, clofazimine, bergapten, metenkephalin and cyclophosphamide. Such alternative compounds may modify the production or bioactivity of melanin. The above melanogenic therapeutic agents can be used as ophthalmic drops with insulin to increase the melanin, protect the RPE and retina from the ROS and inhibit the angiogenesis seen in ARMD.

### Example 39

Follow the instruction as described in the above EXAMPLE 1. U.S. Pat. No. 6,936,043 B2 and U.S. Pat. No. 6,942,655 B2, Bressler, disclose (Bressler N M. Photodynamic therapy of sub foveal choroidal neovascularization in age related macular degeneration with verteporfin: two year results of 2 randomized clinical trials—TAP report 2. Arch Ophthalmol 2001, 119:198-207. Photodynamic therapy of sub foveal choroidal neovascularization in age related macular degeneration with verteporfin: one year results of 2 randomized clinical trials—TAP report. Treatment of age related macular degeneration with photodynamic therapy (TAP) study group. Arch Ophthalmol 1999; 117:1329-45). Using photodynamic therapy (PDT) to treat ARMD may need many treatments which can further damage the retina.

PDT prevents or alters the function of the neovascular tissue by using low energy light to generate reactive species within the blood vessels, or within and around the vessels, to thereby damage these vessels and prevent further growth. The use of insulin ophthalmic drops will increase the concentration of photosensitizing agents to be delivered to the fovea centralis and macula lutea, the site of the age related macular degeneration vasculogenesis which then can be photocoagulated with focused laser or other effective lights.

### Example 40

Follow the instruction as described in the above EXAMPLE 1. Intavitreous bevacizumab 1.25 mg injections has been given as treatment is associated with a greater chance of moderate vision recovery and a reduced risk of moderate vision loss and improves mean visual acuity at one year in patients with neovascular ARMS compared with standard treatment. In addition, more than 45% of the patients treated with bevacizumab improved 10 or more letters, a threshold that exceeds the variability of the measurement of visual acuity and represents the proportion of patients recovering vision with least complications. (Spaide R F, Laud K, Fine H F, Klancnik J M Jr, Meyerle C B, Yannuzzi L A, et al. Intravitreal bevacizumab treatment of choroidal neovascularization secondary to age related macular degeneration. Retina 2006, 26:383-90. Adnan Tufail et al. BMJ 2010; 340:c2459 doi: 10.1136/bmj.c2459 (Published 10 Jun. 2010) Cite this as: BMJ 2010; 340:c2459; Bevacizumab for neovascular age related macular degeneration (ABC Trial): multicentre randomized double masked study).

Addition of insulin 1 to 2 IU of insulin to the intravitreal injectate of Bevacizumab will augment and amplify the effects of this MAB in curing, curtailing, improving the vision, or preventing the progression of ARMD. Bevacizumab can be formulated with insulin to be administered topically in the conjunctival sac instead of intravitreal injection.

### Example 41

Follow the instruction as described in the above EXAMPLE 1. U.S. PATENT APPLICATION PUB. NO: 2005/0239757 A1 disclose methods for treating ARMD and other degenerative ocular condition using progesterone. The hormone may be administered through routes include ocular, sub lingual, intradermal injection, subcutaneous injection, intravenous injection, intranasal, transdermal, trans conjunctival, or aerosol mist through any orifice or through the skin. The present invention relates to ameliorating, treating, and/or preventing macular degeneration and/or any degenerative ocular condition, disorder, or disease (collectively “condition”), using dilute hormone dilutions is provided.

Observations that lead to and are a part of the present disclosure, may suggest the possibility of an allergic reaction to the steroid hormone progesterone as a possible cause of macular degeneration and other disorders. This treatment for ARMD involves desensitizing a body's response to its own innate hormones.

### Example 42

Follow the instruction as described in the above EXAMPLE 1. U.S. PATENT APPLICATION PUB. NO.: 2004/0180090 AI discloses methods and compositions for the treatment of macular degeneration by administering a combination of polyvinyl pyrollidone (PVP), procaine and thiamine to a mammalian host. The first group includes macromolecular compounds that may be selected from the following: a) polyvinyl pyrollidone (available as Kollidon™; from BASF, or Plasdone C from GAF Cooration); b) pneumococcal polysaccharides or c) lipopolysaccharides (group 1);

The second group includes the salts of lidocaine, chloroprocaine, tetracaine, procaine or piperocaine (group 2); The third group includes the salts thiamine, riboflavine, papaverine, papaveraldine, paveril, D-biotin or D-biotin in esterified or salt form (group 3); The fourth group includes insulin or zymosan (group 4). Further, details concerning various components of the present invention may be found in U.S. Pat. No. 4,618,490, incorporated herein by reference.

### Example 43

Follow the instruction as described in the above EXAMPLE 1. U.S. Pat. No. 4,656,188 discloses the angiotensin converting enzyme inhibitors (ACE inhibitors) are useful in the treatment of senile macular degeneration. Their discovery is based that the senile macular degeneration is a poorly characterized disease state of the elderly which appears to result from a poor blood supply to the macular region of the eye. As a result, vision is lost in the central region of the eye while partial peripheral vision is retained. The disease progresses with increased vision loss, one eye at a time.

Experience with ACE inhibitors as antihypertensive agents has shown a tendency for them to accumulate in the eye resulting in unexpectedly high concentrations in ocular tissue [Igic et al., Exp. Eye Res. 30, 299 (1980)]. These high concentrations result in selective ocular vasodilatation thereby increasing local blood flow to the otherwise ischemic tissue thus preventing damage to the eye. The angiotensin converting enzyme inhibitor useful as the active ingredient in the novel method of treatment and pharmaceutical formulations of this invention is selected from: enalapril, enalaprilat, lisinopril, captopril, ranipril, perindopril, zofenopril, quinapril, pentopril, cilazapril, pivopril, fosenopril, indolapd, indalapril, phenacein, fentiapril, alacepril, perinodopril, mugenic acid, ancovenin, CI-925, CGS 14824% CGS 14831, WY 44221, CI-928, SQ 28853, SQ 27786, CGS16617, MC 838, K 26.

### Example 44

Follow the instruction as described in the above EXAMPLE 1. U.S. PATENT APPLICATION PUB. NO.: 200710160592 A1 inventions provides a method for treating macular degeneration utilizing a therapeutic agent delivery system that is disposed in proximity of the sclera which one or more therapeutic agents are injected or diffused into the sclera to provide for the dissolution of accumulated metabolic waste products in Bruch's membrane. The objects of the present invention are achieved by apparatus and method for delivering a natural enzyme lipase (lipoprotein lipase) into the posterior sclera in close proximity to the macula that will dissolve lipid deposits in the body of the membrane and assist in their removal through the choroidal circulation.

### Example 45

Follow the instruction as described in the above EXAMPLE 1. U.S. PATENT APPLICATION PUB. NO.: 200710037782 A1 disclose the therapeutic agent for aging macular degeneration comprises a progesterone derivative with special formulation. The progesterone derivative represented by the formula their special formula is described in W095126974. It is known to have an inhibitory action on neovascularization and is useful as a therapeutic agent for malignant tumor, diabetic retinopathy, rheumatism and the like.

### Example 46

Follow the instruction as described in the above EXAMPLE 1. U.S. PATENT APPLICATION PUB. NO.: 2007/0027102 AI discloses methods of treating all forms of wet, age related macular degeneration by administration of an anti-vascular endothelial growth factor (anti-VEGF) compound. Research of wet ARMD shows that vascular endothelial growth factor (“VEGF”) is one of the major factors causing both abnormal blood vessel growth (angiogenesis) and blood vessel leakage in the eye. Substantial peer-reviewed research has found high concentrations of VEGF in the eyes of humans afflicted with wet ARMD. For example, in a study published by the New England journal of Medicine, vitreous levels of VEGF were shown to be very high in patients with angiogenic diseases, but were negligible in patients undergoing the same type of surgery for non angiogenic diseases. (Aiello et al., 33, Feb. 1, 2007, the ocular VEGF levels are elevated in patients with active Diabetic Macula Edema (DMES). New. Eng. J. Med. 1480-87 (1994). A. Vinores et al., Upregulation of vascular endothelial growth factor in ischemic and nonischemic human and experimental retinal disease, 12(1) Histol. Histopathol. 99-109 (1997. Sato T N, Tozawa Y, Deutsch U, Wolburg-Buchholz K, Fujiwara Y, Gendron-Maguire M, Gridley T, Wolburg H, Risau W, Qin Y. 1995. Distinct roles of the receptor tyrosine kinases Tie-1 and Tie-2 in blood vessel formation. Nature 376:70-74. Kwak N, Okamoto N, Wood J M, Campochiaro P A. 2000. VEGF is an important stimulator in a model of choroidal neovascularization. Invest Ophthalmol V is Sci (in press)).

The anti-VEGF agents may be, for example, VEGF antibodies or antibody fragments, such as those described in U.S. Pat. Nos. 6,100,071; 5,730,977; and WO 98145331. The anti-VEGF agents can also be administered topically, by patch or by direct application to the eye, or by iontophoresis or intravitreal administration of the anti-VEGF. Anti-VEGF is aptamer administered topically into the eye. Aptamers are oligonucleic acid or peptide molecules that bind to a specific target molecule. Natural aptamers exist in riboswitches.

Nucleic acid aptamers are nucleic acid species that have been engineered through repeated rounds of in vitro selection or equivalently, SELEX (systematic evolution of ligands by exponential enrichment) to bind to various molecular targets like small molecules, proteins, nucleic acids, and even cells, tissues, and organisms.

Scientists know that unless a tumor connects to a supply of blood, it will grow to a mere 1,000 cells and then stop. The agents being evaluated target various biological functions involved in angiogenesis, including vascular endothelial growth factor endothelial cell proliferation (thalidomide, IFN-α), and matrix metalloproteinase's (marimastat). Many of the anti-angiogenesis drugs used today attack the VEGF pathway in cancers. The Bevacizumab (Avastin®) a monoclonal antibody a man-made version of an immune system protein—that binds to VEGF and keeps it from reaching the VEGF receptor and there are many other being tested for ARMD to prevent the choroidal neovascularization (angiogenesis) as described in the following publications.

The agents being evaluated target various biological functions involved in angiogenesis, including vascular endothelial growth factor (bevacizumab), various studies of different MAB have shown the efficacy of this therapeutic agents in curtailing the advancement of choroidal neovascularization (angiogenesis). These studies are made by injecting the MAB intravitreally (Gragoudas E S, Adamis A P, Cunningham E T Jr, Feinsod M, Guyer D R. Pegaptanib for neovascular age related macular degeneration. N Engl J Med 2004, 351:2805-16. Rosenfeld P J, Moshfeghi A A, Puliafito C A. Optical coherence tomography findings after an intravitreal injection of bevacizumab (avastin) for neovascular age-related macular degeneration. Ophthalmic Surg Lasers Imaging 2005.36:331-5. Rosenfeld P J, Brown D M, Heier J S, Boyer D S, Kaiser P K, Chung C Y, et al. Ranibizumab for neovascular age related macular degeneration. N Engl J Med 2006, 355:1419-31. Ziemssen F, Grisanti S, Bartz-Schmidt K U, Spitzer M S. Off-label use of bevacizumab for the treatment of age related macular degeneration: what is the evidence? Drugs Aging 2009, 26:295-320. Patel P J, Bunce C, Tufail A. A randomised, double-masked phase III/IV study of the efficacy and safety of avastin(R) (bevacizumab) intravitreal injections compared to standard therapy in subjects with choroidal neo vascularization secondary to age related macular degeneration: clinical trial design. Trials 2008, 9:56. Lazic R, Gabric N. Intravitreally administered bevacizumab (Avastin) in minimally classic and occult choroidal neovascularization secondary to age related macular degeneration. Graefes Arch Clin Exp Ophthalmol 2007, 245:68-73.)

Other drugs, like sunitinib (Sutent®) and sorafenib (Nexavar), are small molecules that attach to the VEGF receptor. This keeps it from being turned on and making new blood vessels. Some drugs already used to treat cancer have been found to affect blood vessel growth, too. Some other drugs used to treat cancer, such as thalidomide (Thalomid®) and lenalidomide (Revlimid®), are known to affect blood vessel growth. These drugs have never been used for treatment of ARMD. We plan to use these medications as ophthalmic drops with Insulin in our invention to treat ARMD and other diseases of the eye.

Cytokines are proteins that are produced by cells which interact with immune system cells in order to regulate the body's response to disease and infection. Cytokines are diverse; they locate target immune cells and interact with receptors on the target immune cells by binding to them. The interaction triggers or stimulates specific responses by the target cells. Overproduction production of certain cytokines by the body can result in disease. For example, it has been found that interleukin-1 (IL-1) and tumor necrosis factor-alpha (TNF-alpha) are produced in excess in rheumatoid arthritis and many autoimmune diseases where they are involved in inflammation and tissue destruction. ARMD pathogenesis is said to involve a TNF-mediated inflammatory or degenerative processes.

TNF is a biologically occurring cytokine present in humans and other mammals. It plays an important role in the immune response and the inflammatory response to infection. It is formed by the cleavage of a precursor transmembrane protein, forming soluble molecules which aggregate in vivo to form trimolecular complexes. These complexes afterward bind to receptors found on a variety of cells. Binding produces an array of pro-inflammatory effects, including release of other pro-inflammatory cytokines, including IL-6, IL-8, and IL-1; release of matrix metalloproteinases; and up regulation of the expression of endothelial adhesion molecules, further amplifying the inflammatory and immune cascade by attracting leukocytes into extra vascular tissues as seen in choroidal neovacularisation tissue in ARMD. That is why MAB are effective in treatment of ARMD with insulin.

Antibodies (immunoglobulins) are proteins produced by B lymphocytes in response to specific exogenous foreign antigen molecules. Monoclonal antibodies (MAB), identical immunoglobulin copies, and they are fusion proteins which recognize a single antigen, are derived from clones (identical copies) of a single set of B cells. This technology has enabled huge quantities of an immunoglobulin with a specific target to be mass produced. MAB with a high affinity or attraction for a specific cytokine will have a propensity to reduce the biologic activity of that cytokine. Substances which reduce the biologic effect of a cytokine can be a blocker, inhibitor, and antagonist to cytokines. Biologic drugs have been developed to inhibit IL-1 or TNF-alpha that works by inhibiting or preventing these cytokines binding to its cell surface receptors. TNF-alpha inhibitors commonly available are Enbrel (etanercept), Remicade (infliximab), and Humira (adalimumab) are TNF blockers and many others anti-cytokine therapies are under development.

Age Related Macular Degeneration of both “wet” and “dry” macular degeneration; implicate excess TNF and/or the participation of TNF-mediated inflammatory or degenerative path in their pathogenesis. Treatment of patients with these disorders through the conjunctival sac delivered leads to prevention, delay progression and lead to visual improvement of ARMD. Etanercept, golimumab, or certolizumab pegol may be administered concurrently with memantine (delivered orally) to further reduce ARMD related pathology with or without optic nerve damage. Also soluble TNF receptor type 1 and pegylated soluble TNF receptor type 1 may be administered. Pegapanib, ranibizumab, and bevacizumab (Avastm™, Genentech), a recombinant humanized monoclonal IgGI antibody that inhibits the biologic activity of human vascular endothelial growth factor (VEGF), may also be administered both the treatment or prevention of Macular degeneration and/or neovascularization and thereby produce visual improvement, prevent or delay of impending visual loss. In addition these disorders are known to involve IL-I. Therefore treatment of these disorders with an IL-I antagonist, such as IL-I RA (Kineret) or IL-I Trap administered by effective dose of the IL-I antagonist reaches Choroidal—Retinal vascular system and thenceforth the retina, delivered utilizing a prolonged treatment schedule. Administering these MAB through the vertebral venous system is unpredictable, requiring large doses of medications making it expensive, and associated systemic effects and the therapeutic agents may not reach in enough therapeutic concentrations to be effective against the ARMD and other oculopathies.

Our invention of Conjunctival sac administration therapeutic agents MAB with Insulin involves anatomically localized delivery performed so as to place the therapeutic molecule directly in the vicinity of the pathologically afflicted site i.e. ARMD. Conjunctival sac administration of therapeutic agents is not limited to, the following types of administration: subconjunctival, parenteral; subcutaneous; intramuscular; intravitreal, retro bulbar (behind the eye ball), subarachnoid space, intranasal, epidural and intra arachnoid subdural spaces. Topical Localized administration of MAB with insulin for the treatment of localized clinical disorders such as ARMD and Retinitis pigmentosa has various clinical advantages over the use of standard systemic treatment. Locally administered therapeutic agents with insulin of this invention distributes through local capillary, venous, arterial, and lymphatic routes to reach the ARMD therapeutic target. Etanercept being a potent anti-inflammatory agent also has significant and vital anti-apoptotic effects, which may be of particular importance in treating retinal neurodegenerative diseases such as ARMD and retinitis pigmentosa which are associated with destruction of photoreceptors such as Cones in the Macula and rods in the rest of the retina, where apoptosis plays a pathogenetic role. MAB combined with insulin, can prevent and protect the photoreceptors from further damage and apoptosis.

### Example 47

Follow the instruction as described in the above EXAMPLE 1. U.S. PATENT APPLICATION PUB. NO.: 200910155381 A1 determine the susceptibility to ARMD, then use medication comprising lutein (wherein the carotenoid is lutein and/or zeaxanthin) and/or zeaxanthin and/or certain antioxidants (or a mixture thereof) is tailored to that individual by providing an effective amount of a carotenoid and/or vitamin C, vitamin E; beta carotene, zinc and/or copper, and/or a mixture there of (the AREDS Cocktail) to said subject. All the natural therapies can be combined with insulin ophthalmic drops.

### Example 48

Follow the instruction as described in the above EXAMPLE 1. U.S. Pat. No. 6,949,518 B1 discloses a method for treating macular degeneration and/or treating optic nerve degeneration of a patient comprises administering topiramate with a dosage pharmaceutically effective to suppress degeneration or induce growth of new optic nerve fibers over a sustained period. The topiramate compound can be combined with one or more IOP-lowering agents administered topically to treat glaucoma in addition.

### Example 49

Follow the instruction as described in the above EXAMPLE 1. U.S. PATENT APPLICATION PUB. NO.: US 2007J0010746 A1 and U.S. Pat. No. 7,351,193 B2 disclose treatment of age related macular degeneration treated by radiation delivered from a miniature x-ray tube inserted via a catheter around the globe of the eye, to a position behind the macula. These patents disclose the x-ray treatment enhancement using a radio sensitizing drug, and can be combined with PDT. Our invention provides a method to remove free radicals after the x-ray exposure of the eye and preserve the sensitive retina from after effects of radiation

### Example 50

Follow the instruction as described in the above EXAMPLE 1. U.S. Pat. No. 5,314,909 discloses the topical application of non-steroidal anti inflammatory agents (NSAID) to treat ARMD. There is a well documented effect of Indomethacin in the treatment of cystoid macular edema, a condition. Senile macular degeneration has an increased permeability of the retinal capillaries and some destruction of retinal pigment epithelium. They disclose the use of indomethacin, diclofenac, ketorolac, flurbiprofen, and the like to treat this condition.

### Example 51

Follow the instruction as described in the above EXAMPLE 1. U.S. Pat. No. 7,381,404 B2 discloses the treating of ARMD by administering a caged detergent to the individual; and selectively applying two-photon irradiation to the caged detergent in Bruch's membrane to activate the detergent, resulting in an increase in diffusion across the membrane wherein the detergent is further defined as being caged by a compound comprising at least one o-nitrobenzyl, desyl, phenacyl, trans-o-cinnamoyl, coumarinyl, quinoline-2-onyl, xanthenyl, thioxanthenyl, selenoxanthenyl, anthracenyl, or stilbenzyl group.

### Example 52

Follow the instruction as described in the above EXAMPLE 1. Various patents disclose the method of administering photosensitive compounds which are activated by various physical methods such as: U.S. Pat. No. 5,756,541 discloses administering a photoactive compound; U.S. Pat. No. 5,798,349 describes treating ARMD by administering a liposomal formulation of a green porphyrin; U.S. Pat. No. 5,935,942 describes co-administering intravenously a fluorescent dye encapsulated with heat sensitive liposomes and a tissue-reactive agent activated by Irradiation; U.S. Pat. No. 6,140,314 discloses methods of co-administration of a tissue-specific factor effective to impair growth or regeneration of a blood vessel in wet ARMD

### Example 53

Follow the instruction as described in the above EXAMPLE 1. U.S. Pat. No. 6,046,223 discloses a method for treating and/or preventing macular edema and age related macular degeneration which comprises topical administration of carbonic anhydrase inhibitors to the eye such as Dorzolamide, acetazolamide, methazolamide, and other compounds which are described in U.S. Pat. Nos. 5,153,192; 5,300,499; 4,797,413; 4,386,098; 4,416,890 and 4,426,388. Studies of patients who respond to acetazolamide treatment typicality show epithelial cell dysfunction.

These cells, which line the innermost layer of the choroid—the RPE have villi-like projections which interdigitate with the retinal photoreceptors. This flexible intimate association between pigment epithelial cells and photoreceptors is of critical importance to retinal health. The photoreceptors are highly active metabolically and produce waste metabolites at a great rate. The pigment epithelial villi absorb catabolites from photoreceptor cells, facilitate the regeneration of photo pigment, and provide nutrients via their closely associated choriocapillaries vascular network passing through the Bruch's layer. Fluorescein angiographies of the pigment epithelium in individuals with macular edema shown to be responsive to acetazolamide demonstrate leakage of dye into the photoreceptor area. This leakage is inhibited by treatment with acetazolamide.

Macular degeneration is the most common cause of acquired legal blindness. Instead of fluid accumulating in the outer retina, hard accumulations of lipofuscin, a metabolic waste product, tend to accumulate between the photoreceptors and the villi of the retina pigment epithelium. These accumulations gradually enlarge, and in their early pathologic phase create discrete accumulations known as drusen.

The lipofuscin is believed to accumulate as a result of a process known as apoptosis, a breaking off of the photoreceptor elements. Shedding of the cellular components of the photoreceptors is constantly occurring in a healthy retina. Good retinal pigment epithelial metabolism generally insures a rapid clearance of such catabolic byproducts of vision. As drusen accumulate and begin to coalesce, vast areas of retinal photoreceptors become permanently disengaged from their neighboring retinal pigment epithelial villi leading to their pathologic change and apoptosis. These sections of retina affected become blind. The greatest tendency among the aging population is for drusen to accumulate in the actual central area of vision and the macula. Current therapy lacks any substantive clinical scientific basis with zinc in tablet form as one attempted method of treatment.

### Example 54

Follow the instruction as described in the above EXAMPLE 1. U.S. PATENT APPLICATION PUB. NO.: Us 2008/0 139592 AI discloses a method and composition of Carboxyamidotriazole Orotate (CM Orotate) for treating age related macular degeneration and other angiogenesis-dependent diseases. These inventors describe the use of antiangiogenesis methods to treat many angiogenesis diseases and treatment of ARMD as disclosed in U.S. Pat. No. 6,525,019 B2.

### Example 55

Follow the instruction as described in the above EXAMPLE 1. Dawson et al. describe that the Pigment epithelium derived factor is potent (PEDF) inhibitor of angiogenesis (Dawson D. W., Volpert O. V., Gillis P., Crawford S. E., Xu H., Benedict W., Bouck N. P. Pigment epithelium-derived factor: a potent inhibitor of angiogenesis. Science (Washington D.C.), 285: 245-248, 1999). Volpret et al. describe the anti angiogenic effect of Interleukin-4 (Volpert O. V., Fong T., Koch A. E., Peterson J. D., Waltenbaugh C., Tepper R. I., Bouck N. P. Inhibition of angiogenesis by interleukin 4. J. Exp. Med., 188: 1039-1046, 1998.). Thus the PEGF and interleukin-4 can be used in ARMD with our invention to prevent, curtail, or cure the condition.

### Example 56

Follow the instruction as described in the above EXAMPLE 1. Metmormin is extensively used in type II diabetics. The most popular brand-name combination was metformin with rosiglitazone, sold as Avandamet. Metformin increases the sensitivity to insulin, prevents uncontrolled cell division as seen in cancers and angiogenisis. It is aboth an antidiabetic and anticaner agent. Diabetics to have high incidence for ARMD and Metformin opthalmic preparation with insulin can bring more physiologic status to the photoreceptors, RPE and choriocapilarres, thus preventing ARMD and progression of ARMD. By increasing the sensitivity to insulin, the metformin can be very effective in treating the ARMD associated with diabetic retinopathy. In addition to suppressing hepatic glucose production, metformin increases insulin sensitivity, enhances peripheral glucose uptake, increases fatty acid oxidation. Studies show that the metformin to have bacteriostatic, antiviral, antimalarial, antipyretic and analgesic actions. (Quoted from Chemical Abstracts, v.45, 24828 (1951) Garcia E Y. Fluamine, a new synthetic analgesic and antiflu drug. Jouranl Philippine Med Assoc. 1950; 26:287-93). This study need to be explored further. Metformin has anticancer effect, in that it prevents the cell division. It has been shown to be effective in the treatment of endometriosis. In similar fashion, it can prevent the endothelial cell division in the formation of new BV seen in the ARMD. Mmetformin is a potent inhibitor of endometrial cancer cell proliferation, acting to arrest the cancer cells' reproductive cycle, inducing cell death through apoptosis, and decreasing gene expression of an enzyme complex called human telomerase reverse transcriptase (hTERT) that contributes to unregulated cell replication. Many of these effects were triggered by metformin's activation of the AMP protein kinase (AMPK) complex, and are identical to those induced by calorie restriction (Cantrell L A, Zhou C, Mendivil A, Malloy K M, Gehrig P A, Baejump V L. Metformin is a potent inhibitor of endometdal cancet' cell prolifemtion—implications for a novel treatment stmtegy. Gynecol Oncol. 2010 January; 116 (I):92-8). Based on these observations, other gynecological researchers have begun to use metformin as part of a “conservative” approach (using fewer, less-invasive procedures) to their management of endometrial hyperplasia and endometrial cancer (Stanosz S. An attempt at conservative treatment in selected cases of type I endometrial carcinoma (stage I a/G I) in young women. Eur] Gynaecol On col. 2009; 30 (4):365-9. Goepp Julius, About Metformin, Life Extnsion, November 2010, pages 41-51). Addition of vitamin K with or without metformin can also be use in treating ARMD. Unique mechanism how vitamin K works is demonstrated recently in bile duct cancers and leukemia autophagy. Here the cancer cells essentially “eat” themselves by releasing their own digestive enzymes internally. By still another unique mechanism, vitamins C and K in combination contribute to cancer cell death by autoschizis, whereby cells simply split open, spilling their contents (Verrax J, Cadrobbi J, Delvaux M, et al. The association of vitamins C and K3 kills cancer cells mainly by autoschizis, a novel form of cell death. Basis for their potential use as coadjuvants in anticancer therapy. Eur J Med Che. 2003, May; 38 (5):451-7.21). Finally, three of vitamin K′ s synergistic anticancer mechanisms have recently been identified. Vitamin K3 inhibits DNA-building enzymes, Vitamins K2 and K3 block new blood vessel formation essential to support: the rapid growth of tumor tissue which can take place in neovascularization in ARMD. (Matsubara K, Kayashima T, Mori M, Yoshida H, Mizushina y. InhibitOl-Y effects of vitamin K3 on DNA polymerase and angiogenesis. Jnt J Mol Med. 2008 September; 22(3):38 1-7). Vitamin K3 disrupts crucial intracellular communications networks composed of microtubules, preventing the cells from proliferating in a coordinated fashion (Yoshiji H, Kuriyama S, Noguchi R, et al. Combination of vitamin K2 and the angiotensin-converting enzyme inhibitor, perindopril, attenuates the liver enzyme-altered preneoplastic lesions in rats via angiogenesis suppression (J Hepatol. 2005 May; 42(5):687-93. Acharya B R, Choudhul-Y 0, Das A, Chakrabm-ti G. Vitamin K3 disrupts the microtubule networks by binding to tubulin: a novel mechanism of its anti proliferative activity. Biochemistry. 2009 Jul. 28; 48(29):6963-74. Felix Difara, remarkable anticancer properties of Vitamin K. Life Extnsion, November 2010, pages 67-72). Opthalmic preparation of Metformin and/or vitamin K3 can be used with Insulin for the treatment of ARMD to prevent angiogenisis and maintain normal physiologic state.

Numerous modifications; alternative arrangements of steps explained and examples given herein may be devised by those skilled in the art without departing from the spirit and the scope of the present invention. The appended claims are intended to cover such modifications and arrangements. Thus, the present invention has been described above with particularity and detail in connection. This is presently deemed to be the most practical and preferred embodiments of the invention. The invention will be apparent to those of ordinary skill in the art that numerous modifications, including, but not limited to, variations in size, materials, shape, form function, and manner of procedure, assembly, and the use may be made.

The preferred embodiment of the present invention has been described. The invention should be understood that various changes, adaptations, and modifications may be made thereto. It should be understood, therefore, that the invention is not limited to details of the illustrated invention. This method can be used to diagnose and treat all the oculopathies as well as prevent them.

Although the instant invention has been described in relation to particular embodiments thereof, many other variations and modifications and other uses will become apparent to those skilled in the art.

What is claimed is:

1. A method of treating age related macular degeneration comprising the step of topically instilling a therapeutically effective dose of insulin to an age related macular degeneration afflicted eye's conjunctival sac in humans and animals to be delivered to the fovea centralis and macula lutea; the site of the age related macular degeneration.

2. The method of treating age related macular degeneration according to claim 1 further comprising the step of instilling an additional medicine selected from a group comprising therapeutic, pharmaceutical, biochemical, nurticeuticals, biological agents, biological compounds, organic agents, and inorganic agents to said afflicted eye.

3. A method of treating age related macular degeneration comprising the step of topically instilling a therapeutically effective dose of IGF-1 to an age related macular degeneration afflicted eye conjunctival sac to be delivered to the fovea centralis and macula lutea, the site of the age related macular degeneration.

4. A method of treating age related macular degeneration comprising the step of topically instilling a therapeutically effective dose of insulin and IGF-1 in combination to the afflicted eye conjunctival sac to be delivered to the fovea centralis and macula lutea, the site of the age related macular degeneration.

5. The method of treating age related macular degeneration according to claim 3 further comprising the step of applying at least one other application selected from a group comprising a therapeutic agent, pharmaceutical, biochemical, nurticeutical, biological agent and biological compound to said afflicted.

6. The method of treating age related macular degeneration according to claim 2 wherein said therapeutic agent is selected from a group comprising cyclosporins in a base.

7. The method of treating age related macular degeneration according to claim 2 wherein said therapeutic agent is selected from a group comprising Monoclonal Antibodies Remicade™, Etanercept, Embrel™, and Humira™, TNF anti TNF agents, agents targeting TNF-α and B cells (anti-CD20, anti-CD22).

8. The method of treating age related macular degeneration according to claim 2 wherein said therapeutic agent is selected from a group comprising testosterone; DHEA, estrogens; Hydroxychloroquine (Plaquenil) and azathioprine (Imuran).

9. The method of treating age related macular degeneration according to claim 2 wherein said known therapeutic agents are ophthalmic preparations selected from a group comprising Anetholdithiolthione (ADT, 5-[p-methoxyphenyl]-3H-1,2-dithiol-3-thione), hyaluronic acid, Diquafosol (INS365 Ophthalmic) and Rebamipide.

10. The method of treating age related macular degeneration according to claim 2 wherein said therapeutic agent is a combination of two or more agents selected from a group comprising cyclosporins, estrogens, DHEA, and testosterone in combination or as separate therapeutic agents.

11. The method of treating age related macular degeneration according to claim 2 wherein said therapeutic agent is a chelating agent selected from a group comprising Methylsulfonylmethane (MSM), Ethylenediaminetetraacetic acid (EDTA), Alagebrium and Deferoxamine.

12. The method of treating age related macular degeneration according to claim 2 further comprising the step of using an uptake facilitator to further enhance a therapeutic effect selected from a group comprising electroporation, iontophoresis, Vibration methods, sonophoresis, vibroacoustic, vibration, physical heat, magnetic field, radio frequency field, microwave, and laser light.

13. The method of treating age related macular degeneration according to claim 2 wherein said therapeutic agents are selected from a group comprising antibiotics, analgesics, NSAIDs and antivirals.

14. The method of treating age related macular degeneration according to claim 2 wherein said therapeutic agents are selected from a group comprising Gamma linolenic acids, omega 3 fatty acids (DHA and EPA), vitamins A, B₆, C, E, zinc, selenium, taurine, lutein, azaxanthins, Resveratol, Proanthocyanidins curcumin, bioblavanoids, liposome-based; retinoids; glycerin, propylene glycol, glutathione, uric acid, polyphenol antioxidants, superoxide dismutases, catalases, lactoperoxidases, glutathione, zeaxanthin peroxidases, peroxiredoxins, and calcium ion ophthalmic drops compositions

15. The method of treating age related macular degeneration according to claim 2 that said therapeutic agents are selected from a group comprising levocabastine (Livostin); antihistamines (antolozine, Pheniramine maleate), vasoconstrictors (naphazoline hydrochloride, phenylephrine); Naphazoline hydrochloride, sodium cromoglycate, Naphcon A, non-steroidal anti-inflammatory drugs (NSAID); Ketorolac trimethamine; and corticosteroids (hydrocortisone, Dexamethasone, prednisolone).

16. The method of treating age related macular degeneration according to claim 2 wherein said therapeutic agents are acetazolamide, and Brinzolamide.

17. The method of treating age related macular degeneration according to claim 2 wherein said therapeutic agents is selected to have the ability to potentiate at least one of the physiological activities of insulin that the peptide comprises a basic amino acid lysine, arginine, homolysine, homoarginine or ornithine; L- or D-form of neutral aliphatic amino acid, glycine, leucine, alanine, phenylalanine or isoleucine, homo leucine, norleucine, homonorieucine, cyclohexylalanine, or homocyclohexylalanine; an aromatic amino acid, phenylalanine or tyrosine.

18. The method of treating age related macular degeneration according to claim 2 wherein said therapeutic agents are a HMG-CoA reductase inhibitor selected from a group comprising fluvastatin (Lescol), cerivastatin (Baycol), atorvastatin (Lipitor), simvastatin (Zocor), pravastatin (Pravachol), lovastatin (Mevacor) and rosuvastatin (ZD 4522) given orally and as ophthalmic topical preparation with insulin.

19. The method of treating age related macular degeneration according to claim 2 wherein said therapeutic agents are melanin, or melanin-promoting compound; wherein said agent is adapted to be suitable for oral, ophthalmic (including intravitreal or intracorneal or conjunctival sac), nasal, topical, and other parenteral routes.

20. The method of treating age related macular degeneration according to claim 2 wherein said therapeutic agents alters a function a neovascular tissue by using low energy light to generate reactive species within the vessels, or within and around the vessels, to thereby damage these vessels and prevent further growth.

21. The method of treating age related macular degeneration according to claim 2 wherein said therapeutic agents are selected to have antiangiogenesis effects, and anti vascular endothelial growth factors (AVEGF).

22. The method of treating age related macular degeneration according to claim 2, further comprises the step of administering antidiabetic Metformin and related biguanide class of anti-diabetic therapeutic agents and Vitamin K to a patient who has type II diabetics with ARM D.
